(19) United States
(12) Patent Application Publication
(10) Pub. No.: US 2023/0276807 A1
(43) Pub. Date: Sep. 7, 2023

(54) IMPROVED PLANT NITROGEN CONSISTENCY THROUGH THE SUPPLY OF WHOLE PLANT NITROGEN FROM A NITROGEN FIXING MICROBE

(71) Applicant: Pivot Bio, Inc., Berkeley, CA (US)

(72) Inventors: Mark REISINGER, Berkeley, CA (US); Ernest SANDERS, Berkeley, CA (US); Karsten TEMME, Berkeley, CA (US); David BROWN, Berkeley, CA (US); Stephen FICK, Berkeley, CA (US)

(73) Assignee: Pivot Bio, Inc., Berkeley, CA (US)

(21) Appl. No.: 18/020,040

(22) PCT Filed: Aug. 4, 2021
(86) PCT No.: PCT/IB2021/057171

§ 371 Date: Feb. 6, 2023

Related U.S. Application Data
(60) Provisional application No. 63/060,996, filed on Aug. 4, 2020.

(51) Int. Cl. A01N 63/20; G06Q 40/04

(52) U.S. Cl. CPC A01N 63/20; G06Q 40/04

(57) ABSTRACT

Abstract: A method for reducing variation in whole plant nitrogen includes providing, to a locus,a plurality of crop plants and a plurality of nitrogen fixing microbes that colonize the rhizosphere of said plurality of crop plants and supply the plants with fixed N. The variation in whole plant nitrogen of the plurality of crop plants colonized by said nitrogen fixing microbes, at a given growth stage and as measured across the locus, is lower than a variation in whole plant nitrogen of a control plurality of crop plants, when the control plurality of crop plants is provided to the locus.

### CROSS-REFERENCE TO RELATED APPLICATIONS

This application claims the benefit of, and priority to, U.S. Provisional Pat. Application No. 63/060,996, filed Aug. 4, 2020 and titled “Improved Plant Nitrogen Consistency Through the Supply of Whole Plant Nitrogen from a Nitrogen Fixing Microbe,” and this application is related to International Patent Application No. PCT/US2020/016471, filed Feb. 4, 2020 and titled “Improved Consistency of Crop Yield Through Biological Nitrogen Fixation,” the entire contents of each of which are herein incorporated by reference in their entireties.

### STATEMENT REGARDING SEQUENCE LISTING

The contents of the text file submitted electronically herewith are incorporated herein by reference in their entirety: A computer readable format copy of the Sequence Listing filename: PIVO_013_03WO_SeqList_ST25.txt, date created, Jul. 16, 2021, file size ≈ 634 kilobytes.

### BACKGROUND OF THE DISCLOSURE

Nitrogen is the fourth most comment element in plant tissue, used primarily by the plant to construct the enzymes needed for photosynthesis. As the plant grows, it needs to have continuous access to nitrogen in order to build more photosynthetic machinery. Nitrogen deficiency, however, can lead to defects in growth, and if the nitrogen supply is variable in a given agriculture field, there may be a variability in growth and of the plants in the field. A more consistent nitrogen supply to plants in a field is therefore desirable.

Crop yield is an important factor in agriculture, however it is not possible to measure crop yield before the crop of plants is harvested. A method of accurately predicting yield using a highly correlative proxy variable prior to harvesting (i.e., at any point in the plant’s life cycle) is needed.

### SUMMARY OF THE DISCLOSURE

The present disclosure describes methods for reducing variation in whole plant nitrogen and/or increasing the nitrogen consistency of a plant using microbes/compositions that supply crop plants with sustainable biologically fixed N. In some embodiments, a method includes providing to a locus a plurality of crop plants and a plurality of nitrogen fixing microbes that colonize the rhizosphere of said plurality of crop plants and supply the plants with fixed N. The plurality of nitrogen fixing microbes can include at least one of a wild type microbe, an engineered microbe, a transgenic microbe, an intragenic microbe, a remodeled microbe, and a non-intergeneric remodeled microbe. The nitrogen fixing microbes can be provided via in-furrow treatment, or via a seed coat, or via any agricuturaly acceptable methodology of delivery. The locus can comprise agriculturally challenging soil. The variation in whole plant nitrogen of the plurality of crop plants (e.g., cereal crops) colonized by said nitrogen fixing microbes, at a given growth stage and as measured across the locus, is lower than a variation in whole plant nitrogen of a control plurality of crop plants, when the control plurality of crop plants is provided to the locus. The given growth stage can be a vegetative growth stage: in aspects, the vegetative growth stage can be VE to V14, in aspects the vegetative growth stage can be VE to VT, in aspects the vegetative growth stage can be any vegetative growth stage depicted in FIG. 11, in aspects the vegetative growth stage can be of between V1 and V9, inclusive, or at or before about V6, or at or about V7, or at or about VT. The given growth stage can be a reproductive growth stage: in aspects, the reproductive growth stage can be any R stage, in aspects the reproductive growth stage can be any reproductive growth stage depicted in FIG. 11, in aspects the reproductive growth stage can be of between R1 and R6, inclusive, or at or before R3. The variation in whole plant nitrogen can be lower at both about the V6 growth stage and about the R6 growth stage. The variation in whole plant nitrogen of the plurality of crop plants colonized by the nitrogen fixing microbes can be at least about 50%, 40%, 30%, 25% 20%, 15%, 10%, or 5% lower than the variation in whole plant nitrogen of the control plurality of crop plants. In aspects, the “control” plurality of crop plants have been fertilized with a standard synthetic N regime, but have not been treated with a N fixing microbe.

In some embodiments, the nitrogen fixing microbes produce in the aggregate at least about 15 pounds of fixed N per acre over the course of at least about 10 days to about 60 days. Alternatively or in addition, the plurality of nitrogen fixing microbes can comprise at least two different species of bacteria. Alternatively or in addition, the plurality of nitrogen fixing microbes can comprise at least two different strains of the same species of bacteria.

In some embodiments, the nitrogen fixing microbes each produce fixed N of at least about 2.75 × 10⁻¹² mmol of N per CFU per hour. The nitrogen fixing microbes can be capable of fixing atmospheric nitrogen in the presence of exogenous nitrogen. In some embodiments, each member of the plurality of nitrogen fixing microbes comprises at least one genetic variation introduced into at least one gene, or non-coding polynucleotide, of the nitrogen fixation or assimilation genetic regulatory network.

In some embodiments, the variation in the whole plant nitrogen of the plurality of crop plants colonized by the nitrogen fixing microbes, at the given growth stage and as measured across the locus, has a value that is dependent upon an associated nitrogen fertilization rate. In a first example implementation, the nitrogen fertilization rate is at least about 150 pounds of N per acre, and results in a reduction in variance of at least about 200 pounds of N per acre. In a second example implementation, the nitrogen fertilization rate is at least about 175 pounds of N per acre, and results in a reduction in variance of at least about 400 pounds of N per acre.

In some embodiments, a plurality of crop plants having reduced variation in whole plant nitrogen, in an agricultural locus, relative to a control set of crop plants, comprises a plurality of crop plants (e.g., cereal crops) at a given growth stage, in association with a plurality of nitrogen fixing microbes, whereby the plurality of crop plants receive at least 1% of their in planta fixed N from the microbes. The plurality of nitrogen fixing microbes can include at least one of a wild type microbe, an engineered microbe, a transgenic microbe, an intragenic microbe, a remodeled microbe, and a non-intergeneric remodeled microbe. The nitrogen fixing microbes can be associated with the plurality of crop plants in-furrow. The locus can comprise agriculturally challenging soil. The variation in whole plant nitrogen measured across the locus can be lower for the plurality of crop plants in association with said nitrogen fixing microbes, as compared to a control plurality of crop plants, when the control plurality of crop plants at the given growth stage is provided to the locus. The given growth stage can be a vegetative growth stage of between V1 and V9, inclusive, or at or before about V6, or at or about V7, or at or about VT, or a reproductive growth stage of between R1 and R6, inclusive, or at or before R3. The variation in whole plant nitrogen can be lower at both about the V6 growth stage and about the R6 growth stage. The variation in whole plant nitrogen of the plurality of crop plants in association with the nitrogen fixing microbes can be at least about 15% lower than that of the control plurality of crop plants.

In some embodiments, the nitrogen fixing microbes produce in the aggregate at least about 15 pounds of fixed N per acre over the course of at least about 10 days to about 60 days. Alternatively or in addition, the plurality of nitrogen fixing microbes can comprise at least two different species of bacteria. Alternatively or in addition, the plurality of nitrogen fixing microbes can comprise at least two different strains of the same species of bacteria.

In some embodiments, the nitrogen fixing microbes each produce fixed N of at least about 2.75 × 10⁻¹² mmol of N per CFU per hour. The nitrogen fixing microbes can be capable of fixing atmospheric nitrogen in the presence of exogenous nitrogen. In some embodiments, each member of the plurality of nitrogen fixing microbes comprises at least one genetic variation introduced into at least one gene, or non-coding polynucleotide, of the nitrogen fixation or assimilation genetic regulatory network.

### BRIEF DESCRIPTION OF THE DRAWINGS

FIG. 1 depicts the type, energy source, and fixation capabilities of biological N₂ fixation systems in soils.

FIG. 2 depicts the nitrogen needs of a corn plant throughout the growing season. In order for a nitrogen fixing microbe to supply a corn plant with all of its nitrogen needs over a growing season, and thus completely replace synthetic fertilizer, then the microbes (in the aggregate) need to produce about 200 pounds of nitrogen per acre. FIG. 2 also illustrates that strain PBC 137-1036 (i.e. the remodeled Klebsiella variicola) supplies about 20 pounds of nitrogen per acre.

FIG. 3A provides a scenario whereby fertilizer could be replaced by the remodeled microbes of the disclosure. As aforementioned in FIG. 2, the large dashed line is the nitrogen required by the corn (about 200 pounds per acre). The solid line, as already discussed, is the current nitrogen amount that can be supplied by the remodeled 137-1036 strain (about 20 pounds per acre). In the “A” bubble scenario, the inventors expect to increase the activity of the 137-1036 strain by 5 fold (see FIGS. 4A-4B for GMR campaign strategy to achieve such). In the “B” scenario, the inventors expect to utilize a remodeled microbe with a particular colonization profile that is complementary to that of the 137-1036 strain, and which will supply nitrogen to the plant at later stages of the growth cycle.

FIG. 3B shows the nitrogen production by a further remodeled strain 137-3890 at the time of the present application relative to the nitrogen production by the strain 137-1036 from the time of the provisional application. The dashed line indicates the nitrogen needs of a corn plant throughout the growing season.

FIG. 4A illustrates genetic features (i.e. non-intergeneric genetic modifications) that were used with respect to a GMR campaign for PBC6.1 (Kosakonia sacchari). As can be seen, the predicted N produced (lbs of N per acre) increased with each additional feature engineered into the microbial strain. In addition to the GMR campaign for PBC6.1 depicted in FIG. 4A, one can also see the GMR campaign being executed for the PBC137 (Klebsiella variicola). At the time of the provisional application, the nitrogenase expression feature (F1) had been engineered into the host strain. Features 2-6 were being executed and their expected contribution to N produced (lbs of N per acre) at the time the provisional application was filed is depicted by the dashed bar graphs. These expectations were informed by the data from the PBC6.1 GMR campaign. As can be seen in FIG. 3A scenario “A”, once the GMR campaign is completed in PBC137, it is anticipated that the non-intergeneric remodeled strain (in the aggregate, considering all microbes/colonized plants in an acre) will be capable of supplying nearly all of the nitrogen needs of a corn plant throughout the plant’s early growth cycle.

FIG. 4B illustrates genetic features (i.e. non-intergeneric genetic modifications) that were used with respect to a GMR campaign for PBC6.1 (Kosakonia sacchari). As can be seen, the predicted N produced (lbs of N per acre) increased with each additional feature engineered into the microbial strain. In addition to the GMR campaign for PBC6.1 depicted in FIG. 4A, one can also see the GMR campaign being executed for the PBC137 (Klebsiella variicola). Currently, features F1-F3 have been engineered into the host strain and features F4-F6 are being executed. As can be seen in FIG. 3A scenario “A”, once the GMR campaign is completed in PBC137, it is anticipated that the non-intergeneric remodeled strain (in the aggregate, considering all microbes/colonized plants in an acre) will be capable of supplying nearly all of the nitrogen needs of a corn plant throughout the plant’s early growth cycle.

FIG. 5A depicts the same expectation as presented in FIG. 4A, and maps the expected gains in nitrogen production to the applicable feature set.

FIG. 5B depicts N produced as mmol of N/CFU per hour by the remodeled strains of PBC137 once the features F1 (nitrogenase expression), F2 (nitrogen assimilation), and F3 (ammonium excretion) were incorporated.

FIG. 6 depicts the colonization days 1-130 and the total CFU per acre of the non-intergeneric remodeled microbe of 137-1036

FIG. 7 depicts the colonization days 1-130 and the total CFU per acre of the proposed non-intergeneric remodeled microbe (progeny of 137-1036, see FIGS. 4A-4B and FIGS. 5A-5B for proposed genetic alteration features),

FIG. 8 depicts the colonization days 1-130 and the total CFU per acre of a proposed non-intergeneric remodeled microbe that has a complimentary colonization profile to the 137-1036 microbe. As mentioned, this microbe is expected to produce about 100 pounds of nitrogen per acre (in the aggregate) (scenario “B” in FIGS. 3A-3B), and should start colonizing at about the same time that the 137-1036 microbe begins to decline.

FIG. 9 provides the colonization profile of the 137-1036 in the top panel and the colonization profile of the microbe with a later stage/complimentary colonization dynamic in the bottom panel.

FIG. 10 depicts two scenarios: (1) the colonization days 1-130 and the total CFU per acre of a proposed consortia of non-intergeneric remodeled microbes that have a colonization profile as depicted, or (2) the colonization days 1-130 and the total CFU per acre of a proposed single non-intergeneric remodeled microbe that has the depicted colonization profile.

FIG. 11 sets forth the general experimental design utilized in Example 3, which entailed collecting colonization and transcript samples from corn over the course of 10 weeks. These samples allowed for the calculation of colonization ability of the microbes, as well as activity of the microbes.

FIG. 12 provides a visual representation of aspects of the sampling scheme utilized in Example 3, which allows for differentiation of colonization patterns between a “standard” seminal node root sample and a more “peripheral” root sample.

FIG. 13 provides a visual representation of aspects of the sampling scheme utilized in Example 3.

FIG. 14 illustrates that the WT 137 (Klebsiella variicola), 019 (Rahnella aquatilis), and 006 (Kosakonia sacchari), all have a similar colonization pattern.

FIG. 15 depicts the experimental scheme utilized to sample the corn roots in Example 3. The plots: each square is a time point, the Y axis is the distance, and the X axis is the node. The standard sample was always collected along with the leading edge of growth. The periphery and intermediate samples changed week to week, but an attempt at consistency was made.

FIG. 16 depicts the overall results from the Example 3, which utilized and averaged all the data taken in the sampling scheme of FIG. 15. As can be seen from FIG. 16, strain 137 maintains higher colonization in peripheral roots than strain 6 or strain 19. The ‘standard sample’ was most representative for this strain when compared to samples from other root locations.

FIG. 17 depicts NDVI data illustrating that the microbes of the disclosure enable reduced infield variability of a corn crop exposed to said microbes, which translates into improved yield stability for the farmer.

FIG. 18 depicts the amount of ammonium excreted from eight remodeled bacterial strains. Strain 137-1036 is estimated to produce 22.15 pounds of nitrogen per acre. Strain 137-2084 is estimated to produce 38.77 pounds of nitrogen per acre. Strain 137-2219 is estimated to produce 75.74 pounds of nitrogen per acre.

FIG. 19 depicts data collection (299,460 data points analyzed on this farm) and quality control for harvest combine monitor data for an example field treated with 137-1036 or standard agronomic practice. Data were removed where the harvest combine did not have a steady velocity and are illustrated as white gaps on the field plot image.

FIG. 20 illustrates an example distribution plot for yield on a single farm. The standard deviation for farm acreage treated with the remodeled microbe 137-1036 (32.2 yield stdev bu/acre) had lower standard deviation in yield, as compared to a “control” farm (47.3 yield stdev bu/acre) that did not utilize the 137-1036, but rather only utilized the Grower Standard Practice (GSP), i.e. synthetic N application.

FIG. 21 illustrates an example distribution plot for yield on a single farm. The standard deviation for farm acreage treated with the remodeled microbe 137-1036 (34.3 yield stdev bu/acre) had lower standard deviation in yield, as compared to a “control” farm (47.2 yield stdev bu/acre) that did not utilize the 137-1036, but rather only utilized the Grower Standard Practice (GSP), i.e. synthetic N application.

FIG. 22 illustrates an example distribution plot for yield on a single farm. The standard deviation for farm acreage treated with the remodeled microbe 137-1036 (33.7 yield stdev bu/acre) had lower standard deviation in yield, as compared to a “control” farm (42.7 yield stdev bu/acre) that did not utilize the 137-1036, but rather only utilized the Grower Standard Practice (GSP), i.e. synthetic N application.

FIG. 23 illustrates an example distribution plot for yield on a single farm. The standard deviation for farm acreage treated with the remodeled microbe 137-1036 (17.4 yield stdev bu/acre) had lower standard deviation in yield, as compared to a “control” farm (26.0 yield stdev bu/acre) that did not utilize the 137-1036, but rather only utilized the Grower Standard Practice (GSP), i.e. synthetic N application.

FIG. 24 illustrates yield consistency improvement and variance reduction between 137-1036 treated and untreated (Grower Standard Practice) control by farm. 64% of farms showed an improvement with a smaller standard deviation ranging from 0.8 to 15.1 bu/acre. Blue bars indicate a significant difference, grey bars (asterick) indicate the difference was not significant

FIG. 25 is a system diagram for the transacting of financial and insurance instruments, according to some embodiments.

FIG. 26 is a flow diagram illustrating a method for determining a quantity of a crop plant to sell based on whole plant nitrogen variability data for a bacteria-colonized plant, according to some embodiments.

FIG. 27 is a flow diagram illustrating a method for pricing and transacting an insurance product insurance policy, based on whole plant nitrogen variability data for a bacteria-colonized plant, according to some embodiments.

### DETAILED DESCRIPTION OF THE DISCLOSURE

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

Nitrogen is a vital nutrient for plants, for example because it is a major component of the chlorophyll molecule, which produces food for the plants through photosynthesis, and because it is a primary building block for plant protoplasm. Nitrogen deficiency can lead to a variety of issues, including defects in growth. If the nitrogen supply is inconsistent across a given agriculture field in which the plants are grown, the growth of the plants in that agriculture field (and, thus, the yield associated with those plants) may be highly variable and/or unpredictable. Unfortunately, it is not possible to measure crop yield before the crop of plants is harvested. A method of accurately predicting yield using a highly correlative proxy variable prior to harvesting (i.e., at any point in the plant’s life cycle) is needed.

The present disclosure describes methods that overcome the aforementioned problems, advantageously reducing variation in whole plant nitrogen and/or increasing the nitrogen consistency of a plant, using microbes/compositions that supply crop plants with sustainable biologically fixed N. In some embodiments, a method includes providing to a locus a plurality of crop plants and a plurality of nitrogen fixing microbes that colonize the rhizosphere of said plurality of crop plants and supply the plants with fixed N. The whole plant nitrogen of the plurality of crop plants can be measured at any growth stage during the life cycle of the crop plants.

### Definitions

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner according to base complementarity. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the enzymatic cleavage of a polynucleotide by an endonuclease. A second sequence that is complementary to a first sequence is referred to as the “complement” of the first sequence. The term “hybridizable” as applied to a polynucleotide refers to the ability of the polynucleotide to form a complex that is stabilized via hydrogen bonding between the bases of the nucleotide residues in a hybridization reaction.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. Sequence identity, such as for the purpose of assessing percent complementarity, may be measured by any suitable alignment algorithm, including but not limited to the Needleman-Wunsch algorithm (see e.g. the EMBOSS Needle aligner available at www.ebi.ac.uk/Tools/psa/emboss_needle/nucleotide.html, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebt.ac.uk/Tools/psa/emboss_water/nucleotide.html, optionally with default settings). Optimal alignment may be assessed using any suitable parameters of a chosen algorithm, including default parameters.

In general, “stringent conditions” for hybridization refer to conditions under which a nucleic acid having complementarity to a target sequence predominantly hybridizes with a target sequence, and substantially does not hybridize to non-target sequences. Stringent conditions are generally sequence-dependent and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, the term “about” is used synonymously with the term “approximately.” Illustratively, the use of the term “about” with regard to an amount indicates that values slightly outside the cited values, e.g., plus or minus 0.1% to 10%.

The term “biologically pure culture” or “substantially pure culture” refers to a culture of a bacterial species described herein containing no other bacterial species in quantities sufficient to interfere with the replication of the culture or be detected by normal bacteriological techniques.

“Plant productivity” refers generally to any aspect of growth or development of a plant that is a reason for which the plant is grown. For food crops, such as grains or vegetables, “plant productivity” can refer to the yield of grain or fruit harvested from a particular crop. As used herein, improved plant productivity refers broadly to improvements in yield of grain, fruit, flowers, or other plant parts harvested for various purposes, improvements in growth of plant parts, including stems, leaves and roots, promotion of plant growth, maintenance of high chlorophyll content in leaves, increasing fruit or seed numbers, increasing fruit or seed unit weight, reducing NO₂ emission due to reduced nitrogen fertilizer usage and similar improvements of the growth and development of plants.

Microbes in and around food crops can influence the traits of those crops. Plant traits that may be influenced by microbes include: yield (e.g., grain production, biomass generation, fruit development, flower set); nutrition (e.g., nitrogen, phosphorus, potassium, iron, micronutrient acquisition); abiotic stress management (e.g., drought tolerance, salt tolerance, heat tolerance); and biotic stress management (e.g., pest, weeds, insects, fungi, and bacteria). Strategies for altering crop traits include: increasing key metabolite concentrations; changing temporal dynamics of microbe influence on key metabolites; linking microbial metabolite production/degradation to new environmental cues; reducing negative metabolites; and improving the balance of metabolites or underlying proteins.

As used herein, a “control sequence” refers to an operator, promoter, silencer, or terminator.

As used herein, “in planta” may refer to in the plant, on the plant, or intimately associated with the plant, depending upon context of usage (e.g. endophytic, epiphytic, or rhizospheric associations). The plant may comprise plant parts, tissue, leaves, roots, root hairs, rhizomes, stems, seed, ovules, pollen, flowers, fruit, etc.

In some embodiments, native or endogenous control sequences of genes of the present disclosure are replaced with one or more intrageneric control sequences.

As used herein, “introduced” refers to the introduction by means of modern biotechnology, and not a naturally occurring introduction.

In some embodiments, the bacteria of the present disclosure have been modified such that they are not naturally occurring bacteria.

In some embodiments, the bacteria of the present disclosure are present in the plant in an amount of at least 10³ cfu, 10⁴ cfu, 10⁵ cfu, 10⁶ cfu, 10⁷ cfu, 10⁸ cfu, 10⁹ cfu, 10¹⁰ cfu, 10¹¹ cfu, or 10¹² cfu per gram of fresh or dry weight of the plant. In some embodiments, the bacteria of the present disclosure are present in the plant in an amount of at least about 10³ cfu, about 10⁴ cfu, about 10⁵ cfu, about 10⁶ cfu, about 10⁷ cfu, about 10⁸ cfu, about 10⁹ cfu, about 10¹⁰ cfu, about 10¹¹ cfu, or about 10¹² cfu per gram of fresh or dry weight of the plant. In some embodiments, the bacteria of the present disclosure are present in the plant in an amount of at least 10³ to 10⁹, 10³ to 10⁷, 10³ to 10⁵, 10⁵ to 10⁹, 10⁵ to 10⁷, 10⁶ to 10¹⁰, 10⁶ to 10⁷ cfu per gram of fresh or dry weight of the plant.

Fertilizers and exogenous nitrogen of the present disclosure may comprise the following nitrogen-containing molecules: ammonium, nitrate, nitrite, ammonia, glutamine, etc. Nitrogen sources of the present disclosure may include anhydrous ammonia, ammonia sulfate, urea, diammonium phosphate, urea-form, monoammonium phosphate, ammonium nitrate, nitrogen solutions, calcium nitrate, potassium nitrate, sodium nitrate, etc.

As used herein, “exogenous nitrogen” refers to non-atmospheric nitrogen readily available in the soil, field, or growth medium that is present under non-nitrogen limiting conditions, including ammonia, ammonium, nitrate, nitrite, urea, uric acid, ammonium acids, etc.

As used herein, “non-nitrogen limiting conditions” refers to non-atmospheric nitrogen available in the soil, field, media at concentrations greater than about 4 mM nitrogen, as disclosed by Kant et al. (2010. J. Exp. Biol. 62(4):1499-1509), which is incorporated herein by reference.

As used herein, an “intergeneric microorganism” is a microorganism that is formed by the deliberate combination of genetic material originally isolated from organisms of different taxonomic genera. An “intergeneric mutant” can be used interchangeably with “intergeneric microorganism”. An exemplary “intergeneric microorganism” includes a microorganism containing a mobile genetic element which was first identified in a microorganism in a genus different from the recipient microorganism. Further explanation can be found, inter alia, in 40 C.F.R. § 725.3.

In aspects, microbes taught herein are “non-intergeneric,” which means that the microbes are not intergeneric.

As used herein, an “intrageneric microorganism” is a microorganism that is formed by the deliberate combination of genetic material originally isolated from organisms of the same taxonomic genera. An “intrageneric mutant” can be used interchangeably with “intrageneric microorganism.”

As used herein, “introduced genetic material” means genetic material that is added to, and remains as a component of, the genome of the recipient.

As used herein, in the context of non-intergeneric microorganisms, the term “remodeled” is used synonymously with the term “engineered”. Consequently, a “non-intergeneric remodeled microorganism” has a synonymous meaning to “non-intergeneric engineered microorganism,” and will be utilized interchangeably. Further, the disclosure may refer to an “engineered strain” or “engineered derivative” or “engineered non-intergeneric microbe,” these terms are used synonymously with “remodeled strain” or “remodeled derivative” or “remodeled non-intergeneric microbe.”

In some embodiments, the nitrogen fixation and assimilation genetic regulatory network comprises polynucleotides encoding genes and non-coding sequences that direct, modulate, and/or regulate microbial nitrogen fixation and/or assimilation and can comprise polynucleotide sequences of the nif cluster (e.g., nifA, nifB, nifC,.......nifZ), polynucleotides encoding nitrogen regulatory protein C, polynucleotides encoding nitrogen regulatory protein B, polynucleotide sequences of the gln cluster (e.g. glnA and glnD), draT, and ammonia transporters/permeases. In some cases, the Nif cluster may comprise NifB, NifH, NifD, NifK, NifE, NifN, NifX, hesa, and NifV. In some cases, the Nif cluster may comprise a subset of NifB, NifH, NifD, NifK, NifE, NifN, NifX, hesa, and NifV.

In some embodiments, fertilizer of the present disclosure comprises at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, %, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% nitrogen by weight.

In some embodiments, fertilizer of the present disclosure comprises at least about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% nitrogen by weight.

In some embodiments, fertilizer of the present disclosure comprises about 5% to 50%, about 5% to 75%, about 10% to 50%, about 10% to 75%, about 15% to 50%, about 15% to 75%, about 20% to 50%, about 20% to 75%, about 25% to 50%, about 25% to 75%, about 30% to 50%, about 30% to 75%, about 35% to 50%, about 35% to 75%, about 40% to 50%, about 40% to 75%, about 45% to 50%, about 45% to 75%, or about 50% to 75% nitrogen by weight.

In some embodiments, the increase of nitrogen fixation and/or the production of 1% or more of the nitrogen in the plant are measured relative to control plants, which have not been exposed to the bacteria of the present disclosure. All increases or decreases in bacteria are measured relative to control bacteria. All increases or decreases in plants are measured relative to control plants. In some embodiments, control plants can include fertilizer-treated plants.

As used herein, a “constitutive promoter” is a promoter, which is active under most conditions and/or during most development stages. There are several advantages to using constitutive promoters in expression vectors used in biotechnology, such as: high level of production of proteins used to select transgenic cells or organisms; high level of expression of reporter proteins or scorable markers, allowing easy detection and quantification; high level of production of a transcription factor that is part of a regulatory transcription system; production of compounds that requires ubiquitous activity in the organism; and production of compounds that are required during all stages of development. Non-limiting exemplary constitutive promoters include, CaMV 35S promoter, opine promoters, ubiquitin promoter, alcohol dehydrogenase promoter, etc.

As used herein, a “non-constitutive promoter” is a promoter which is active under certain conditions, in certain types of cells, and/or during certain development stages. For example, tissue specific, tissue preferred, cell type specific, cell type preferred, inducible promoters, and promoters under development control are non-constitutive promoters. Examples of promoters under developmental control include promoters that preferentially initiate transcription in certain tissues.

As used herein, “inducible” or “repressible” promoter is a promoter which is under chemical or environmental factors control. Examples of environmental conditions that may affect transcription by inducible promoters include anaerobic conditions, certain chemicals, the presence of light, acidic or basic conditions, etc.

As used herein, a “tissue specific” promoter is a promoter that initiates transcription only in certain tissues. Unlike constitutive expression of genes, tissue-specific expression is the result of several interacting levels of gene regulation. As such, in the art sometimes it is preferable to use promoters from homologous or closely related species to achieve efficient and reliable expression of transgenes in particular tissues. This is one of the main reasons for the large amount of tissue-specific promoters isolated from particular tissues found in both scientific and patent literature.

As used herein, the term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the disclosure can be operably linked, either directly or indirectly, 5′ to the target mRNA, or 3′ to the target mRNA, or within the target mRNA, or a first complementary region is 5′ and its complement is 3′ to the target mRNA.

In aspects, “applying to the plant a plurality of non-intergeneric bacteria,” includes any means by which the plant (including plant parts such as a seed, root, stem, tissue, etc.) is made to come into contact (i.e. exposed) with said bacteria at any stage of the plant’s life cycle. Consequently, “applying to the plant a plurality of non-intergeneric bacteria,” includes any of the following means of exposing the plant (including plant parts such as a seed, root, stem, tissue, etc.) to said bacteria: spraying onto plant, dripping onto plant, applying as a seed coat, applying to a field that will then be planted with seed, applying to a field already planted with seed, applying to a field with adult plants, etc.

As used herein “MRTN” is an acronym for maximum return to nitrogen and is utilized as an experimental treatment in the Examples. MRTN was developed by Iowa State University and information can be found at: enre.agron.iastate.edu/. The MRTN is the nitrogen rate where the economic net return to nitrogen application is maximized. The approach to calculating the MRTN is a regional approach for developing corn nitrogen rate guidelines in individual states. The nitrogen rate trial data was evaluated for Illinois, Iowa, Michigan, Minnesota, Ohio, and Wisconsin where an adequate number of research trials were available for corn plantings following soybean and corn plantings following corn. The trials were conducted with spring, sidedress, or split preplant/sidedress applied nitrogen, and sites were not irrigated except for those that were indicated for irrigated sands in Wisconsin. MRTN was developed by Iowa State University due to apparent differences in methods for determining suggested nitrogen rates required for corn production, misperceptions pertaining to nitrogen rate guidelines, and concerns about application rates. By calculating the MRTN, practitioners can determine the following: (1) the nitrogen rate where the economic net return to nitrogen application is maximized, (2) the economic optimum nitrogen rate, which is the point where the last increment of nitrogen returns a yield increase large enough to pay for the additional nitrogen, (3) the value of corn grain increase attributed to nitrogen application, and the maximum yield, which is the yield where application of more nitrogen does not result in a corn yield increase. Thus the MRTN calculations provide practitioners with the means to maximize corn crops in different regions while maximizing financial gains from nitrogen applications.

The term mmol is an abbreviation for millimole, which is a thousandth (10⁻³) of a mole, abbreviated herein as mol.

As used herein the term “plant” can include plant parts, tissue, leaves, roots, root hairs, rhizomes, stems, seeds, ovules, pollen, flowers, fruit, etc. Thus, when the disclosure discusses providing a plurality of corn plants to a particular locus, it is understood that this may entail planting a corn seed at a particular locus.

As used herein the terms “microorgani sm” or “mi crobe” should be taken broadly. These terms, used interchangeably, include but are not limited to, the two prokaryotic domains, Bacteria and Archaea. The term may also encompass eukaryotic fungi and protists.

As used herein, when the disclosure discuses a particular microbial deposit by accession number, it is understood that the disclosure also contemplates a microbial strain having all of the identifying characteristics of said deposited microbe, and/or a mutant thereof.

The term “microbial consortia” or “microbial consortium” refers to a subset of a microbial community of individual microbial species, or strains of a species, which can be described as carrying out a common function, or can be described as participating in, or leading to, or correlating with, a recognizable parameter, such as a phenotypic trait of interest.

The term “microbial community” means a group of microbes comprising two or more species or strains. Unlike microbial consortia, a microbial community does not have to be carrying out a common function, or does not have to be participating in, or leading to, or correlating with, a recognizable parameter, such as a phenotypic trait of interest.

As used herein, “isolate,” “isolated,” “isolated microbe,” and like terms, are intended to mean that the one or more microorganisms has been separated from at least one of the materials with which it is associated in a particular environment (for example soil, water, plant tissue, etc.). Thus, an “isolated microbe” does not exist in its naturally occurring environment; rather, it is through the various techniques described herein that the microbe has been removed from its natural setting and placed into a non-naturally occurring state of existence. Thus, the isolated strain or isolated microbe may exist as, for example, a biologically pure culture, or as spores (or other forms of the strain). In aspects, the isolated microbe may be in association with an acceptable carrier, which may be an agriculturally acceptable carrier.

In certain aspects of the disclosure, the isolated microbes exist as “isolated and biologically pure cultures.” It will be appreciated by one of skill in the art, that an isolated and biologically pure culture of a particular microbe, denotes that said culture is substantially free of other living organisms and contains only the individual microbe in question. The culture can contain varying concentrations of said microbe. The present disclosure notes that isolated and biologically pure microbes often “necessarily differ from less pure or impure materials.” See, e.g. In re Bergstrom, 427 F.2d 1394, (CCPA 1970)(discussing purified prostaglandins), see also, In re Bergy, 596 F.2d 952 (CCPA 1979)(discussing purified microbes), see also, Parke-Davis & Co. v. H.K. Mulford & Co., 189 F. 95 (S.D.N.Y. 1911) (Learned Hand discussing purified adrenaline), aff’d in part, rev’d in part, 196 F. 496 (2d Cir. 1912), each of which are incorporated herein by reference. Furthermore, in some aspects, the disclosure provides for certain quantitative measures of the concentration, or purity limitations, that must be found within an isolated and biologically pure microbial culture. The presence of these purity values, in certain embodiments, is a further attribute that distinguishes the presently disclosed microbes from those microbes existing in a natural state. See, e.g., Merck & Co. v. Olin Mathieson Chemical Corp., 253 F.2d 156 (4th Cir. 1958) (discussing purity limitations for vitamin B12 produced by microbes), incorporated herein by reference.

As used herein, “individual isolates” should be taken to mean a composition, or culture, comprising a predominance of a single genera, species, or strain, of microorganism, following separation from one or more other microorganisms.

Microbes of the present disclosure may include spores and/or vegetative cells. In some embodiments, microbes of the present disclosure include microbes in a viable but non-culturable (VBNC) state. As used herein, “spore” or “spores” refer to structures produced by bacteria and fungi that are adapted for survival and dispersal. Spores are generally characterized as dormant structures; however, spores are capable of differentiation through the process of germination. Germination is the differentiation of spores into vegetative cells that are capable of metabolic activity, growth, and reproduction. The germination of a single spore results in a single fungal or bacterial vegetative cell. Fungal spores are units of asexual reproduction, and in some cases are necessary structures in fungal life cycles. Bacterial spores are structures for surviving conditions that may ordinarily be nonconducive to the survival or growth of vegetative cells.

As used herein, “microbial composition” refers to a composition comprising one or more microbes of the present disclosure. In some embodiments, a microbial composition is administered to plants (including various plant parts) and/or in agricultural fields.

As used herein, “carrier,” “acceptable carrier,” or “agriculturally acceptable carrier” refers to a diluent, adjuvant, excipient, or vehicle with which the microbe can be administered, which does not detrimentally effect the microbe.

As used herein, “whole plant nitrogen” refers to the total amount of accumulated nitrogen in all plant parts, including leave(s), stalk(s), and reproductive tissue(s).

In some embodiments, the microbes and/or genetic modifications disclosed herein are not the microbes taught in PCT/US2018/013671 (WO 2018/132774 A1), filed Jan. 12, 2018, and entitled: Methods and Compositions for Improving Plant Traits. In some embodiments, the methods disclosed herein are not the methods taught in PCT/US2018/013671 (WO 2018/132774 A1), filed Jan. 12, 2018, and entitled: Methods and Compositions for Improving Plant Traits. Thus, the present disclosure contemplates embodiments, which have a negative proviso of the microbes, methods, and gene modifications disclosed in said application.

Details on the regulation of nitrogen fixation, regulation of colonization potential, generation of bacterial populations, domestication of microbes, guided microbial remodeling, etc. can be found in International Patent Application Publication No. WO2020/006246, published January 2^{nd}, 2020 and titled “Guided Microbial Remodeling, A Platform For The Rational Improvement of Microbial Species for Agriculture,” the entire contents of which is incorporated by reference herein for all purposes.

### Improved Plant Nitrogen Consistency

As discussed above, as a plant grows, the plant needs to have continuous access to nitrogen in order to build more photosynthetic machinery. Nitrogen deficiency can therefore lead to a defect in growth. If the nitrogen supply is variable in a given agriculture field, there may be a variability in growth of the plants in the field. A more consistent nitrogen supply to plants in a field, on the other hand, can lead to a decrease in the variance of nitrogen in plants in the field, which in turn can result in higher overall yield, larger plant biomass, and lower overall yield variance. As described in International Patent Application No. PCT/US2020/016471, filed February 4^{th}, 2020 and titled “Improved Consistency of Crop Yield Through Biological Nitrogen Fixation,” such effects are of scientific and commercial interest. Nitrogen variability (e.g., whole plant nitrogen variability), in particular, is also of interest for at least three reasons.

First, while it is not possible to measure yield before a crop of plants is harvested, plant nitrogen can be measured at any point in the plant’s life cycle. A difference in variability in whole plant nitrogen can be predictive of a difference in future yield.

Second, when plant nitrogen is not predictive of yield or yield variance, this may be because factors other than consistency and level of plant nitrogen supply are affecting yield. Such factors may include biological pressures such as weed plant and pest insects, lack of other important plant nutrients such as phosphorus and potassium, and environmental factors that influence plant physiology such as drought and soil water retention. Therefore, variability and level of whole plant nitrogen may be a more specific measure of a nitrogen supply agronomy product than variability and level of yield.

Third, when plant nitrogen is not predictive of yield or yield variance, this may be because the nitrogen supplied to the crop is ample and therefore its variation does not affect yield. Knowledge of this condition would allow an agricultural manager to alter their nitrogen management practices by reducing fertilizer, which would have environmental and economical benefits.

Microbes described herein can fix nitrogen from air, making that nitrogen available to the plants for incorporation. This nitrogen is expected to provide a more consistent supply of nitrogen to the plant than chemical fertilizers, because it is produced in the root zone from which plants uptake nitrogen.

In some embodiments, a method for reducing variation in whole plant nitrogen and/or increasing the nitrogen consistency of a plant includes providing to a locus a plurality of crop plants and a plurality of nitrogen fixing microbes that colonize the rhizosphere of said plurality of crop plants and supply the plants with fixed N. The plurality of nitrogen fixing microbes can include at least one of a wild type microbe, an engineered microbe, a transgenic microbe, an intragenic microbe, a remodeled microbe, and a non-intergeneric remodeled microbe. The nitrogen fixing microbes can be provided via in-furrow treatment. The locus can comprise agriculturally challenging soil. The variation in whole plant nitrogen of the plurality of crop plants (e.g., cereal crops) colonized by said nitrogen fixing microbes, at a given growth stage and as measured across the locus, is lower than a variation in whole plant nitrogen of a control plurality of crop plants, when the control plurality of crop plants is provided to the locus. The given growth stage can be a vegetative growth stage of between V1 and V9, inclusive, or at or before about V6, or at or about V7, or at or about VT, or a reproductive growth stage of between R1 and R6, inclusive, or at or before R3. The variation in whole plant nitrogen can be lower at both about the V6 growth stage and about the R6 growth stage. The variation in whole plant nitrogen of the plurality of crop plants colonized by the nitrogen fixing microbes can be at least about 15% lower than the variation in whole plant nitrogen of the control plurality of crop plants.

Whole plant nitrogen of a plant can be evaluated at any plant growth stage (including any of the growth stages mentioned above), for example by isolating the aboveground biomass, dividing the plant into partitions (e.g., leaves, stalks, and reproductive tissue), drying, weighing, and grinding the partitions, and analytically determining (e.g., via a combustion technique) the nitrogen content within each partition as well as the total nitrogen accumulation of the plant (optionally in combination with analytical determinations of other plant nutrients). These nitrogen measurements can be compared to similar nitrogen measurements taken of a control plant (e.g., at the same/similar growth stage and/or grown under identical or similar conditions, but without the use of nitrogen fixing microbes) to determine an amount of reduction in variation in whole plant nitrogen of the plant (as compared with the control plant) and/or to determine an amount of increase in the whole plant nitrogen consistency of the plant (as compared with the control plant). As defined herein, a “control plant” can refer to (but is not limited to) a fertilizer-treated plant. Experimental details showing increased nitrogen consistency using microbes of the present disclosure are provided in Examples 9 and 10, below.

### Regulation of Nitrogen Fixation

In some cases, nitrogen fixation pathway may act as a target for genetic engineering and optimization. One trait that may be targeted for regulation by the methods described herein is nitrogen fixation. Nitrogen fertilizer is the largest operational expense on a farm and the biggest driver of higher yields in row crops like corn and wheat. Described herein are microbial products that can deliver renewable forms of nitrogen in non-leguminous crops. While some endophytes have the genetics necessary for fixing nitrogen in pure culture, the fundamental technical challenge is that wild-type endophytes of cereals and grasses stop fixing nitrogen in fertilized fields. The application of chemical fertilizers and residual nitrogen levels in field soils signal the microbe to shut down the biochemical pathway for nitrogen fixation.

Changes to the transcriptional and post-translational levels of components of the nitrogen fixation regulatory network may be beneficial to the development of a microbe capable of fixing and transferring nitrogen to corn in the presence of fertilizer. To that end, described herein is Host-Microbe Evolution (HoME) technology to precisely evolve regulatory networks and elicit novel phenotypes. Also described herein are unique, proprietary libraries of nitrogen-fixing endophytes isolated from corn, paired with extensive omics data surrounding the interaction of microbes and host plant under different environmental conditions like nitrogen stress and excess. In some embodiments, this technology enables precision evolution of the genetic regulatory network of endophytes to produce microbes that actively fix nitrogen even in the presence of fertilizer in the field. Also described herein are evaluations of the technical potential of evolving microbes that colonize corn root tissues and produce nitrogen for fertilized plants and evaluations of the compatibility of endophytes with standard formulation practices and diverse soils to determine feasibility of integrating the microbes into modern nitrogen management strategies.

In order to utilize elemental nitrogen (N) for chemical synthesis, life forms combine nitrogen gas (N₂) available in the atmosphere with hydrogen in a process known as nitrogen fixation. Because of the energy-intensive nature of biological nitrogen fixation, diazotrophs (bacteria and archaea that fix atmospheric nitrogen gas) have evolved sophisticated and tight regulation of the nif gene cluster in response to environmental oxygen and available nitrogen. Nif genes encode enzymes involved in nitrogen fixation (such as the nitrogenase complex) and proteins that regulate nitrogen fixation. Shamseldin (2013. Global J. Biotechnol. Biochem. 8(4):84-94) discloses detailed descriptions of nif genes and their products, and is incorporated herein by reference. Described herein are methods of producing a plant with an improved trait comprising isolating bacteria from a first plant, introducing a genetic variation into a gene of the isolated bacteria to increase nitrogen fixation, exposing a second plant to the variant bacteria, isolating bacteria from the second plant having an improved trait relative to the first plant, and repeating the steps with bacteria isolated from the second plant.

In Proteobacteria, regulation of nitrogen fixation centers around the σ₅₄-dependent enhancer-binding protein NifA, the positive transcriptional regulator of the nif cluster. Intracellular levels of active NifA are controlled by two key factors: transcription of the nifLA operon, and inhibition of NifA activity by protein-protein interaction with NifL. Both of these processes are responsive to intracellular glutamine levels via the PII protein signaling cascade. This cascade is mediated by GlnD, which directly senses glutamine and catalyzes the uridylylation or deuridylylation of two PII regulatory proteins - GlnB and GlnK - in response the absence or presence, respectively, of bound glutamine. Under conditions of nitrogen excess, unmodified GlnB signals the deactivation of the nifLA promoter. However, under conditions of nitrogen limitation, GlnB is post-translationally modified, which inhibits its activity and leads to transcription of the nifLA operon. In this way, nifLA transcription is tightly controlled in response to environmental nitrogen via the PII protein signaling cascade. On the post-translational level of NifA regulation, GlnK inhibits the NifL/NifA interaction in a matter dependent on the overall level of free GlnK within the cell.

NifA is transcribed from the nifLA operon, whose promoter is activated by phosphorylated NtrC, another σ₅₄-dependent regulator. The phosphorylation state of NtrC is mediated by the histidine kinase NtrB, which interacts with deuridylylated GlnB but not uridylylated GlnB. Under conditions of nitrogen excess, a high intracellular level of glutamine leads to deuridylylation of GlnB, which then interacts with NtrB to deactivate its phosphorylation activity and activate its phosphatase activity, resulting in dephosphorylation of NtrC and the deactivation of the nifLA promoter. However, under conditions of nitrogen limitation, a low level of intracellular glutamine results in uridylylation of GlnB, which inhibits its interaction with NtrB and allows the phosphorylation of NtrC and transcription of the nifLA operon. In this way, nifLA expression is tightly controlled in response to environmental nitrogen via the PII protein signaling cascade. nifA, ntrB, ntrC, and glnB, are all genes that can be mutated in the methods described herein. These processes may also be responsive to intracellular or extracellular levels of ammonia, urea or nitrates.

The activity of NifA is also regulated post-translationally in response to environmental nitrogen, most typically through NifL-mediated inhibition of NifA activity. In general, the interaction of NifL and NifA is influenced by the PII protein signaling cascade via GlnK, although the nature of the interactions between GlnK and NifL/NifA varies significantly between diazotrophs. In Klebsiella pneumoniae, both forms of GlnK inhibit the NifL/NifA interaction, and the interaction between GlnK and NifL/NifA is determined by the overall level of free GlnK within the cell. Under nitrogen-excess conditions, deuridylylated GlnK interacts with the ammonium transporter AmtB, which serves to both block ammonium uptake by AmtB and sequester GlnK to the membrane, allowing inhibition of NifA by NifL. On the other hand, in Azotobacter vinelandii, interaction with deuridylylated GlnK is required for the NifL/NifA interaction and NifA inhibition, while uridylylation of GlnK inhibits its interaction with NifL. In diazotrophs lacking the nifL gene, there is evidence that NifA activity is inhibited directly by interaction with the deuridylylated forms of both GlnK and GlnB under nitrogen-excess conditions. In some bacteria the Nif cluster may be regulated by glnR, and further in some cases this may comprise negative regulation. Regardless of the mechanism, post-translational inhibition of NifA is an important regulator of the nif cluster in most known diazotrophs. Additionally, nifL, amtB, glnK, and glnR are genes that can be mutated in the methods described herein.

In addition to regulating the transcription of the nif gene cluster, many diazotrophs have evolved a mechanism for the direct post-translational modification and inhibition of the nitrogenase enzyme itself, known as nitrogenase shutoff. This is mediated by ADP-ribosylation of the Fe protein (NifH) under nitrogen-excess conditions, which disrupts its interaction with the MoFe protein complex (NifDK) and abolishes nitrogenase activity. DraT catalyzes the ADP-ribosylation of the Fe protein and shutoff of nitrogenase, while DraG catalyzes the removal of ADP-ribose and reactivation of nitrogenase. As with nifLA transcription and NifA inhibition, nitrogenase shutoff is also regulated via the PII protein signaling cascade. Under nitrogen-excess conditions, deuridylylated GlnB interacts with and activates DraT, while deuridylylated GlnK interacts with both DraG and AmtB to form a complex, sequestering DraG to the membrane. Under nitrogen-limiting conditions, the uridylylated forms of GlnB and GlnK do not interact with DraT and DraG, respectively, leading to the inactivation of DraT and the diffusion of DraG to the Fe protein, where it removes the ADP-ribose and activates nitrogenase. The methods described herein also contemplate introducing genetic variation into the nifH, nifD, nifK, and draT genes.

Although some endophytes have the ability to fix nitrogen in vitro, often the genetics are silenced in the field by high levels of exogenous chemical fertilizers. One can decouple the sensing of exogenous nitrogen from expression of the nitrogenase enzyme to facilitate field-based nitrogen fixation. Improving the integral of nitrogenase activity across time further serves to augment the production of nitrogen for utilization by the crop. Specific targets for genetic variation to facilitate field-based nitrogen fixation using the methods described herein include one or more genes selected from the group consisting of nif4, nifL, ntrB, ntrC, glnA, glnB, glnK, draT, amtB, glnD, glnE, nifJ, nifH, nifD, nifK, nifY, nifE, nifN, nifU, nifS, nifV, nifW, nifZ, nifM, nifF, nifB, and nifQ.

An additional target for genetic variation to facilitate field-based nitrogen fixation using the methods described herein is the NifA protein. The NifA protein is typically the activator for expression of nitrogen fixation genes. Increasing the production of NifA (either constitutively or during high ammonia condition) circumvents the native ammonia-sensing pathway. In addition, reducing the production of NifL proteins, a known inhibitor of NifA, also leads to an increased level of freely active NifA. In addition, increasing the transcription level of the nifAL operon (either constitutively or during high ammonia condition) also leads to an overall higher level of NifA proteins. Elevated level of nifAL expression is achieved by altering the promoter itself or by reducing the expression of NtrB (part of ntrB and ntrC signaling cascade that originally would result in the shutoff of nifAL operon during high nitrogen condition). High level of NifA achieved by these or any other methods described herein increases the nitrogen fixation activity of the endophytes.

Another target for genetic variation to facilitate field-based nitrogen fixation using the methods described herein is the GlnD/GlnB/GlnK PII signaling cascade. The intracellular glutamine level is sensed through the GlnD/GlnB/GlnK PII signaling cascade. Active site mutations in GlnD that abolish the uridylyl-removing activity of GlnD disrupt the nitrogen-sensing cascade. In addition, reduction of the GlnB concentration short circuits the glutamine-sensing cascade. These mutations “trick” the cells into perceiving a nitrogen-limited state, thereby increasing the nitrogen fixation level activity. These processes may also be responsive to intracellular or extracellular levels of ammonia, urea or nitrates.

The amtB protein is also a target for genetic variation to facilitate field-based nitrogen fixation using the methods described herein. Ammonia uptake from the environment can be reduced by decreasing the expression level of amtB protein. Without intracellular ammonia, the endophyte is not able to sense the high level of ammonia, preventing the down-regulation of nitrogen fixation genes. Any ammonia that manages to get into the intracellular compartment is converted into glutamine. Intracellular glutamine level is the major currency of nitrogen sensing. Decreasing the intracellular glutamine level prevents the cells from sensing high ammonium levels in the environment. This effect can be achieved by increasing the expression level of glutaminase, an enzyme that converts glutamine into glutamate. In addition, intracellular glutamine can also be reduced by decreasing glutamine synthase (an enzyme that converts ammonia into glutamine). In diazotrophs, fixed ammonia is quickly assimilated into glutamine and glutamate to be used for cellular processes. Disruptions to ammonia assimilation may enable diversion of fixed nitrogen to be exported from the cell as ammonia. The fixed ammonia is predominantly assimilated into glutamine by glutamine synthetase (GS), encoded by glnA, and subsequently into glutamine by glutamine oxoglutarate aminotransferase (GOGAT). In some examples, glnS encodes a glutamine synthetase. GS is regulated post-translationally by GS adenylyl transferase (GlnE), a bi-functional enzyme encoded by glnE that catalyzes both the adenylylation and de-adenylylation of GS through activity of its adenylyl-transferase (AT) and adenylyl-removing (AR) domains, respectively. Under nitrogen limiting conditions, glnA is expressed, and GlnE’s AR domain de-adynylylates GS, allowing it to be active. Under conditions of nitrogen excess, glnA expression is turned off, and GlnE’s AT domain is activated allosterically by glutamine, causing the adenylylation and deactivation of GS.

Furthermore, the draT gene may also be a target for genetic variation to facilitate field-based nitrogen fixation using the methods described herein. Once nitrogen fixing enzymes are produced by the cell, nitrogenase shut-off represents another level in which cell downregulates fixation activity in high nitrogen condition. This shut-off could be removed by decreasing the expression level of DraT.

Methods for imparting new microbial phenotypes can be performed at the transcriptional, translational, and post-translational levels. The transcriptional level includes changes at the promoter (such as changing sigma factor affinity or binding sites for transcription factors, including deletion of all or a portion of the promoter) or changing transcription terminators and attenuators. The translational level includes changes at the ribosome binding sites and changing mRNA degradation signals. The post-translational level includes mutating an enzyme’s active site and changing protein-protein interactions. These changes can be achieved in a multitude of ways. Reduction of expression level (or complete abolishment) can be achieved by swapping the native ribosome binding site (RBS) or promoter with another with lower strength/efficiency. ATG start sites can be swapped to a GTG, TTG, or CTG start codon, which results in reduction in translational activity of the coding region. Complete abolishment of expression can be done by knocking out (deleting) the coding region of a gene. Frameshifting the open reading frame (ORF) likely will result in a premature stop codon along the ORF, thereby creating a non-functional truncated product. Insertion of in-frame stop codons will also similarly create a non-functional truncated product. Addition of a degradation tag at the N or C terminal can also be done to reduce the effective concentration of a particular gene.

Conversely, expression level of the genes described herein can be achieved by using a stronger promoter. To ensure high promoter activity during high nitrogen level condition (or any other condition), a transcription profile of the whole genome in a high nitrogen level condition could be obtained and active promoters with a desired transcription level can be chosen from that dataset to replace the weak promoter. Weak start codons can be swapped out with an ATG start codon for better translation initiation efficiency. Weak ribosomal binding sites (RBS) can also be swapped out with a different RBS with higher translation initiation efficiency. In addition, site-specific mutagenesis can also be performed to alter the activity of an enzyme.

Increasing the level of nitrogen fixation that occurs in a plant can lead to a reduction in the amount of chemical fertilizer needed for crop production and reduce greenhouse gas emissions (e.g., nitrous oxide).

### Regulation of Colonization Potential

One trait that may be targeted for regulation by the methods described herein is colonization potential. Accordingly, in some embodiments, pathways and genes involved in colonization may act as a target for genetic engineering and optimization.

In some cases, exopolysaccharides may be involved in bacterial colonization of plants. In some cases, plant colonizing microbes may produce a biofilm. In some cases, plant colonizing microbes secrete molecules which may assist in adhesion to the plant, or in evading a plant immune response. In some cases, plant colonizing microbes may excrete signaling molecules which alter the plants response to the microbes. In some cases, plant colonizing microbes may secrete molecules which alter the local microenvironment. In some cases, a plant colonizing microbe may alter expression of genes to adapt to a plant said microbe is in proximity to. In some cases, a plant colonizing microbe may detect the presence of a plant in the local environment and may change expression of genes in response.

In some embodiments, to improve colonization, a gene involved in a pathway selected from the group consisting of: exopolysaccharide production, endo-polygalaturonase production, trehalose production, and glutamine conversion may be targeted for genetic engineering and optimization.

In some embodiments, an enzyme or pathway involved in production of exopolysaccharides may be genetically modified to improve colonization. Exemplary genes encoding an exopolysaccharide producing enzyme that may be targeted to improve colonization include, but are not limited to, bcsii, bcsiii, and yjbE.

In some embodiments, an enzyme or pathway involved in production of a filamentous hemagglutinin may be genetically modified to improve colonization. For example, a ƒhaB gene encoding a filamentous hemagglutinin may be targeted to improve colonization.

In some embodiments, an enzyme or pathway involved in production of an endo-polygalaturonase may be genetically modified to improve colonization. For example, a pehA gene encoding an endo-polygalaturonase precursor may be targeted to improve colonization.

In some embodiments, an enzyme or pathway involved in production of trehalose may be genetically modified to improve colonization. Exemplary genes encoding a trehalose producing enzyme that may be targeted to improve colonization include, but are not limited to, otsB and treZ.

In some embodiments, an enzyme or pathway involved in conversion of glutamine may be genetically modified to improve colonization. For example, the glsA2 gene encodes a glutaminase which converts glutamine into ammonium and glutamate. Upregulating glsA2 improves fitness by increasing the cell’s glutamate pool, thereby increasing available N to the cells. Accordingly, in some embodiments, the glsA2 gene may be targeted to improve colonization.

In some embodiments, colonization genes selected from the group consisting of: bcsii, bcsiii, yjbE, fhaB, pehA, otsB, treZ, glsA2, and combinations thereof, may be genetically modified to improve colonization.

Colonization genes that may be targeted to improve the colonization potential are also described in a PCT publication, WO/2019/032926, which is incorporated by reference herein in its entirety.

### Generation of Bacterial Populations

### Isolation of Bacteria

Microbes useful in methods and compositions disclosed herein can be obtained by extracting microbes from surfaces or tissues of native plants. Microbes can be obtained by grinding seeds to isolate microbes. Microbes can be obtained by planting seeds in diverse soil samples and recovering microbes from tissues. Additionally, microbes can be obtained by inoculating plants with exogenous microbes and determining which microbes appear in plant tissues. Non-limiting examples of plant tissues may include a seed, seedling, leaf, cutting, plant, bulb, or tuber.

A method of obtaining microbes may be through the isolation of bacteria from soils. Bacteria may be collected from various soil types. In some example, the soil can be characterized by traits such as high or low fertility, levels of moisture, levels of minerals, and various cropping practices. For example, the soil may be involved in a crop rotation where different crops are planted in the same soil in successive planting seasons. The sequential growth of different crops on the same soil may prevent disproportionate depletion of certain minerals. The bacteria can be isolated from the plants growing in the selected soils. The seedling plants can be harvested at 2-6 weeks of growth. For example, at least 400 isolates can be collected in a round of harvest. Soil and plant types reveal the plant phenotype as well as the conditions, which allow for the downstream enrichment of certain phenotypes.

Microbes can be isolated from plant tissues to assess microbial traits. The parameters for processing tissue samples may be varied to isolate different types of associative microbes, such as rhizospheric bacteria, epiphytes, or endophytes. The isolates can be cultured in nitrogen-free media to enrich for bacteria that perform nitrogen fixation. Alternatively, microbes can be obtained from global strain banks.

In planta analytics are performed to assess microbial traits. In some embodiments, the plant tissue can be processed for screening by high throughput processing for DNA and RNA. Additionally, non-invasive measurements can be used to assess plant characteristics, such as colonization. Measurements on wild microbes can be obtained on a plant-by-plant basis. Measurements on wild microbes can also be obtained in the field using medium throughput methods. Measurements can be done successively over time. Model plant system can be used including, but not limited to, Setaria.

Microbes in a plant system can be screened via transcriptional profiling of a microbe in a plant system. Examples of screening through transcriptional profiling are using methods of quantitative polymerase chain reaction (qPCR), molecular barcodes for transcript detection, Next Generation Sequencing, and microbe tagging with fluorescent markers. Impact factors can be measured to assess colonization in the greenhouse including, but not limited to, microbiome, abiotic factors, soil conditions, oxygen, moisture, temperature, inoculum conditions, and root localization. Nitrogen fixation can be assessed in bacteria by measuring 15N gas/fertilizer (dilution) with IRMS or NanoSIMS as described herein NanoSIMS is high-resolution secondary ion mass spectrometry. The NanoSIMS technique is a way to investigate chemical activity from biological samples. The catalysis of reduction of oxidation reactions that drive the metabolism of microorganisms can be investigated at the cellular, subcellular, molecular and elemental level. NanoSIMS can provide high spatial resolution of greater than 0.1 µm . NanoSIMS can detect the use of isotope tracers such as ¹³C, ¹⁵N, and ¹⁸O. Therefore, NanoSIMS can be used to the chemical activity nitrogen in the cell.

Automated greenhouses can be used for planta analytics. Plant metrics in response to microbial exposure include, but are not limited to, biomass, chloroplast analysis, CCD camera, volumetric tomography measurements.

One way of enriching a microbe population is according to genotype. For example, a polymerase chain reaction (PCR) assay with a targeted primer or specific primer. Primers designed for the nifH gene can be used to identity diazotrophs because diazotrophs express the nifH gene in the process of nitrogen fixation. A microbial population can also be enriched via single-cell culture-independent approaches and chemotaxis-guided isolation approaches. Alternatively, targeted isolation of microbes can be performed by culturing the microbes on selection media. Premeditated approaches to enriching microbial populations for desired traits can be guided by bioinformatics data and are described herein.

### Enriching for Microbes With Nitrogen Fixation Capabilities Using Bioinformatics

Bioinformatic tools can be used to identify and isolate plant growth promoting rhizobacteria (PGPRs), which are selected based on their ability to perform nitrogen fixation. Microbes with high nitrogen fixing ability can promote favorable traits in plants. Bioinformatic modes of analysis for the identification of PGPRs include, but are not limited to, genomics, metagenoinics, targeted isolation, gene sequencing, transcriptome sequencing, and modeling.

Genomics analysis can be used to identify PGPRs and confirm the presence of mutations with methods of Next Generation Sequencing as described herein and microbe version control.

Metagenomics can be used to identify and isolate PGPR. using a prediction algorithm for colonization. Metadata can also be used to identify the presence of an engineered strain in environmental and greenhouse samples.

Transcriptomic sequencing can be used to predict genotypes leading to PGPR phenotypes. Additionally, transcriptomic data is used to identify promoters for altering gene expression. Transcriptomic data can be analyzed in conjunction with the Whole Genome Sequence (WGS) to generate models of metabolism and gene regulatory networks.

### Domestication of Microbes

Microbes isolated from nature can undergo a domestication process wherein the microbes are converted to a form that is genetically trackable and identifiable. One way to domesticate a microbe is to engineer it with antibiotic resistance. The process of engineering antibiotic resistance can begin by determining the antibiotic sensitivity in the wild type microbial strain. If the bacteria are sensitive to the antibiotic, then the antibiotic can be a good candidate for antibiotic resistance engineering. Subsequently, an antibiotic resistant gene or a counterselectable suicide vector can be incorporated into the genome of a microbe using recombineering methods. A counterselectable suicide vector may consist of a deletion of the gene of interest, a selectable marker, and the counterselectable marker sacB. Counterselection can be used to exchange native microbial DNA sequences with antibiotic resistant genes. A medium throughput method can be used to evaluate multiple microbes simultaneously allowing for parallel domestication. Alternative methods of domestication include the use of homing nucleases to prevent the suicide vector sequences from looping out or from obtaining intervening vector sequences.

DNA vectors can be introduced into bacteria via several methods including electroporation and chemical transformations. A standard library of vectors can be used for transformations. An example of a method of gene editing is CRISPR preceded by Cas9 testing to ensure activity of Cas9 in the microbes.

### Engineering of Microbes

A microbial population with favorable traits can be obtained via directed evolution. Directed evolution is an approach wherein the process of natural selection is mimicked to evolve proteins or nucleic acids towards a user-defined goal. An example of directed evolution is when random mutations are introduced into a microbial population, the microbes with the most favorable traits are selected, and the growth of the selected microbes is continued. The most favorable traits in growth promoting rhizobacteria (PGPRs) may be in nitrogen fixation. The method of directed evolution may be iterative and adaptive based on the selection process after each iteration.

Plant growth promoting rhizobacteria (PGPRs) with high capability of nitrogen fixation can be generated. The evolution of PGPRs can be carried out via the introduction of genetic variation. Genetic variation can be introduced via polymerase chain reaction mutagenesis, oligonucleotide-directed mutagenesis, saturation mutagenesis, fragment shuffling mutagenesis, homologous recombination, CRISPR/Cas9 systems, chemical mutagenesis, and combinations thereof. These approaches can introduce random mutations into the microbial population. For example, mutants can be generated using synthetic DNA or RNA via oligonucleotide-directed mutagenesis. Mutants can be generated using tools contained on plasmids, which are later cured. Genes of interest can be identified using libraries from other species with improved traits including, but not limited to, improved PGPR properties, improved colonization of cereals, increased oxygen sensitivity, increased nitrogen fixation, and increased ammonia excretion. Intrageneric and intergeneric genes can be designed based on these libraries using software such as Geneious or Platypus design software. Mutations can be designed with the aid of machine learning. Mutations can be designed with the aid of a metabolic model. Automated design of the mutation can be done using a la Platypus and will guide RNAs for Cas-directed mutagenesis.

The intra-generic or intergeneric genes can be transferred into the host microbe. Additionally, reporter systems can also be transferred to the microbe. The reporter systems characterize promoters, determine the transformation success, screen mutants, and act as negative screening tools.

The microbes carrying the mutation can be cultured via serial passaging. A microbial colony contains a single variant of the microbe. Microbial colonies are screened with the aid of an automated colony picker and liquid handler. Mutants with gene duplication and increased copy number express a higher genotype of the desired trait.

### Selection of Plant Growth Promoting Microbes Based on Nitrogen Fixation

The microbial colonies can be screened using various assays to assess nitrogen fixation. One way to measure nitrogen fixation is via a single fermentative assay, which measures nitrogen excretion. An alternative method is the acetylene reduction assay (ARA) with in-line sampling over time. ARA can be performed in high throughput plates of microtube arrays. ARA can be performed with live plants and plant tissues. The media formulation and media oxygen concentration can be varied in ARA assays. Another method of screening microbial variants is by using biosensors. The use of NanoSIMS and Raman microspectroscopy can be used to investigate the activity of the microbes. In some cases, bacteria can also be cultured and expanded using methods of fermentation in bioreactors. The bioreactors are designed to improve robustness of bacteria growth and to decrease the sensitivity of bacteria to oxygen. Medium to high TP plate-based microfermentors are used to evaluate oxygen sensitivity, nutritional needs, nitrogen fixation, and nitrogen excretion. The bacteria can also be co-cultured with competitive or beneficial microbes to elucidate cryptic pathways. Flow cytometry can be used to screen for bacteria that produce high levels of nitrogen using chemical, colorimetric, or fluorescent indicators. The bacteria may be cultured in the presence or absence of a nitrogen source. For example, the bacteria may be cultured with glutamine, ammonia, urea or nitrates.

### Guided Microbial Remodeling - An Overview

Guided microbial remodeling is a method to systematically identify and improve the role of species within the crop microbioine. In some aspects, and according to a particular methodology of grouping/categorization, the method comprises three steps: 1) selection of candidate species by mapping plant-microbe interactions and predicting regulatory networks linked to a particular phenotype, 2) pragmatic and predictable improvement of microbial phenotypes through intra-species crossing of regulatory networks and gene clusters within a microbe’s genome, and 3) screening and selection of new microbial genotypes that produce desired crop phenotypes.

To systematically assess the improvement of strains, a model is created that links colonization dynamics of the microbial community to genetic activity by key species. The model is used to predict genetic targets for non-intergeneric genetic remodeling (i.e. engineering the genetic architecture of the microbe in a non-transgenic fashion). Rational improvement of the crop microbiome may be used to increase soil biodiversity, tune impact of keystone species, and/or alter timing and expression of important metabolic pathways.

To this end, the inventors have developed a platform to identify and improve the role of strains within the crop microbiome. In some aspects, the inventors call this process microbial breeding.

### Serial Passage

Production of bacteria to improve plant traits (e.g., nitrogen fixation) can be achieved through serial passage. The production of these bacteria can be done by selecting plants, which have a particular improved trait that is influenced by the microbial flora, in addition to identifying bacteria and/or compositions that are capable of imparting one or more improved traits to one or more plants. One method of producing a bacteria to improve a plant trait includes the steps of: (a) isolating bacteria from tissue or soil of a first plant; (b) introducing a genetic variation into one or more of the bacteria to produce one or more variant bacteria; (c) exposing a plurality of plants to the variant bacteria; (d) isolating bacteria from tissue or soil of one of the plurality of plants, wherein the plant from which the bacteria is isolated has an improved trait relative to other plants in the plurality of plants; and (e) repeating steps (b) to (d) with bacteria isolated from the plant with an improved trait (step (d)). Steps (b) to (d) can be repeated any number of times (e.g., once, twice, three times, four times, five times, ten times, or more) until the improved trait in a plant reaches a desired level. Further, the plurality of plants can be more than two plants, such as 10 to 20 plants, or 20 or more, 50 or more, 100 or more, 300 or more, 500 or more, or 1000 or more plants.

In addition to obtaining a plant with an improved trait, a bacterial population comprising bacteria comprising one or more genetic variations introduced into one or more genes (e.g., genes regulating nitrogen fixation) is obtained. By repeating the steps described above, a population of bacteria can be obtained that include the most appropriate members of the population that correlate with a plant trait of interest. The bacteria in this population can be identified and their beneficial properties determined, such as by genetic and/or phenotypic analysis. Genetic analysis may occur of isolated bacteria in step (a). Phenotypic and/or genotypic information may be obtained using techniques including: high through-put screening of chemical components of plant origin, sequencing techniques including high throughput sequencing of genetic material, differential display techniques (including DDRT-PCR, and DD-PCR), nucleic acid microarray techniques, RNA-sequencing (Whole Transcriptome Shotgun Sequencing), and qRT-PCR (quantitative real time PCR). Information gained can be used to obtain community profiling information on the identity and activity of bacteria present, such as phylogenetic analysis or microarray-based screening of nucleic acids coding for components of rRNA operons or other taxonomically informative loci. Examples of taxonomically informative loci include 16S rRNA gene, 23S rRNA gene, 5S rRNA gene, 5.8S rRNA gene, 12S rRNA gene, 18S rRNA gene, 28S rRNA gene, gyrB gene, rpoB gene, fusA gene, recA gene, coxl gene, nifD gene. Example processes of taxonomic profiling to determine taxa present in a population are described in US20140155283. Bacterial identification may comprise characterizing activity of one or more genes or one or more signaling pathways, such as genes associated with the nitrogen fixation pathway. Synergistic interactions (where two components, by virtue of their combination, increase a desired effect by more than an additive amount) between different bacterial species may also be present in the bacterial populations.

### Genetic Variation - Locations and Sources of Genomic Alteration

The genetic variation may be a gene selected from the group consisting of: nifA, nifL, ntrB, ntrC, glnA, glnB, gInK, draT, amtB, glnD, glnE, nifJ, nifH, nifD, nifK, nifY, nifE, nifN, nifU, nifS, nifV, nifW, nifZ, nifM, nifF, nifB, and nifQ. The genetic variation may be a variation in a gene encoding a protein with functionality selected from the group consisting of: glutamine synthetase, glutaminase, glutamine synthetase adenylyltransferase, transcriptional activator, anti-transcriptional activator, pyruvate flavodoxin oxidoreductase, flavodoxin, and NAD+-dinitrogen-reductase aDP-D-ribosyltransferase. The genetic variation may be a mutation that results in one or more of: increased expression or activity of NifA or glutaminase; decreased expression or activity of NifL, NtrB, glutamine synthetase, GInB, GInK, DraT, AmtB; decreased adenylyl-removing activity of GInE; or decreased uridylyl-removing activity of GInD. The genetic variation may be a variation in a gene selected from the group consisting of: bcsii, bcsiii, yjbE, fhaB, pehA, otsB, treZ, glsA2, and combinations thereof. In some embodiments, a genetic variation may be a variation in any of the genes described throughout this disclosure.

Introducing a genetic variation may comprise insertion and/or deletion of one or more nucleotides at a target site, such as 1, 2, 3, 4, 5, 10, 25, 50, 100, 250, 500, or more nucleotides. The genetic variation introduced into one or more bacteria of the methods disclosed herein may be a knock-out mutation (e.g. deletion of a promoter, insertion or deletion to produce a premature stop codon, deletion of an entire gene), or it may be elimination or abolishment of activity of a protein domain (e.g. point mutation affecting an active site, or deletion of a portion of a gene encoding the relevant portion of the protein product), or it may alter or abolish a regulatory sequence of a target gene. One or more regulatory sequences may also be inserted, including heterologous regulatory sequences and regulatory sequences found within a genome of a bacterial species or genus corresponding to the bacteria into which the genetic variation is introduced. Moreover, regulatory sequences may be selected based on the expression level of a gene in a bacterial culture or within a plant tissue. The genetic variation may be a predetermined genetic variation that is specifically introduced to a target site. The genetic variation may be a random mutation within the target site. The genetic variation may be an insertion or deletion of one or more nucleotides. In some cases, a plurality of different genetic variations (e.g. 2, 3, 4, 5, 10, or more) are introduced into one or more of the isolated bacteria before exposing the bacteria to plants for assessing trait improvement. The plurality of genetic variations can be any of the above types, the same or different types, and in any combination. In some cases, a plurality of different genetic variations are introduced serially, introducing a first genetic variation after a first isolation step, a second genetic variation after a second isolation step, and so forth so as to accumulate a plurality of genetic variations in bacteria imparting progressively improved traits on the associated plants.

### Genetic Variation — Methods of Introducing Genomic Alteration

In general, the term “genetic variation” refers to any change introduced into a polynucleotide sequence relative to a reference polynucleotide, such as a reference genome or portion thereof, or reference gene or portion thereof. A genetic variation may be referred to as a “mutation,” and a sequence or organism comprising a genetic variation may be referred to as a “genetic variant” or “mutant”. Genetic variations can have any number of effects, such as the increase or decrease of some biological activity, including gene expression, metabolism, and cell signaling. Genetic variations can be specifically introduced to a target site, or introduced randomly. A variety of molecular tools and methods are available for introducing genetic variation. For example, genetic variation can be introduced via polymerase chain reaction mutagenesis, oligonucleotide-directed mutagenesis, saturation mutagenesis, fragment shuffling mutagenesis, homologous recombination, recombineering, lambda red mediated recombination, CRISPR/Cas9 systems, chemical mutagenesis, and combinations thereof. Chemical methods of introducing genetic variation include exposure of DNA to a chemical mutagen, e.g., ethyl methanesulfonate (EMS), methyl methanesulfonate (MMS), N-nitrosourea (EN U), N-methyl-N-nitro-N′-nitrosoguanidine, 4-nitroquinoline N-oxide, diethylsulfate, benzopyrene, cyclophosphamide, bleomycin, triethylmelamine, acrylamide monomer, nitrogen mustard, vincristine, diepoxyalkanes (for example, diepoxybutane), ICR-170, formaldehyde, procarbazine hydrochloride, ethylene oxide, dimethylnitrosamine, 7,12 dimethylbenz(a)anthracene, chlorambucil, hexamethylphosphoramide, bisulfan, and the like. Radiation mutation-inducing agents include ultraviolet radiation, γ-irradiation, X-rays, and fast neutron bombardment. Genetic variation can also be introduced into a nucleic acid using, e.g., trimethylpsoralen with ultraviolet light. Random or targeted insertion of a mobile DNA element, e.g., a transposable element, is another suitable method for generating genetic variation. Genetic variations can be introduced into a nucleic acid during amplification in a cell-free in vitro system, e.g., using a polymerase chain reaction (PCR) technique such as error-prone PCR. Genetic variations can be introduced into a nucleic acid in vitro using DNA shuffling techniques (e.g., exon shuffling, domain swapping, and the like). Genetic variations can also be introduced into a nucleic acid as a result of a deficiency in a DNA repair enzyme in a cell, e.g., the presence in a cell of a mutant gene encoding a mutant DNA repair enzyme is expected to generate a high frequency of mutations (i.e., about 1 mutation/100 genes-1 mutation/10,000 genes) in the genome of the cell. Examples of genes encoding DNA repair enzymes include but are not limited to Mut H, Mut S, Mut L, and Mut U, and the homologs thereof in other species (e.g., MSH 1 6, PMS 1 2, MLH 1, GTBP, ERCC-1, and the like). Example descriptions of various methods for introducing genetic variations are provided in e.g., Stemple (2004) Nature 5:1-7; Chiang et al. (1993) PCR Methods Appl 2(3): 210-217; Stemmer (1994) Proc. Natl. Acad. Sci. USA 91:10747-10751; and U.S. Pat. Nos. 6,033,861, and 6,773,900.

Genetic variations introduced into microbes may be classified as transgenic, cisgenic, intragenomic, intrageneric, intergeneric, synthetic, evolved, rearranged, or SNPs.

Genetic variation may be introduced into numerous metabolic pathways within microbes to elicit improvements in the traits described above. Representative pathways include sulfur uptake pathways, glycogen biosynthesis, the glutamine regulation pathway, the molybdenum uptake pathway, the nitrogen fixation pathway, ammonia assimilation, ammonia excretion or secretion, Nitrogen uptake, glutamine biosynthesis, colonization pathways, annamox, phosphate solubilization, organic acid transport, organic acid production, agglutinins production, reactive oxygen radical scavenging genes, Indole Acetic Acid biosynthesis, trehalose biosynthesis, plant cell wall degrading enzymes or pathways, root attachment genes, exopolysaccharide secretion, glutamate synthase pathway, iron uptake pathways, siderophore pathway, chitinase pathway, ACC deaminase, glutathione biosynthesis, phosphorous signaling genes, quorum quenching pathway, cytochrome pathways, hemoglobin pathway, bacterial hemoglobin-like pathway, small RNA rsmZ, rhizobitoxine biosynthesis, lapA adhesion protein, AHL quorum sensing pathway, phenazine biosynthesis, cyclic lipopeptide biosynthesis, and antibiotic production.

CRISPR/Cas9 (Clustered regularly interspaced short palindromic repeats) /CRISPR-associated (Cas) systems can be used to introduce desired mutations. CRISPR/Cas9 provide bacteria and archaea with adaptive immunity against viruses and plasmids by using CRISPR RNAs (crRNAs) to guide the silencing of invading nucleic acids. The Cas9 protein (or functional equivalent and/or variant thereof, i.e., Cas9-like protein) naturally contains DNA endonuclease activity that depends on the association of the protein with two naturally occurring or synthetic RNA molecules called crRNA and tracrRNA (also called guide RNAs). In some cases, the two molecules are covalently link to form a single molecule (also called a single guide RNA (“sgRNA”). Thus, the Cas9 or Cas9-like protein associates with a DNA-targeting RNA (which term encompasses both the two-molecule guide RNA configuration and the single-molecule guide RNA configuration), which activates the Cas9 or Cas9-like protein and guides the protein to a target nucleic acid sequence. If the Cas9 or Cas9-like protein retains its natural enzymatic function, it will cleave target DNA to create a double-stranded break, which can lead to genome alteration (i.e., editing: deletion, insertion (when a donor polynucleotide is present), replacement, etc.), thereby altering gene expression. Some variants of Cas9 (which variants are encompassed by the term Cas9-like) have been altered such that they have a decreased DNA cleaving activity (in some cases, they cleave a single strand instead of both strands of the target DNA, while in other cases, they have severely reduced to no DNA cleavage activity). Further exemplary descriptions of CRISPR systems for introducing genetic variation can be found in, e.g. US8795965.

As a cyclic amplification technique, polymerase chain reaction (PCR) mutagenesis uses mutagenic primers to introduce desired mutations. PCR is performed by cycles of denaturation, annealing, and extension. After amplification by PCR, selection of mutated DNA and removal of parental plasmid DNA can be accomplished by: 1) replacement of dCTP by hydroxymethylated-dCTP during PCR, followed by digestion with restriction enzymes to remove non-hydroxymethylated parent DNA only; 2) simultaneous mutagenesis of both an antibiotic resistance gene and the studied gene changing the plasmid to a different antibiotic resistance, the new antibiotic resistance facilitating the selection of the desired mutation thereafter; 3) after introducing a desired mutation, digestion of the parent methylated template DNA by restriction enzyme Dpnl which cleaves only methylated DNA by which the mutagenized unmethylated chains are recovered; or 4) circularization of the mutated PCR products in an additional ligation reaction to increase the transformation efficiency of mutated DNA. Further description of exemplary methods can be found in e.g. US7132265, US6713285, US6673610, US6391548, US5789166, US5780270, US5354670, US5071743, and US20100267147.

Oligonucleotide-directed mutagenesis, also called site-directed mutagenesis, typically utilizes a synthetic DNA primer. This synthetic primer contains the desired mutation and is complementary to the template DNA around the mutation site so that it can hybridize with the DNA in the gene of interest. The mutation may be a single base change (a point mutation), multiple base changes, deletion, or insertion, or a combination of these. The single-strand primer is then extended using a DNA polymerase, which copies the rest of the gene. The gene thus copied contains the mutated site, and may then be introduced into a host cell as a vector and cloned. Finally, mutants can be selected by DNA sequencing to check that they contain the desired mutation.

Genetic variations can be introduced using error-prone PCR. In this technique the gene of interest is amplified using a DNA polymerase under conditions that are deficient in the fidelity of replication of sequence. The result is that the amplification products contain at least one error in the sequence. When a gene is amplified and the resulting product(s) of the reaction contain one or more alterations in sequence when compared to the template molecule, the resulting products are mutagenized as compared to the template. Another means of introducing random mutations is exposing cells to a chemical mutagen, such as nitrosoguanidine or ethyl methanesulfonate (Nestmann, Mutat Res 1975 June; 28(3):323-30), and the vector containing the gene is then isolated from the host.

Saturation mutagenesis is another form of random mutagenesis, in which one tries to generate all or nearly all possible mutations at a specific site, or narrow region of a gene. In a general sense, saturation mutagenesis is comprised of mutagenizing a complete set of mutagenic cassettes (wherein each cassette is, for example, 1-500 bases in length) in defined polynucleotide sequence to be mutagenized (wherein the sequence to be mutagenized is, for example, from 15 to 100, 000 bases in length). Therefore, a group of mutations (e.g. ranging from 1 to 100 mutations) is introduced into each cassette to be mutagenized. A grouping of mutations to be introduced into one cassette can be different or the same from a second grouping of mutations to be introduced into a second cassette during the application of one round of saturation mutagenesis. Such groupings are exemplified by deletions, additions, groupings of particular codons, and groupings of particular nucleotide cassettes.

Fragment shuffling mutagenesis, also called DNA shuffling, is a way to rapidly propagate beneficial mutations. In an example of a shuffling process, DNAse is used to fragment a set of parent genes into pieces of e.g. about 50-100 bp in length. This is then followed by a polymerase chain reaction (PCR) without primers--DNA fragments with sufficient overlapping homologous sequence will anneal to each other and are then be extended by DNA polymerase. Several rounds of this PCR extension are allowed to occur, after some of the DNA molecules reach the size of the parental genes. These genes can then be amplified with another PCR, this time with the addition of primers that are designed to complement the ends of the strands. The primers may have additional sequences added to their 5′ ends, such as sequences for restriction enzyme recognition sites needed for ligation into a cloning vector. Further examples of shuffling techniques are provided in US20050266541.

Homologous recombination mutagenesis involves recombination between an exogenous DNA fragment and the targeted polynucleotide sequence. After a double-stranded break occurs, sections of DNA around the 5′ ends of the break are cut away in a process called resection. In the strand invasion step that follows, an overhanging 3′ end of the broken DNA molecule then “invades” a similar or identical DNA molecule that is not broken. The method can be used to delete a gene, remove exons, add a gene, and introduce point mutations. Homologous recombination mutagenesis can be permanent or conditional. Typically, a recombination template is also provided. A recombination template may be a component of another vector, contained in a separate vector, or provided as a separate polynucleotide. In some embodiments, a recombination template is designed to serve as a template in homologous recombination, such as within or near a target sequence nicked or cleaved by a site-specific nuclease. A template polynucleotide may be of any suitable length, such as about or more than about 10, 15, 20, 25, 50, 75, 100, 150, 200, 500, 1000, or more nucleotides in length. In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence. Non-limiting examples of site-directed nucleases useful in methods of homologous recombination include zinc finger nucleases, CRISPR nucleases, TALE nucleases, and meganuclease. For a further description of the use of such nucleases, see e.g. US8795965 and US20140301990.

Mutagens that create primarily point mutations and short deletions, insertions, transversions, and/or transitions, including chemical mutagens or radiation, may be used to create genetic variations. Mutagens include, but are not limited to, ethyl methanesulfonate, methylmethane sulfonate, N-ethyl-N-nitrosurea, triethylmelamine, N-methyl-N-nitrosourea, procarbazine, chlorambucil, cyclophosphamide, diethyl sulfate, acrylamide monomer, melphalan, nitrogen mustard, vincristine, dimethylnitrosamine, N-methyl-N′-nitro-Nitrosoguanidine, nitrosoguanidine, 2-aminopurine, 7,12 dimethyl-benz(a)anthracene, ethylene oxide, hexamethylphosphoramide, bisulfan, diepoxyalkanes (diepoxyoctane, diepoxybutane, and the like), 2-methoxy-6-chloro-9[3-(ethyl-2-chloroethyl)aminopropylamino]acridine dihydrochloride and formaldehyde.

Introducing genetic variation may be an incomplete process, such that some bacteria in a treated population of bacteria carry a desired mutation while others do not. In some cases, it is desirable to apply a selection pressure so as to enrich for bacteria carrying a desired genetic variation. Traditionally, selection for successful genetic variants involved selection for or against some functionality imparted or abolished by the genetic variation, such as in the case of inserting antibiotic resistance gene or abolishing a metabolic activity capable of converting a non-lethal compound into a lethal metabolite. It is also possible to apply a selection pressure based on a polynucleotide sequence itself, such that only a desired genetic variation need be introduced (e.g. without also requiring a selectable marker). In this case, the selection pressure can comprise cleaving genomes lacking the genetic variation introduced to a target site, such that selection is effectively directed against the reference sequence into which the genetic variation is sought to be introduced. Typically, cleavage occurs within 100 nucleotides of the target site (e.g. within 75, 50, 25, 10, or fewer nucleotides from the target site, including cleavage at or within the target site). Cleaving may be directed by a site-specific nuclease selected from the group consisting of a Zinc Finger nuclease, a CRISPR nuclease, a TALE nuclease (TALEN), and a meganuclease. Such a process is similar to processes for enhancing homologous recombination at a target site, except that no template for homologous recombination is provided. As a result, bacteria lacking the desired genetic variation are more likely to undergo cleavage that, left unrepaired, results in cell death. Bacteria surviving selection may then be isolated for use in exposing to plants for assessing conferral of an improved trait.

A CRISPR nuclease may be used as the site-specific nuclease to direct cleavage to a target site. An improved selection of mutated microbes can be obtained by using Cas9 to kill non-mutated cells. Plants are then inoculated with the mutated microbes to re-confirm symbiosis and create evolutionary pressure to select for efficient symbionts. Microbes can then be re-isolated from plant tissues. CRISPR nuclease systems employed for selection against non-variants can employ similar elements to those described above with respect to introducing genetic variation, except that no template for homologous recombination is provided. Cleavage directed to the target site thus enhances death of affected cells.

Other options for specifically inducing cleavage at a target site are available, such as zinc finger nucleases, TALE nuclease (TALEN) systems, and meganuclease. Zinc-finger nucleases (ZFNs) are artificial DNA endonucleases generated by fusing a zinc finger DNA binding domain to a DNA cleavage domain. ZFNs can be engineered to target desired DNA sequences and this enables zinc-finger nucleases to cleave unique target sequences. When introduced into a cell, ZFNs can be used to edit target DNA in the cell (e.g., the cell’s genome) by inducing double stranded breaks. Transcription activator-like effector nucleases (TALENs) are artificial DNA endonucleases generated by fusing a TAL (Transcription activator-like) effector DNA binding domain to a DNA cleavage domain. TALENS can be quickly engineered to bind practically any desired DNA sequence and when introduced into a cell, TALENs can be used to edit target DNA in the cell (e.g., the cell’s genome) by inducing double strand breaks. Meganucleases (homing endonuclease) are endodeoxyribonucleases characterized by a large recognition site (double-stranded DNA sequences of 12 to 40 base pairs. Meganucleases can be used to replace, eliminate or modify sequences in a highly targeted way. By modifying their recognition sequence through protein engineering, the targeted sequence can be changed. Meganucleases can be used to modify all genome types, whether bacterial, plant or animal and are commonly grouped into four families: the LAGLIDADG family (SEQ ID NO: 1), the GIY-YIG family, the His-Cyst box family and the HNH family. Exemplary homing endonucleases include I-SceI, I-CeuI, PI-PspI, PI-Sce, I-SceIV, I-CsmI, I-PanI, I-SceII, I-PpoI, I-ScellI, I-CreI, I-TevI, I-TevII and I-TevIII.

### Genetic Variation - Methods of Identification

The microbes of the present disclosure may be identified by one or more genetic modifications or alterations, which have been introduced into said microbe. One method by which said genetic modification or alteration can be identified is via reference to a SEQ ID NO that contains a portion of the microbe’s genomic sequence that is sufficient to identify the genetic modification or alteration.

Further, in the case of microbes that have not had a genetic modification or alteration (e.g. a wild type, WT) introduced into their genomes, the disclosure can utilize 16S nucleic acid sequences to identify said microbes. A 16S nucleic acid sequence is an example of a “molecular marker” or “genetic marker,” which refers to an indicator that is used in methods for visualizing differences in characteristics of nucleic acid sequences. Examples of other such indicators are restriction fragment length polymorphism (RFLP) markers, amplified fragment length polymorphism (AFLP) markers, single nucleotide polymorphisms (SNPs), insertion mutations, microsatellite markers (SSRs), sequence-characterized amplified regions (SCARs), cleaved amplified polymorphic sequence (CAPS) markers or isozyme markers or combinations of the markers described herein which defines a specific genetic and chromosomal location. Markers further include polynucleotide sequences encoding 16S or 18S rRNA, and internal transcribed spacer (ITS) sequences, which are sequences found between small-subunit and large-subunit rRNA genes that have proven to be especially useful in elucidating relationships or distinctions when compared against one another. Furthermore, the disclosure utilizes unique sequences found in genes of interest (e.g. nif H,D,K,L,A, glnE, amtB, etc.) to identify microbes disclosed herein.

The primary structure of major rRNA subunit 16S comprise a particular combination of conserved, variable, and hypervariable regions that evolve at different rates and enable the resolution of both very ancient lineages such as domains, and more modern lineages such as genera. The secondary structure of the 16S subunit include approximately 50 helices which result in base pairing of about 67% of the residues. These highly conserved secondary structural features are of great functional importance and can be used to ensure positional homology in multiple sequence alignments and phylogenetic analysis. Over the previous few decades, the 16S rRNA gene has become the most sequenced taxonomic marker and is the cornerstone for the current systematic classification of bacteria and archaea (Yarza et al. 2014. Nature Rev. Micro. 12:635-45).

### Improvement of Traits

Methods of the present disclosure may be employed to introduce or improve one or more of a variety of desirable traits. Examples of traits that may introduced or improved include: root biomass, root length, height, shoot length, leaf number, water use efficiency, overall biomass, yield, fruit size, grain size, photosynthesis rate, tolerance to drought, heat tolerance, salt tolerance, resistance to nematode stress, resistance to a fungal pathogen, resistance to a bacterial pathogen, resistance to a viral pathogen, level of a metabolite, and proteome expression. The desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the improved traits) grown under identical conditions.

A preferred trait to be introduced or improved is nitrogen fixation, as described herein. A second preferred trait to be introduced or improved is colonization potential, as described herein. In some cases, a plant resulting from the methods described herein exhibits a difference in the trait that is at least about 5% greater, for example at least about 5%, at least about 8%, at least about 10%, at least about 15%, at least about 20%, at least about 25%, at least about 30%, at least about 40%, at least about 50%, at least about 60%, at least about 75%, at least about 80%, at least about 80%, at least about 90%, or at least 100%, at least about 200%, at least about 300%, at least about 400% or greater than a reference agricultural plant grown under the same conditions in the soil. In additional examples, a plant resulting from the methods described herein exhibits a difference in the trait that is at least about 5% greater, for example at least about 5%, at least about 8%, at least about 10%, at least about 15%, at least about 20%, at least about 25%, at least about 30%, at least about 40%, at least about 50%, at least about 60%, at least about 75%, at least about 80%, at least about 80%, at least about 90%, or at least 100%, at least about 200%, at least about 300%, at least about 400% or greater than a reference agricultural plant grown under similar conditions in the soil.

The trait to be improved may be assessed under conditions including the application of one or more biotic or abiotic stressors. Examples of stressors include abiotic stresses (such as heat stress, salt stress, drought stress, cold stress, and low nutrient stress) and biotic stresses (such as nematode stress, insect herbivory stress, fungal pathogen stress, bacterial pathogen stress, and viral pathogen stress).

The trait improved by methods and compositions of the present disclosure may be nitrogen fixation, including in a plant not previously capable of nitrogen fixation. In some cases, bacteria isolated according to a method described herein produce 1% or more (e.g. 2%, 3%, 4%, 5%, 6%, 7%, 8%, 9%, 10%, 15%, 20%, or more) of a plant’s nitrogen, which may represent an increase in nitrogen fixation capability of at least 2-fold (e.g. 3-fold, 4-fold, 5-fold, 6-fold, 7-fold., 8-fold, 9-fold, 10-fold, 20-fold, 50-fold, 100-fold, 1000-fold, or more) as compared to bacteria isolated from the first plant before introducing any genetic variation. In some cases, the bacteria produce 5% or more of a plant’s nitrogen. The desired level of nitrogen fixation may be achieved after repeating the steps of introducing genetic variation, exposure to a plurality of plants, and isolating bacteria from plants with an improved trait one or more times (e.g. 1, 2, 3, 4, 5, 10, 15, 25, or more times). In some cases, enhanced levels of nitrogen fixation are achieved in the presence of fertilizer supplemented with glutamine, ammonia, or other chemical source of nitrogen. Methods for assessing degree of nitrogen fixation are known, examples of which are described herein.

### Measuring Nitrogen Delivered in an Agriculturally Relevant Field Context

In the field, the amount of nitrogen delivered can be determined by the function of colonization multiplied by the activity.

$$\text{Nitrogen} \text{delivered} = {\int }_{\text{Time} \& \text{Space}} \text{Colonization} \text{x} \text{Activity}$$

The above equation requires (1) the average colonization per unit of plant tissue, and (2) the activity as either the amount of nitrogen fixed or the amount of ammonia excreted by each microbial cell. To convert to pounds of nitrogen per acre, corn growth physiology is tracked over time, e.g., size of the plant and associated root system throughout the maturity stages.

The pounds of nitrogen delivered to a crop per acre-season can be calculated by the following equation:

$$\begin{matrix}\text{Nitrogen} \text{delivered} = \\ \int \text{Plant} \text{Tissue} \left(\text{t}\right) \times \text{Colonization} \left(\text{t}\right) \times \text{Activity} \left(\text{t}\right) \underline{\text{dt}}\end{matrix}$$

The Plant Tissue(t) is the fresh weight of corn plant tissue over the growing time (t). Values for reasonably making the calculation are described in detail in the publication entitled Roots, Growth and Nutrient Uptake (Mengel. Dept. of Agronomy Pub.# AGRY-95-08 (Rev. May-95. p. 1-8.).

The Colonization (t) is the amount of the microbes of interest found within the plant tissue, per gram fresh weight of plant tissue, at any particular time, t, during the growing season. In the instance of only a single timepoint available, the single timepoint is normalized as the peak colonization rate over the season, and the colonization rate of the remaining timepoints are adjusted accordingly.

Activity(t) is the rate of which N is fixed by the microbes of interest per unit time, at any particular time, t, during the growing season. In the embodiments disclosed herein, this activity rate is approximated by in vitro acetylene reduction assay (ARA) in ARA media in the presence of 5 mM glutamine or Ammonium excretion assay in ARA media in the presence of 5 mM ammonium ions.

The Nitrogen delivered amount is then calculated by numerically integrating the above function. In cases where the values of the variables described above are discretely measured at set timepoints, the values in between those timepoints are approximated by performing linear interpolation.

### Nitrogen Fixation

Described herein are methods of increasing nitrogen fixation in a plant, comprising exposing the plant to bacteria comprising one or more genetic variations introduced into one or more genes regulating nitrogen fixation, wherein the bacteria produce 1% or more of nitrogen in the plant (e.g. 2%, 5%, 10%, or more), which may represent a nitrogen-fixation capability of at least 2-fold as compared to the plant in the absence of the bacteria. The bacteria may produce the nitrogen in the presence of fertilizer supplemented with glutamine, urea, nitrates or ammonia. Genetic variations can be any genetic variation described herein, including examples provided above, in any number and any combination. The genetic variation may be introduced into a gene selected from the group consisting of nifA, nifL, ntrB, ntrC, glutamine synthetase, glnA, glnB, glnK, draT, amtB, glutaminase, glnD, glnE, nifJ, nifH, nifD, nifK, nifY, nifE, nifN, nifU, nifS, nifV, nifW, nifZ, nifM, nifF, nifB, and nifQ. The genetic variation may be a mutation that results in one or more of: increased expression or activity of nifA or glutaminase; decreased expression or activity of nifL, ntrB, glutamine synthetase, glnB, glnK, draT, amtB; decreased adenylyl-removing activity of GlnE; or decreased uridylyl-removing activity of GlnD. The genetic variation introduced into one or more bacteria of the methods disclosed herein may be a knock-out mutation or it may abolish a regulatory sequence of a target gene, or it may comprise insertion of a heterologous regulatory sequence, for example, insertion of a regulatory sequence found within the genome of the same bacterial species or genus. The regulatory sequence can be chosen based on the expression level of a gene in a bacterial culture or within plant tissue. The genetic variation may be produced by chemical mutagenesis. The plants grown in step (c) may be exposed to biotic or abiotic stressors.

In some embodiments, remodeled bacteria of the present disclosure each produce fixed N of at least about 2 × 10⁻¹³ mmol of N per CFU per hour, about 2.5 × 10⁻¹³ mmol of N per CFU per hour, about 3 × 10⁻¹³ mmol of N per CFU per hour, about 3.5 × 10⁻¹³ mmol of N per CFU per hour, about 4 × 10⁻¹³ mmol of N per CFU per hour, about 4.5 × 10⁻¹³ mmol of N per CFU per hour, about 5 × 10⁻¹³ mmol of N per CFU per hour, about 5.5 × 10⁻¹³ mmol of N per CFU per hour, about 6 × 10⁻¹³ mmol of N per CFU per hour, about 6.5 × 10⁻¹³ mmol of N per CFU per hour, about 7 × 10⁻¹³ mmol of N per CFU per hour, about 7.5 × 10⁻¹³ mmol of N per CFU per hour, about 8 × 10⁻¹³ mmol of N per CFU per hour, about 8.5 × 10⁻¹³ mmol of N per CFU per hour, about 9 × 10⁻¹³ mmol of N per CFU per hour, about 9.5 × 10⁻¹³ mmol of N per CFU per hour, or about 10 × 10⁻¹³ mmol of N per CFU per hour.

In some embodiments, remodeled bacteria of the present disclosure each produce fixed N of at least about 2 × 10⁻¹² mmol of N per CFU per hour, about 2.25 × 10⁻¹² mmol of N per CFU per hour, about 2.5 × 10⁻¹² mmol of N per CFU per hour, about 2.75 × 10⁻¹² mmol of N per CFU per hour, about 3 × 10⁻¹² mmol of N per CFU per hour, about 3.25 × 10⁻¹² mmol of N per CFU per hour, about 3.5 × 10⁻¹² mmol of N per CFU per hour, about 3.75 × 10⁻¹² mmol of N per CFU per hour, about 4 × 10⁻¹² mmol of N per CFU per hour, about 4.25 × 10⁻¹² mmol of N per CFU per hour, about 4.5 × 10⁻¹² mmol of N per CFU per hour, about 4.75 × 10⁻¹² mmol of N per CFU per hour, about 5 × 10⁻¹² mmol of N per CFU per hour, about 5.25 × 10⁻¹² mmol of N per CFU per hour, about 5.5 × 10⁻¹² mmol of N per CFU per hour, about 5.75 × 10⁻¹² mmol of N per CFU per hour, about 6 × 10⁻¹² mmol of N per CFU per hour, about 6.25 × 10⁻¹² mmol of N per CFU per hour, about 6.5 × 10⁻¹² mmol of N per CFU per hour, about 6.75 × 10⁻ ¹² mmol of N per CFU per hour, about 7 × 10⁻¹² mmol of N per CFU per hour, about 7.25 × 10⁻ ¹² mmol of N per CFU per hour, about 7.5 × 10⁻¹² mmol of N per CFU per hour, about 7.75 × 10⁻¹² mmol of N per CFU per hour, about 8 × 10⁻¹² mmol of N per CFU per hour, about 8.25 × 10⁻¹² mmol of N per CFU per hour, about 8.5 × 10⁻¹² mmol of N per CFU per hour, about 8.75 × 10⁻¹² mmol of N per CFU per hour, about 9 × 10⁻¹² mmol of N per CFU per hour, about 9.25 × 10⁻¹² mmol of N per CFU per hour, about 9.5 × 10⁻¹² mmol of N per CFU per hour, about 9.75 × 10⁻¹² mmol of N per CFU per hour, or about 10 × 10⁻¹² mmol of N per CFU per hour.

In some embodiments, remodeled bacteria of the present disclosure each produce fixed N of at least about 5.49 × 10⁻¹³ mmol of N per CFU per hour. In some embodiments, remodeled bacteria of the present disclosure produce fixed N of at least about 4.03 × 10⁻¹³ mmol of N per CFU per hour. In some embodiments, remodeled bacteria of the present disclosure produce fixed N of at least about 2.75 × 10⁻¹² mmol of N per CFU per hour.

In some embodiments, remodeled bacteria of the present disclosure in aggregate produce at least about 15 pounds of fixed N per acre, at least about 20 pounds of fixed N per acre, at least about 25 pounds of fixed N per acre, at least about 30 pounds of fixed N per acre, at least about 35 pounds of fixed N per acre, at least about 40 pounds of fixed N per acre, at least about 45 pounds of fixed N per acre, at least about 50 pounds of fixed N per acre, at least about 55 pounds of fixed N per acre, at least about 60 pounds of fixed N per acre, at least about 65 pounds of fixed N per acre, at least about 70 pounds of fixed N per acre, at least about 75 pounds of fixed N per acre, at least about 80 pounds of fixed N per acre, at least about 85 pounds of fixed N per acre, at least about 90 pounds of fixed N per acre, at least about 95 pounds of fixed N per acre, or at least about 100 pounds of fixed N per acre.

In some embodiments, remodeled bacteria of the present disclosure produce fixed N in the amounts disclosed herein over the course of at least about day 0 to about 80 days, at least about day 0 to about 70 days, at least about day 0 to about 60 days, at least about 1 day to about 80 days, at least about 1 day to about 70 days, at least about 1 day to about 60 days, at least about 2 days to about 80 days, at least about 2 days to about 70 days, at least about 2 days to about 60 days, at least about 3 days to about 80 days, at least about 3 days to about 70 days, at least about 3 days to about 60 days, at least about 4 days to about 80 days, at least about 4 days to about 70 days, at least about 4 days to about 60 days, at least about 5 days to about 80 days, at least about 5 days to about 70 days, at least about 5 days to about 60 days, at least about 6 days to about 80 days, at least about 6 days to about 70 days, at least about 6 days to about 60 days, at least about 7 days to about 80 days, at least about 7 days to about 70 days, at least about 7 days to about 60 days, at least about 8 days to about 80 days, at least about 8 days to about 70 days, at least about 8 days to about 60 days, at least about 9 days to about 80 days, at least about 9 days to about 70 days, at least about 9 days to about 60 days, at least about 10 days to about 80 days, at least about 10 days to about 70 days, at least about 10 days to about 60 days, at least about 15 days to about 80 days, at least about 15 days to about 70 days, at least about 15 days to about 60 days, at least about 20 days to about 80 days, at least about 20 days to about 70 days, or at least about 20 days to about 60 days.

In some embodiments, remodeled bacteria of the present disclosure produce fixed N in any of the amounts disclosed herein over the course of at least about 80 days ± 5 days, at least about 80 days ± 10 days, at least about 80 days ± 15 days, at least about 80 days ± 20 days, at least about 75 days ± 5 days, at least about 75 days ± 10 days, at least about 75 days ± 15 days, at least about 75 days ± 20 days, at least about 70 days ± 5 days, at least about 70 days ± 10 days, at least about 70 days ± 15 days, at least about 70 days ± 20 days, at least about 60 days ± 5 days, at least about 60 days ± 10 days, at least about 60 days ± 15 days, at least about 60 days ± 20 days.

In some embodiments, remodeled bacteria of the present disclosure produce fixed N in any of the amounts disclosed herein over the course of at least about 10 days to about 80 days, at least about 10 days to about 70 days, or at least about 10 days to about 60 days.

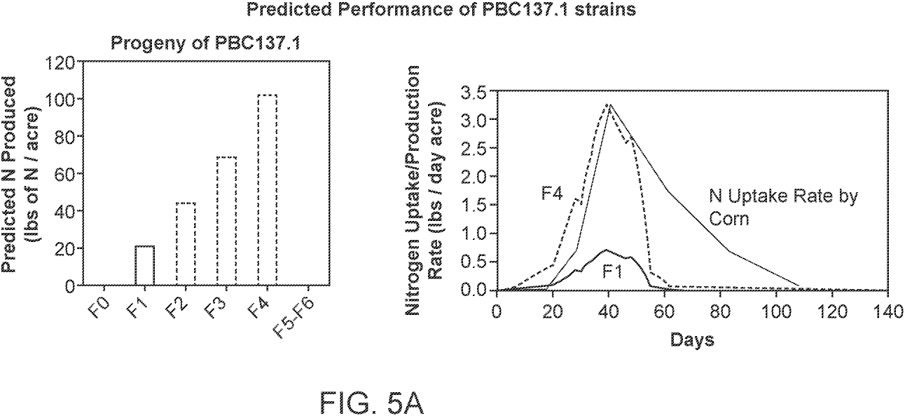

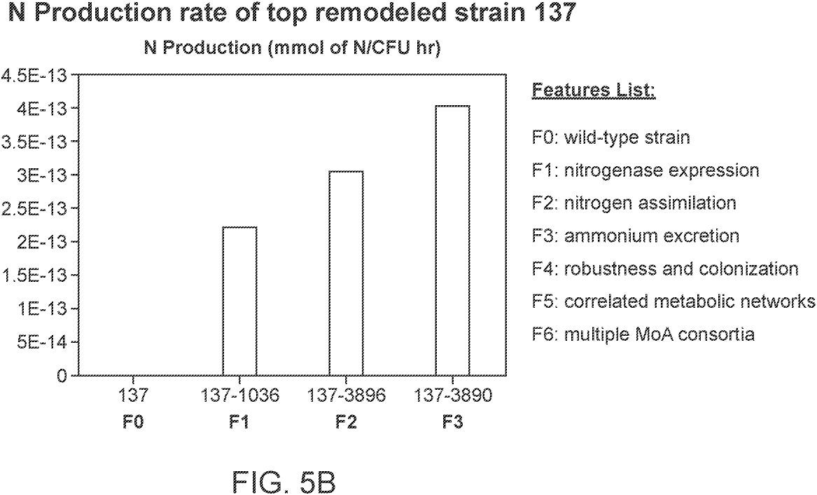

In some embodiments, remodeled bacteria of the present disclosure produce fixed N in the amounts and time shown in FIG. 5A, right panel.

The amount of nitrogen fixation that occurs in the plants described herein may be measured in several ways, for example by an acetylene-reduction (AR) assay. An acetylene-reduction assay can be performed in vitro or in vivo. Evidence that a particular bacterium is providing fixed nitrogen to a plant can include: 1) total plant N significantly increases upon inoculation, preferably with a concomitant increase in N concentration in the plant; 2) nitrogen deficiency symptoms are relieved under N-limiting conditions upon inoculation (which should include an increase in dry matter); 3) N₂ fixation is documented through the use of an ¹⁵N approach (which can be isotope dilution experiments, ¹⁵N₂ reduction assays, or ¹⁵N natural abundance assays); 4) fixed N is incorporated into a plant protein or metabolite; and 5) all of these effects are not be seen in non-inoculated plants or in plants inoculated with a mutant of the inoculum strain.

### Bacterial Species

Microbes useful in the methods and compositions disclosed herein may be obtained from any source. In some cases, microbes may be bacteria, archaea, protozoa or fungi. The microbes of this disclosure may be nitrogen fixing microbes, for example a nitrogen fixing bacteria, nitrogen fixing archaea, nitrogen fixing fungi, nitrogen fixing yeast, or nitrogen fixing protozoa. Microbes useful in the methods and compositions disclosed herein may be spore forming microbes, for example spore forming bacteria. In some cases, bacteria useful in the methods and compositions disclosed herein may be Gram positive bacteria or Gram negative bacteria. In some cases, the bacteria may be an endospore forming bacteria of the Firmicute phylum. In some cases, the bacteria may be a diazotroph. In some cases, the bacteria may not be a diazotroph.

The methods and compositions of this disclosure may be used with an archaea, such as, for example, Methanothermobacter thermoautotrophicus.

In some cases, bacteria which may be useful include, but are not limited to, Agrobacterium radiobacter, Bacillus acidocaldarius, Bacillus acidoterrestris, Bacillus agri, Bacillus aizawai, Bacillus albolactis, Bacillus alcalophilus, Bacillus alvei, Bacillus aminoglucosidicus, Bacillus aminovorans, Bacillus amylolyticus (also known as Paenibacillus amylolyticus) Bacillus amyloliquefaciens, Bacillus aneurinolyticus, Bacillus atrophaeus, Bacillus azotoformans, Bacillus badius, Bacillus cereus (synonyms: Bacillus endorhythmos, Bacillus medusa), Bacillus chitinosporus, Bacillus circulans, Bacillus coagulans, Bacillus endoparasiticus Bacillus fastidiosus, Bacillus firmus, Bacillus kurstaki, Bacillus lacticola, Bacillus lactimorbus, Bacillus lactis, Bacillus laterosporus (also known as Brevibacillus laterosporus), Bacillus lautus, Bacillus lentimorbus, Bacillus lentus, Bacillus licheniformis, Bacillus maroccanus, Bacillus megaterium, Bacillus metiens, Bacillus mycoides, Bacillus natto, Bacillus nematocida, Bacillus nigrificans, Bacillus nigrum, Bacillus pantothenticus, Bacillus popillae, Bacillus psychrosaccharolyticus, Bacillus pumilus, Bacillus siamensis, Bacillus smithii, Bacillus sphaericus, Bacillus subtilis, Bacillus thuringiensis, Bacillus uniflagellatus, Bradyrhizobium japonicum, Brevibacillus brevis Brevibacillus laterosporus (formerly Bacillus laterosporus), Chromobacterium subtsugae, Delftia acidovorans, Lactobacillus acidophilus, Lysobacter antibioticus, Lysobacter enzymogenes, Paenibacillus alvei, Paenibacillus polymyxa, Paenibacillus popilliae (formerly Bacillus popilliae), Pantoea agglomerans, Pasteuria penetrans (formerly Bacillus penetrans), Pasteuria usgae, Pectobacterium carotovorum (formerly Erwinia carotovora), Pseudomonas aeruginosa, Pseudomonas aureofaciens, Pseudomonas cepacia (formerly known as Burkholderia cepacia), Pseudomonas chlororaphis, Pseudomonas fluorescens, Pseudomonas proradix, Pseudomonas putida, Pseudomonas syringae, Serratia entomophila, Serratia marcescens, Streptomyces colombiensis, Streptomyces galbus, Streptomyces goshikiensis, Streptomyces griseoviridis, Streptomyces lavendulae, Streptomyces prasinus, Streptomyces saraceticus, Streptomyces venezuelae, Xanthomonas campestris, Xenorhabdus luminescens, Xenorhabdus nematophila, Rhodococcus globerulus AQ719 (NRRL Accession No. B-21663), Bacillus sp. AQ175 (ATCC Accession No. 55608), Bacillus sp. AQ 177 (ATCC Accession No. 55609), Bacillus sp. AQ178 (ATCC Accession No. 53522), and Streptomyces sp. strain NRRL Accession No. B-30145. In some cases the bacterium may be Azotobacter chroococcum, Methanosarcina barkeri, Klesiella pneumoniae, Azotobacter vinelandii, Rhodobacter spharoides, Rhodobacter capsulatus, Rhodobcter palustris, Rhodosporillum rubrum, Rhizobium leguminosarum or Rhizobium etli.

In some cases the bacterium may be a species of Clostridium, for example Clostridium pasteurianum, Clostridium beijerinckii, Clostridium perfringens, Clostridium tetani, Clostridium acetobutylicum.

In some cases, bacteria used with the methods and compositions of the present disclosure may be cyanobacteria. Examples of cyanobacterial genuses include Anabaena (for example Anagaena sp. PCC7120), Nostoc (for example Nostoc punctiforme), or Synechocystis (for example Synechocystis sp. PCC6803).

In some cases, bacteria used with the methods and compositions of the present disclosure may belong to the phylum Chlorobi, for example Chlorobium tepidum.

In some cases, microbes used with the methods and compositions of the present disclosure may comprise a gene homologous to a known NifH gene. Sequences of known NifH genes may be found in, for example, the Zehr lab NifH database, (wwwzehr.pmc.ucsc.edu/nifH_Database_Public/, Apr. 4, 2014), or the Buckley lab NifH database (www.css.cornell.edu/faculty/buckley/nifh.htm, and Gaby, John Christian, and Daniel H. Buckley. “A comprehensive aligned nifH gene database: a multipurpose tool for studies of nitrogen-fixing bacteria.” Database 2014 (2014): bau001.). In some cases, microbes used with the methods and compositions of the present disclosure may comprise a sequence which encodes a polypeptide with at least 60%, 70%, 80%, 85%, 90%, 95%, 96%, 96%, 98%, 99% or more than 99% sequence identity to a sequence from the Zehr lab NifH database, (wwwzehr.pmc.ucsc.edu/nifH_Database_Public/, Apr. 4, 2014). In some cases, microbes used with the methods and compositions of the present disclosure may comprise a sequence which encodes a polypeptide with at least 60%, 70%, 80%, 85%, 90%, 95%, 96%, 96%, 98%, 99% or more than 99% sequence identity to a sequence from the Buckley lab NifH database, (Gaby, John Christian, and Daniel H. Buckley. “A comprehensive aligned nifH gene database: a multipurpose tool for studies of nitrogen-fixing bacteria.” Database 2014 (2014): bau001.).

Microbes useful in the methods and compositions disclosed herein can be obtained by extracting microbes from surfaces or tissues of native plants; grinding seeds to isolate microbes; planting seeds in diverse soil samples and recovering microbes from tissues; or inoculating plants with exogenous microbes and determining which microbes appear in plant tissues. Non-limiting examples of plant tissues include a seed, seedling, leaf, cutting, plant, bulb, tuber, root, and rhizomes. In some cases, bacteria are isolated from a seed. The parameters for processing samples may be varied to isolate different types of associative microbes, such as rhizospheric, epiphytes, or endophytes. Bacteria may also be sourced from a repository, such as environmental strain collections, instead of initially isolating from a first plant. The microbes can be genotyped and phenotyped, via sequencing the genomes of isolated microbes; profiling the composition of communities in platita; characterizing the transcriptomic functionality of communities or isolated microbes; or screening microbial features using selective or phenotypic media (e.g., nitrogen fixation or phosphate solubilization phenotypes). Selected candidate strains or populations can be obtained via sequence data; phenotype data; plant data (e.g., genome, phenotype, and/or yield data); soil data (e.g., pH, N/P/K content, and/or bulk soil biotic communities); or any combination of these.

The bacteria and methods of producing bacteria described herein may apply to bacteria able to self-propagate efficiently on the leaf surface, root surface, or inside plant tissues without inducing a damaging plant defense reaction, or bacteria that are resistant to plant defense responses. The bacteria described herein may be isolated by culturing a plant tissue extract or leaf surface wash in a medium with no added nitrogen. However, the bacteria may be unculturable, that is, not known to be culturable or difficult to culture using standard methods known in the art. The bacteria described herein may be an endophyte or an epiphyte or a bacterium inhabiting the plant rhizosphere (rhizospheric bacteria). The bacteria obtained after repeating the steps of introducing genetic variation, exposure to a plurality of plants, and isolating bacteria from plants with an improved trait one or more times (e.g. 1, 2, 3, 4, 5, 10, 15, 25, or more times) may be endophytic, epiphytic, or rhizospheric. Endophytes are organisms that enter the interior of plants without causing disease symptoms or eliciting the formation of symbiotic structures, and are of agronomic interest because they can enhance plant growth and improve the nutrition of plants (e.g., through nitrogen fixation). The bacteria can be a seed-borne endophyte. Seed-borne endophytes include bacteria associated with or derived from the seed of a grass or plant, such as a seed-borne bacterial endophyte found in mature, dry, undamaged (e.g., no cracks, visible fungal infection, or prematurely germinated) seeds. The seed-borne bacterial endophyte can be associated with or derived from the surface of the seed; alternatively, or in addition, it can be associated with or derived from the interior seed compartment (e.g., of a surface-sterilized seed). In some cases, a seed-borne bacterial endophyte is capable of replicating within the plant tissue, for example, the interior of the seed. Also, in some cases, the seed-borne bacterial endophyte is capable of surviving desiccation.

The bacterial isolated according to methods of the disclosure, or used in methods or compositions of the disclosure, can comprise a plurality of different bacterial taxa in combination. By way of example, the bacteria may include Proteobacteria (such as Pseudomonas, Enterobacter, Stenotrophomonas, Burkholderia, Rhizobium, Herbaspirillum, Pantoea, Serratia, Rahnella, Azospirillum, Azorhizobium, Azotobacter, Duganella, Delftia, Bradyrhizobiun, Sinorhizobium and Halomonas), Firmicutes (such as Bacillus, Paenibacillus, Lactobacillus, Mycoplasma, and Acetabacterium), and Actinobacteria (such as Streptomyces, Rhoclacoccus, Microbacterium, and Curtobacterium). The bacteria used in methods and compositions of this disclosure may include nitrogen fixing bacterial consortia of two or more species. In some cases, one or more bacterial species of the bacterial consortia may be capable of fixing nitrogen. In some cases, one or more species of the bacterial consortia may facilitate or enhance the ability of other bacteria to fix nitrogen. The bacteria which fix nitrogen and the bacteria which enhance the ability of other bacteria to fix nitrogen may be the same or different. In some examples, a bacterial strain may be able to fix nitrogen when in combination with a different bacterial strain, or in a certain bacterial consortia, but may be unable to fix nitrogen in a monoculture. Examples of bacterial genuses which may be found in a nitrogen fixing bacterial consortia include, but are not limited to, Herbaspirillum, Azospirillum, Enterobacter, and Bacillus.

Bacteria that can be produced by the methods disclosed herein include Azotobacter sp., Bradyrhizobium sp., Klebsiella sp., and Sinorhizobium sp. In some cases, the bacteria may be selected from the group consisting of: Azotobacter vinelandii, Bradyrhizobium japonicum, Klebsiella pneumoniae, and Sinorhizobium meliloti. In some cases, the bacteria may be of the genus Enterobacter or Rahnella. In some cases, the bacteria may be of the genus Frankia, or Clostridium. Examples of bacteria of the genus Clostridium include, but are not limited to, Clostridium acetobutilicum, Clostridium pasteurianum, Clostridium beijerinckii, Clostridium perfringens, and Clostridium tetani. In some cases, the bacteria may be of the genus Paenibacillus, for example Paenibacillus azotofixans, Paenibacillus borealis, Paenibacillus durus, Paenibacillus macerans, Paenibacillus polymyxa, Paenibacillus alvei, Paenibacillus amylolyticus, Paenibacillus campinasensis, Paenibacillus chibensis, Paenibacillus glucanolyvticus, Paenibacillus illinoisensis, Paenibacillus larvae subsp. Larvae, Paenibacillus larvae subsp. Pulvifaciens, Paenibacillus lautus, Paenibacillus macerans, Paenibacillus macquariensis, Paenibacillus macquariensis, Paenibacillus pabuli, Paenibacillus peoriae, or Paenibacillus polymyxa.

In some examples, bacteria isolated according to methods of the disclosure can be a member of one or more of the following taxa: Achromobacter, Acidithiobacillus, Acidovorax, Acidovoraz, Acinetobacter, Actinoplanes, Adlercreutzia, Aerococcus, Aeromonas, Afipia, Agromyces, Ancylobacter, Arthrobacter, Atopostipes, Azospirillum, Bacillus, Bdellovibrio, Beijerinckia, Bosea, Bradyrhizobium, Brevibacillus, Brevundimonas, Burkholderia, Candidatus Haloredivivus, Caulobacter, Cellulomonas, Cellvibrio, Chryseobacterium, Citrobacter, Clostridium, Coraliomargarita, Corynebacterium, Cupriavidus, Curtobacterium, Curvibacter, Deinococcus, Delftia, Desemzia, Devosia, Dokdonella, Dyella, Enhydrobacter, Enterobacter, Enterococcus, Erwinia, Escherichia, Escherichia/Shigella, Exiguobacterium, Ferroglobus, Filimonas, Finegoldia, Flavisolibacter, Flavobacterium, Frigoribacterium, Gluconacetobacter, Hafnia, Halobaculum, Halomonas, Halosimplex, Herbaspirillum, Hymenobacter, Klebsiella, Kocuria, Kosakonia, Lactobacillus, Leclercia, Lentzea, Luteibacter, Luteimonas, Massilia, Mesorhizobium, Methylobacterium, Microbacterium, Micrococcus, Microvirga, Mycobacterium, Neisseria, Nocardia, Oceanibaculum, Ochrobactrum, Okibacterium, Oligotropha, Oryzihumus, Oxalophagus, Paenibacillus, Panteoa, Pantoea, Pelomonas, Perlucidibaca, Plantibacter, Polynucleobacter, Propionibacterium, Propioniciclava, Pseudoclavibacter, Pseudomonas, Pseudonocardia, Pseudoxanthomonas, Psychrobacter, Rahnella, Ralstonia, Rheinheimera, Rhizobium, Rhodococcus, Rhodopseudomonas, Roseateles, Ruminococcus, Sebaldella, Sediminibacillus, Sediminibacterium, Serratia, Shigella, Shinella, Sinorhizobium, Sinosporangium, Sphingobacterium, Sphingomonas, Sphingopyxis, Sphingosinicella, Staphylococcus, 25 Stenotrophomonas, Strenotrophomonas, Streptococcus, Streptomyces, Stygiolobus, Sulfurisphaera, Tatumella, Tepidimonas, Thermomonas, Thiobacillus, Variovorax, WPS-2 genera incertae sedis, Xanthomonas, and Zimmermannella.

In some cases, a bacterial species selected from at least one of the following genera are utilized: Enterobacter, Klebsiella, Kosakonia, and Rahnella. In some cases, a combination of bacterial species from the following genera are utilized: Enterobacter, Klebsiella, Kosakonia, and Rahnella. In some cases, the species utilized can be one or more of: Enterobacter sacchari, Klebsiella variicola, Kosakonia sacchari, and Rahnella aquatilis.

In some cases, a Gram positive microbe may have a Molybdenum-Iron nitrogenase system comprising: nifH, nifD, nifK, nifB, nifE, nifN, nifX, hesA, nifV nifW, nifU, nifS, nifII, and nifI2. In some cases, a Gram positive microbe may have a vanadium nitrogenase system comprising: vafDG, vnƒK, vnƒE, vnƒN, vupC, vupB, vupA, vnƒV, vnƒR1, vnƒH, vnƒR2, vnƒA (transcriptional regulator). In some cases, a Gram positive microbe may have an iron-only nitrogenase system comprising: anƒK, anƒG, anƒD, anƒH, anƒA (transcriptional regulator). In some cases, a Gram positive microbe may have a nitrogenase system comprising glnB, and glnK (nitrogen signaling proteins). Some examples of enzymes involved in nitrogen metabolism in Gram positive microbes include glnA (glutamine synthetase), gdh (glutamate dehydrogenase), bdh (3-hydroxybutyrate dehydrogenase), glutaminase, gltAB/gltB/gltS (glutamate synthase), asnA/asnB (aspartate- ammonia ligase/asparagine synthetase), and ansA/ansZ (asparaginase). Some examples of proteins involved in nitrogen transport in Gram positive microbes include amtB (ammonium transporter), glnK (regulator of ammonium transport), glnPHQ/ glnQHMP (ATP-dependent glutamine/glutamate transporters), glnT/alsT/yrbD/yƒlA (glutamine-like proton symport transporters), and gltP/gltT/yhcl/ngt (glutamate-like proton symport transporters).

Examples of Gram positive microbes which may be of particular interest include Paenibacillus polymixa, Paenibacillus riograndensis, Paenibacillus sp., Frankia sp., Heliobacterium sp., Heliobacterium chlorum, Heliobacillus sp., Heliophilum sp., Heliorestis sp., Clostridium acetobutylicum, Clostridium sp., Mycobacterium flaum, Mycobacterium sp., Arthrobacter sp., Agromyces sp., Corynebacterium autitrophicum, Corynebacterium sp., Micromonspora sp., Propionibacteria sp., Streptomyces sp., and Microbacterium sp..

Some examples of genetic alterations which may be made in Gram positive microbes include: deleting glnR to remove negative regulation of BNF in the presence of environmental nitrogen, inserting different promoters directly upstream of the nif cluster to eliminate regulation by GlnR in response to environmental nitrogen, mutating glnA to reduce the rate of ammonium assimilation by the GS-GOGAT pathway, deleting amtB to reduce uptake of ammonium from the media, mutating glnA so it is constitutively in the feedback-inhibited (FBI-GS) state, to reduce ammonium assimilation by the GS-GOGAT pathway.

In some cases, glnR is the main regulator of N metabolism and fixation in Paenibacillus species. In some cases, the genome of a Paenibacillus species may not contain a gene to produce glnR. In some cases, the genome of a Paenibacillus species may not contain a gene to produce glnE or glnD. In some cases, the genome of a Pαenibacillus species may contain a gene to produce glnB or glnK. For example, Pαenibacillus sp. WLY78 doesn’t contain a gene for glnB, or its homologs found in the archaeon Methanococcus maripaludis, nifl1 and nifl2. In some cases, the genomes of Paenibacillus species may be variable. For example, Paenibacillus polymixa E681 lacks glnK and gdh, has several nitrogen compound transporters, but only amtB appears to be controlled by GlnR. In another example, Paenibacillus sp. JDR2 has glnK, gdh and most other central nitrogen metabolism genes, has many fewer nitrogen compound transporters, but does have glnP HQ controlled by GlnR. Paenibacillus riograndensis SBR5 contains a standard glnRA operon, an ƒdx gene, a main niƒ operon, a secondary niƒ operon, and an anƒ operon (encoding iron-only nitrogenase). Putative glnR/tnrA sites were found upstream of each of these operons. GlnR may regulate all of the above operons, except the anƒ operon. GlnR may bind to each of these regulatory sequences as a dimer.

Paenibacillus N-fixing strains may fall into two subgroups: Subgroup I, which contains only a minimal nif gene cluster and subgroup II, which contains a minimal cluster, plus an uncharacterized gene between nifX and hesA, and often other clusters duplicating some of the nif genes, such as nifH, nifHDK, nifBEN, or clusters encoding vanadaium nitrogenase (vnf) or iron-only nitrogenase (anƒ) genes.

In some cases, the genome of a Paenibacillus species may not contain a gene to produce glnB or glnK. In some cases, the genome of a Paenibacillus species may contain a minimal nif cluster with 9 genes transcribed from a sigma-70 promoter. In some cases, a Paenibacillus nif cluster may be negatively regulated by nitrogen or oxygen. In some cases, the genome of a Paenibacillus species may not contain a gene to produce sigma-54. For example, Paenibacillus sp. WLY78 does not contain a gene for sigma-54. In some cases, a nif cluster may be regulated by glnR, and/or TnrA. In some cases, activity of a nif cluster may be altered by altering activity of glnR, and/or TnrA.

In Bacilli, glutamine synthetase (GS) is feedback-inhibited by high concentrations of intracellular glutamine, causing a shift in confirmation (referred to as FBI-GS). Nif clusters contain distinct binding sites for the regulators GlnR and TnrA in several Bacilli species. GlnR binds and represses gene expression in the presence of excess intracellular glutamine and AMP. A role of GlnR may be to prevent the influx and intracellular production of glutamine and ammonium under conditions of high nitrogen availability. TnrA may bind and/or activate (or repress) gene expression in the presence of limiting intracellular glutamine, and/or in the presence of FBI-GS. In some cases, the activity of a Bacilli nif cluster may be altered by altering the activity of GlnR.

Feedback-inhibited glutamine synthetase (FBI-GS) may bind GlnR and stabilize binding of GlnR to recognition sequences. Several bacterial species have a GlnR/TnrA binding site upstream of the nif cluster. Altering the binding of FBI-GS and GlnR may alter the activity of the nif pathway.

### Budapest Treaty on the International Recognition of the Deposit of Microorganisms for the Purpose of Patent Procedures

The microbial deposits of the present disclosure were made under the provisions of the Budapest Treaty on the International Recognition of the Deposit of Microorganisms for the Purpose of Patent Procedure (Budapest Treaty).

Applicants state that pursuant to 37 C.F.R. § 1.808(a)(2) “all restrictions imposed by the depositor on the availability to the public of the deposited material will be irrevocably removed upon the granting of the patent.” This statement is subject to paragraph (b) of this section (i.e. 37 C.F.R. § 1.808(b)).

The Enterobacter sacchari has now been reclassified as Kosakonia sacchari, the name for the organism may be used interchangeably throughout the manuscript.

Many microbes of the present disclosure are derived from two wild-type strains. Strain CI006 is a bacterial species previously classified in the genus Enterobacter (see aforementioned reclassification into Kosakonia). Strain CI019 is a bacterial species classified in the genus Rahnella. The deposit information for the CI006 Kosakonia wild type (WT) and CI019 Rahnella WT are found in the below Table 1.

Some microorganisms described in this application were deposited on Jan. 06, 2017 or Aug. 11, 2017 with the Bigelow National Center for Marine Algae and Microbiota (NCMA), located at 60 Bigelow Drive, East Boothbay, Maine 04544, USA. As aforementioned, all deposits were made under the terms of the Budapest Treaty on the International Recognition of the Deposit of Microorganisms for the Purposes of Patent Procedure. The Bigelow National Center for Marine Algae and Microbiota accession numbers and dates of deposit for the aforementioned Budapest Treaty deposits are provided in Table 1.

Biologically pure cultures of Kosakonia sacchari (WT), Rahnella aquatilis (WT), and a variant/remodeled Kosakonia sacchari strain were deposited on Jan. 06, 2017 with the Bigelow National Center for Marine Algae and Microbiota (NCMA), located at 60 Bigelow Drive, East Boothbay, Maine 04544, USA, and assigned NCMA Patent Deposit Designation numbers 201701001, 201701003, and 201701002, respectively. The applicable deposit information is found below in Table 1.

Biologically pure cultures of variant/remodeled Kosakonia sacchari strains were deposited on Aug. 11, 2017 with the Bigelow National Center for Marine Algae and Microbiota (NCMA), located at 60 Bigelow Drive, East Boothbay, Maine 04544, USA, and assigned NCMA Patent Deposit Designation numbers 201708004, 201708003, and 201708002, respectively. The applicable deposit information is found below in Table 1.

A biologically pure culture of Klebsiella variicola (WT) was deposited on Aug. 11, 2017 with the Bigelow National Center for Marine Algae and Microbiota (NCMA), located at 60 Bigelow Drive, East Boothbay, Maine 04544, USA, and assigned NCMA Patent Deposit Designation number 201708001. Biologically pure cultures of two Klebsiella variicola variants/remodeled strains were deposited on Dec. 20, 2017 with the Bigelow National Center for Marine Algae and Microbiota (NCMA), located at 60 Bigelow Drive, East Boothbay, Maine 04544, USA, and assigned NCMA Patent Deposit Designation numbers 201712001 and 201712002, respectively. The applicable deposit information is found below in Table 1.

Biologically pure cultures of two Kosakonia sacchari variants/remodeled strains were deposited on Dec. 23, 2019 with the American Type Culture Collection (ATCC), located at 10801 University Boulevard, Manassas, Virginia 20110-2209, USA and assigned ATCC Patent Deposit Numbers PTA-126575 and PTA-126576. Biologically pure cultures of four Klebsiella variicola variants/remodeled strains were deposited on Dec. 23, 2019 with the American Type Culture Collection (ATCC), located at 10801 University Boulevard, Manassas, Virginia 20110-2209, USA and assigned ATCC Patent Deposit Numbers PTA-126577, PTA-126578, PTA-126579 and PTA-126580. A biologically pure culture of a Paenibacillus polymyxa (WT) strain was deposited on Dec. 23, 2019 with the American Type Culture Collection (ATCC), located at 10801 University Boulevard, Manassas, Virginia 20110-2209, USA and assigned ATCC Patent Deposit Number PTA-126581. A biologically pure culture of a Paraburkholderia tropica (WT) strain was deposited on Dec. 23, 2019 with the American Type Culture Collection (ATCC), located at 10801 University Boulevard, Manassas, Virginia 20110-2209, USA and assigned ATCC Patent Deposit Number PTA-126582. A biologically pure culture of a Herbaspirillum aquaticum (WT) strain was deposited on Dec. 23, 2019 with the American Type Culture Collection (ATCC), located at 10801 University Boulevard, Manassas, Virginia 20110-2209, USA and assigned ATCC Patent Deposit Number PTA-126583. Biologically pure cultures of four Metakosakonia intestini variants/remodeled strains were deposited on Dec. 23, 2019 with the American Type Culture Collection (ATCC), located at 10801 University Boulevard, Manassas, Virginia 20110-2209, USA and assigned ATCC Patent Deposit Numbers PTA-126584, PTA-126586, PTA-126587 and PTA-126588. A biologically pure culture of a Metakosakonia intestini (WT) strain was deposited on Dec. 23, 2019 with the American Type Culture Collection (ATCC), located at 10801 University Boulevard, Manassas, Virginia 20110-2209, USA and assigned ATCC Patent Deposit Number PTA-126585. The applicable deposit information is found below in Table 1.

TABLE 1

Microorganisms Deposited under the Budapest Treaty

| Depository | Pivot Strain Designation (some strains have multiple designations) | Taxonomy | Accession Number | Date of Deposit |
|---|---|---|---|---|
| NCMA | CI006, PBC6.1, 6 | Kosakonia sacchari (WT) | 201701001 | Jan. 06, 2017 |
| NCMA | CI019, 19 | Rahnella aquatilis (WT) | 201701003 | Jan. 06, 2017 |
| NCMA | CM029, 6-412 | Kosakonia sacchari | 201701002 | Jan. 06, 2017 |
| NCMA | 6-403 CM037 | Kosakonia sacchari | 201708004 | Aug. 11, 2017 |
| NCMA | 6-404, CM38, PBC6.38 | Kosakonia sacchari | 201708003 | Aug. 11, 2017 |
| NCMA | CM094, 6-881, PBC6.94 | Kosakonia sacchari | 201708002 | Aug. 11, 2017 |
| NCMA | CI137, 137, PB137 | Klebsiella variicola (WT) | 201708001 | Aug. 11, 2017 |
| NCMA | 137-1034 | Klebsiella variicola | 201712001 | Dec. 20, 2017 |
| NCMA | 137-1036 | Klebsiella variicola | 201712002 | Dec. 20, 2017 |
| ATCC | 6-2425 | Kosakonia sacchari | PTA-126575 | Dec. 23, 2019 |
| ATCC | 06-2634 | Kosakonia sacchari | PTA-126576 | Dec. 23, 2019 |
| ATCC | 137-1968 | Klebsiella variicola | PTA-126577 | Dec. 23, 2019 |
| ATCC | 137-2219 | Klebsiella variicola | PTA-126578 | Dec. 23, 2019 |
| ATCC | 137-2237 | Klebsiella variicola | PTA-126579 | Dec. 23, 2019 |
| ATCC | 137-2285 | Klebsiella variicola | PTA-126580 | Dec. 23, 2019 |
| ATCC | 41 | Paenibacillus polymyxa (WT) | PTA-126581 | Dec. 23, 2019 |
| ATCC | 8 | Paraburkholderia tropica (WT) | PTA-126582 | Dec. 23, 2019 |
| ATCC | 3069 | Herbaspirillum aquaticum (WT) | PTA-126583 | Dec. 23, 2019 |
| ATCC | 910-3655 | Metakosakonia intestini | PTA-126584 | Dec. 23, 2019 |
| ATCC | 910 | Metakosakonia intestini (WT) | PTA-126585 | Dec. 23, 2019 |
| ATCC | 910-3963 | Metakosakonia intestini | PTA-126586 | Dec. 23, 2019 |
| ATCC | 910-3961 | Metakosakonia intestini | PTA-126587 | Dec. 23, 2019 |
| ATCC | 910-3994 | Metakosakonia intestini | PTA-126588 | Dec. 23, 2019 |

### Isolated and Biologically Pure Microorganisms

The present disclosure, in certain embodiments, provides isolated and biologically pure microorganisms that have applications, inter alia, in agriculture. The disclosed microorganisms can be utilized in their isolated and biologically pure states, as well as being formulated into compositions (see below section for exemplary composition descriptions). Furthermore, the disclosure provides microbial compositions containing at least two members of the disclosed isolated and biologically pure microorganisms, as well as methods of utilizing said microbial compositions. Furthermore, the disclosure provides for methods of modulating nitrogen fixation in plants via the utilization of the disclosed isolated and biologically pure microbes.

### Agricultural Compositions

In some embodiments, whole plant nitrogen heterogeneity in a field can be reduced using an agricultural composition. Agricultural compositions comprising bacteria or bacterial populations produced according to methods described herein and/or having characteristics as described herein can be in the form of a liquid, a foam, or a dry product. Compositions comprising bacteria or bacterial populations produced according to methods described herein and/or having characteristics as described herein may also be used to improve plant traits. In some examples, a composition comprising bacterial populations may be in the form of a dry powder, a slurry of powder and water, or a flowable seed treatment. The compositions comprising bacterial populations may be coated on a surface of a seed, and may be in liquid form.

The composition can be fabricated in bioreactors such as continuous stirred tank reactors, batch reactors, and on the farm. In some examples, compositions can be stored in a container, such as a jug or in mini bulk. In some examples, compositions may be stored within an object selected from the group consisting of a bottle, jar, ampule, package, vessel, bag, box, bin, envelope, carton, container, silo, shipping container, truck bed, and case.

Bacterial compositions described herein can be formulated using an agriculturally acceptable carrier. The formulation useful for these embodiments may include at least one member selected from the group consisting of a tackifier, a microbial stabilizer, a fungicide, an antibacterial agent, a preservative, a stabilizer, a surfactant, an anti-complex agent, an herbicide, a nematicide, an insecticide, a plant growth regulator, a fertilizer, a rodenticide, a dessicant, a bactericide, a nutrient, and any combination thereof. In some examples, compositions may be shelf-stable. For example, any of the compositions described herein can include an agriculturally acceptable carrier (e.g., one or more of a fertilizer such as a non-naturally occurring fertilizer, an adhesion agent such as a non- naturally occurring adhesion agent, and a pesticide such as a non-naturally occurring pesticide). A non-naturally occurring adhesion agent can be, for example, a polymer, copolymer, or synthetic wax. For example, any of the coated seeds, seedlings, or plants described herein can contain such an agriculturally acceptable carrier in the seed coating. In any of the compositions or methods described herein, an agriculturally acceptable carrier can be or can include a non-naturally occurring compound (e.g., a non-naturally occurring fertilizer, a non-naturally occurring adhesion agent such as a polymer, copolymer, or synthetic wax, or a non-naturally occurring pesticide).

### Fertilizers, Nitrogen Stabilizers, and Urease Inhibitors

In some embodiments, fertilizers, nitrogen stabilizers and/or urease inhibitors are used in combination with the methods and bacteria of the present discosure. Urease inhibitors are chemical compounds that block the activity of the enzyme urease, for example by inhibiting hydrolytic action on urea by the enzyme urease. Example urease inhibitors include, but are not limited to, N-(n-butyl)-thiophosphoric triamide (NBPT), AGROTAIN, AGROTAIN PLUS, and AGROTAIN PLUS SC. Further, the disclosure contemplates utilization of AGROTAIN ADVANCED 1.0, AGROTAIN DRI-MAXX, and AGROTAIN ULTRA.

Thousands of chemicals have been evaluated as soil urease inhibitors (Kiss and Simihaian, 2002). However, only a few of the many compounds tested meet the necessary requirements of being non toxic, effective at low concentration, stable, and compatible with urea (solid and solutions), degradable in the soil and inexpensive. They can be classified according to their structures and their assumed interaction with the enzyme urease (Watson, 2000, 2005). Four main classes of urease inhibitors have been proposed: (a) reagents which interact with the sulphydryl groups (sulphydryl reagents), (b) hydroxamates, (c) agricultural crop protection chemicals, and (d) structural analogues of urea and related compounds. N-(n-Butyl) thiophosphoric triamide (NBPT), phenylphosphorodiamidate (PPD/ PPDA), and hydroquinone are probably the most thoroughly studied urease inhibitors (Kiss and Simihaian, 2002). Research and practical testing has also been carried out with N-(2-nitrophenyl) phosphoric acid triamide (2-NPT) and ammonium thiosulphate (ATS). The organo-phosphorus compounds are structural analogues of urea and are some of the most effective inhibitors of urease activity, blocking the active site of the enzyme (Watson, 2005).

### Concentrations and Rates of Application of Agricultural Compositions

As aforementioned, the agricultural compositions of the present disclosure, which comprise a taught microbe, can be applied to plants in a multitude of ways. In two particular aspects, the disclosure contemplates an in-furrow treatment or a seed treatment

For seed treatment embodiments, the microbes of the disclosure can be present on the seed in a variety of concentrations. For example, the microbes can be found in a seed treatment at a cfu concentration, per seed of: 1 × 10¹, 1 × 10², 1 × 10³, 1 × 10⁴, 1 × 10⁵, 1 × 10⁶, 1 × 10⁷, 1 × 10⁸, 1 × 10⁹, 1 × 10¹⁰, or more. In particular aspects, the seed treatment compositions comprise about 1 × 10⁴ to about 1 × 10⁸ cfu per seed. In other particular aspects, the seed treatment compositions comprise about 1 × 10⁵ to about 1 × 10⁷ cfu per seed. In other aspects, the seed treatment compositions comprise about 1 × 10⁶ cfu per seed.

In the United States, about 10% of corn acreage is planted at a seed density of above about 36,000 seeds per acre; ⅓ of the corn acreage is planted at a seed density of between about 33,000 to 36,000 seeds per acre; ⅓ of the corn acreage is planted at a seed density of between about 30,000 to 33,000 seeds per acre, and the remainder of the acreage is variable. See, “Corn Seeding Rate Considerations,” written by Steve Butzen, available at: www.pioneer.com/home/site/us/agronomy/library/corn-seeding-rate-considerations/

Table 2 below utilizes various cfu concentrations per seed in a contemplated seed treatment embodiment (rows across) and various seed acreage planting densities (1^{st} column: 15K-41K) to calculate the total amount of cfu per acre, which would be utilized in various agricultural scenarios (i.e. seed treatment concentration per seed × seed density planted per acre). Thus, if one were to utilize a seed treatment with 1 × 10⁶ cfu per seed and plant 30,000 seeds per acre, then the total cfu content per acre would be 3 × 10¹⁰ (i.e. 30 K * 1 × 10⁶).

TABLE 2

Total CFU Per Acre Calculation for Seed Treatment Embodiments

| Corn Population (i.e. seeds per acre) | 1.00E-+02 | 1.00E+03 | 1.00E+04 | 1.00E+05 | 1.00E+06 | 1.00E+07 | 1.00E+08 | 1.00E+09 |
|---|---|---|---|---|---|---|---|---|
| 15,000 | 1.50E+06 | 1.50E+07 | 1.50E+08 | 1.50E+09 | 1.50E+10 | 1.50E+11 | 1.50E+12 | 1.50E+13 |
| 16,000 | 1.60E+06 | 1.60E+07 | 1.60E+08 | 1.60E+09 | 1.60E+10 | 1.60E+11 | 1.60E+12 | 1.60E+13 |
| 17,000 | 1.70E+06 | 1.70E+07 | 1.70E+08 | 1.70E+09 | 1.70E+10 | 1.70E+11 | 1.70E+12 | 1.70E+13 |
| 18,000 | 1.80E+06 | 1.80E+07 | 1.80E+08 | 1.80E+09 | 1.80E+10 | 1.80E+11 | 1.80E+12 | 1.80E+13 |
| 19,000 | 1.90E+06 | 1.90E+07 | 1.90E+08 | 1.90E+09 | 1.90E+10 | 1.90E+11 | 1.90E+12 | 1.90E+13 |
| 20,000 | 2.00E+06 | 2.00E+07 | 2.00E+08 | 2.00E+09 | 2.00E+10 | 2.00E+11 | 2.00E+12 | 2.00E+13 |
| 21,000 | 2.10E+06 | 2.10E+07 | 2.10E+08 | 2.10E+09 | 2.10E+10 | 2.10E+11 | 2.10E+12 | 2.10E+13 |
| 22,000 | 2.20E+06 | 2.20E+07 | 2.20E+08 | 2.20E+09 | 2.20E+10 | 2.20E+11 | 2.20E+12 | 2.20E+13 |
| 23,000 | 2.30E+06 | 2.30E+07 | 2.30E+08 | 2.30E+09 | 2.30E+10 | 2.30E+11 | 2.30E+12 | 2.30E+13 |
| 24,000 | 2.40E+06 | 2.40E+07 | 2.40E+08 | 2.40E+09 | 2.40E+10 | 2.40E+11 | 2.40E+12 | 2.40E+13 |
| 25,000 | 2.50E+06 | 2.50E+07 | 2.50E+08 | 2.50E+09 | 2.50E+10 | 2.50E+11 | 2.50E+12 | 2.50E+13 |
| 26,000 | 2.60E+06 | 2.60E+07 | 2.60E+08 | 2.60E+09 | 2.60E+10 | 2.60E+11 | 2.60E+12 | 2.60E+13 |
| 27,000 | 2.70E+06 | 2.70E+07 | 2.70E+08 | 2.70E+09 | 2.70E+10 | 2.70E+11 | 2.70E+12 | 2.70E+13 |
| 28,000 | 2.80E+06 | 2.80E+07 | 2.80E+08 | 2.80E+09 | 2.80E+10 | 2.80E+11 | 2.80E+12 | 2.80E+13 |
| 29,000 | 2.90E+06 | 2.90E+07 | 2.90E+08 | 2.90E+09 | 2.90E+10 | 2.90E+11 | 2.90E+12 | 2.90E+13 |
| 30,000 | 3.00E+06 | 3.00E+07 | 3.00E+08 | 3.00E+09 | 3.00E+10 | 3.00E+11 | 3.00E+12 | 3.00E+13 |
| 31,000 | 3.10E+06 | 3.10E+07 | 3.10E+08 | 3.10E+09 | 3.10E+10 | 3.10E+11 | 3.10E+12 | 3.10E+13 |
| 32,000 | 3.20E+06 | 3.20E+07 | 3.20E+08 | 3.20E+09 | 3.20E+10 | 3.20E+11 | 3.20E+12 | 3.20E+13 |
| 33,000 | 3.30E+06 | 3.30E+07 | 3.30E+08 | 3.30E+09 | 3.30E+10 | 3.30E+11 | 3.30E+12 | 3.30E+13 |
| 34,000 | 3.40E+06 | 3.40E+07 | 3.40E+08 | 3.40E+09 | 3.40E+10 | 3.40E+11 | 3.40E+12 | 3.40E+13 |
| 35,000 | 3.50E+06 | 3.50E+07 | 3.50E+08 | 3.50E+09 | 3.50E+10 | 3.50E+11 | 3.50E+12. | 3.50E+13 |
| 36,000 | 3.60E+06 | 3.60E+07 | 3.60E+08 | 3.60E+09 | 3.60E+10 | 3.60E+11 | 3.60E+12 | 3.60E+13 |
| 37,000 | 3.70E+06 | 3.70E+07 | 3.70E+08 | 3.70E+09 | 3.70E-10 | 3.70E+11 | 3.70E+12 | 3.70E+13 |
| 38,000 | 3.80E+06 | 3.80E+07 | 3.80E+08 | 3.80E+09 | 3.80E+10 | 3.80E+11 | 3.80E+12 | 3.80E+13 |
| 39,000 | 3.90E+06 | 3.90E+07 | 3.90E+08 | 3.90E+09 | 3.90E+10 | 3.90E+11 | 3.90E+12 | 3.90E+13 |
| 40,000 | 4.00E+06 | 4.00E+07 | 4.00E+08 | 4.00E+09 | 4.00E+10 | 4.00E+11 | 4.00E+12 | 4.00E+13 |
| 41,000 | 4.10E+06 | 4.10E1+07 | 4.10E+08 | 4.10E+09 | 4.10E+10 | 4.10E+11 | 4.10E+12 | 4.10E+13 |

For in-furrow embodiments, the microbes of the disclosure can be applied at a cfu concentration per acre of: 1 × 10⁶, 3.20 × 10¹⁰, 1.60 × 10¹¹, 3.20 × 10¹¹, 8.0 × 10¹¹, 1.6 × 10¹², 3.20 × 10¹², or more. Therefore, in aspects, the liquid in-furrow compositions can be applied at a concentration of between about 1 × 10⁶ to about 3 × 10¹² cfu per acre.

In some aspects, the in-furrow compositions are contained in a liquid formulation. In the liquid in-furrow embodiments, the microbes can be present at a cfu concentration per milliliter of: 1 × 10¹, 1 × 10², 1 × 10³, 1 × 10⁴, 1 × 10⁵, 1 × 10⁶, 1 × 10⁷, 1 × 10⁸, 1 × 10⁹, 1 × 10¹⁰, 1 × 10¹¹, 1 × 10¹², 1 × 10¹³, or more. In certain aspects, the liquid in-furrow compositions comprise microbes at a concentration of about 1 × 10⁶ to about 1 × 10¹¹ cfu per milliliter. In other aspects, the liquid in-furrow compositions comprise microbes at a concentration of about 1 × 10⁷ to about 1 × 10¹⁰ cfu per milliliter. In other aspects, the liquid in-furrow compositions comprise microbes at a concentration of about 1 × 10⁸ to about 1 × 10⁹ cfu per milliliter. In other aspects, the liquid in-furrow compositions comprise microbes at a concentration of up to about 1 × 10¹³ cfu per milliliter.

TABLE 3

Table of Strains

| | | | | | |
|---|---|---|---|---|---|
| Name | Lineage | Mutagenic DNA Description | Genotype | Gene 1 mutation | Gene 2 mutation |
| CI006 | Isolated strain from Enterobacter (now Kosakonia) genera | None | WT | | |
| CI008 | Isolated strain from Burkholderia genera | None | WT | | |
| CI010 | Isolated strain from Klebsiella genera | None | WT | | |
| CI019 | Isolated strain from Rahnella genera | None | WT | | |
| CI028 | Isolated strain from Enterobacter genera | None | WT | | |
| CI050 | Isolated strain from Klebsiella genera | None | WT | | |
| CM002 | Mutant of CI050 | Disruption of nifL gene with a kanamycin resistance expression | ΔnitL::KanR | SEQ ID NO: 33 | |
| | | cassette (KanR) encoding the aminoglycoside O-phosphotransferase gene aph1 inserted. | | | |
| CM011 | Mutant of CI019 | Disruption of nifL gene with a spectinomycin resistance expression cassette (SpecR) encoding the streptomycin 3″-O-adenylyltransferase gene aadA inserted. | ΔnifL::SpecR | SEQ ID NO: 34 | |
| CM013 | Mutant of CI006 | Disruption of nifL gene with a kanamycin resistance expression cassette (KanR) encoding the aminoglycoside O-phosphotransferase gene aph1 inserted. | ΔnifL::KanR | SEQ ID NO: 35 | |
| CM004 | Mutant of CI010 | Disruption of amtB gene with a kanamycin resistance expression cassette (KanR) encoding the aminoglycoside O-phosphotransferase gene aph1 inserted. | ΔamtB::KanR | SEQ ID NO: 36 | |
| CM005 | Mutant of CI010 | Disruption of nifL gene with a kanamycin resistance expression cassette (KanR) encoding the aminoglycoside O-phosphotransferase gene aph1 inserted. | ΔnifL::KanR | SEQ ID NO: 37 | |
| CM015 | Mutant of CI006 | Disruption of nifL gene with a fragment of the region upstream of the ompX gene inserted (Prm5). | ΔnifL::Prm5 | SEQ ID NO: 38 | |
| CM021 | Mutant of CI006 | Disruption of nifL gene with a fragment of the region upstream of an unanotated gene and the first 73bp of that gene inserted (Prm2). | ΔnifL::Prm2 | SEQ ID NO: 39 | |
| CM023 | Mutant of CI006 | Disruption of nifL gene with a fragment of the region upstream of the | ΔnifL::Prm4 | SEQ ID NO: 40 | |
| | | acpP gene and the first 121bp of the acpP gene inserted (Prm4). | | | |
| CM014 | Mutant of CI006 | Disruption of nifL gene with a fragment of the region upstream of the 1pp gene and the first 29bp of the 1pp gene inserted (Prm1). | ΔnifL::Prm1 | SEQ ID NO: 41 | |
| CM016 | Mutant of CI006 | Disruption of nifL gene with a fragment of the region upstream of the lexA 3 gene and the first 21bp of the lexA 3 gene inserted (Prm9). | ΔnifL::Prm9 | SEQ ID NO: 42 | |
| CM022 | Mutant of CI006 | Disruption of nifL gene with a fragment of the region upstream of the mntP 1 gene and the first 53bp of the mntP 1 gene inserted (Prm3). | ΔnifL::Prm3 | SEQ ID NO: 43 | |
| CM024 | Mutant of CI006 | Disruption of nifL gene with a fragment of the region upstream of the sspA gene inserted (Prm7). | ΔnifL::Prm7 | SEQ ID NO: 44 | |
| CM025 | Mutant of CI006 | Disruption of nifL gene with a fragment of the region upstream of the hisS gene and the first 52bp of the hisS gene inserted (Prm10). | ΔnifL::Prm10 | SEQ ID NO: 45 | |
| CM006 | Mutant of CI010 | Disruption of glnB gene with a kanamycin resistance expression cassette (KanR) encoding the aminoglycoside O-phosphotransferase gene aph1 inserted. | ΔglnB::KanR | SEQ ID NO: 46 | |
| CM017 | Mutant of CI028 | Disruption of nifL gene with a kanamycin resistance expression cassette (KanR) encoding the aminoglycoside O-phosphotransferase gene aph1 inserted. | ΔnifL::KanR | SEQ ID NO: 47 | |
| CM011 | Mutant of CI019 | Disruption of nifL gene with a spectinomycin resistance expression | ΔnifL::SpecR | SEQ ID NO: 48 | |
| | | cassette (SpecR) encoding the streptomycin 3″-O-adenylyltransferase gene aadA inserted. | | | |
| CM013 | Mutant of CI006 | Disruption of nifL gene with a kanamycin resistance expression cassette (KanR) encoding the aminoglycoside O-phosphotransferase gene aph1 inserted. | ΔnifL::KanR | SEQ ID NO: 49 | |
| CM005 | Mutant of CI010 | Disruption of nifL gene with a kanamycin resistance expression cassette (KanR) encoding the aminoglycoside O-phosphotransferase gene aph1 inserted. | ΔnifL::KanR | SEQ ID NO: 50 | |
| CM014 | Mutant of CI006 | Disruption of nifL gene with a fragment of the region upstream of the 1pp gene and the first 29bp of the 1pp gene inserted (Prm1). | ΔnifL::Prm1 | SEQ ID NO: 51 | |
| CM015 | Mutant of CI006 | Disruption of nifL gene with a fragment of the region upstream of the ompX gene inserted (Prm5). | ΔnifL::Prm5 | SEQ ID NO: 52 | |
| CM023 | Mutant of CI006 | Disruption of nifL gene with a fragment of the region upstream of the acpP gene and the first 121bp of the acpP gene inserted (Prm4). | ΔnifL::Prm4 | SEQ ID NO: 53 | |
| CM029 | Mutant of CI006 | Disruption of nifL gene with a fragment of the region upstream of the ompX gene inserted (Prm5) and deletion of the 1287bp after the start codon of the glnE gene containing the adenylyl-removing domain of glutamate-ammonia-ligase adenylyltransferase (ΔglnE-AR_KO1). | ΔnifL::Prm5 ΔglnE-AR_KO1 | SEQ ID NO: 54 | SEQ ID NO: 61 |
| CM014 | Mutant of CI006 | Disruption of nifL gene with a fragment of the region upstream of the 1pp gene and the first 29bp of the 1pp gene inserted (Prm1). | ΔnifL::Prm1 | SEQ ID NO: 55 | |
| CM011 | Mutant of CI019 | Disruption of nifL gene with a spectinomycin resistance expression cassette (SpecR) encoding the streptomycin 3″-O-adenylyltransferase gene aadA inserted. | ΔnifL::SpecR | SEQ ID NO: 56 | |
| CM011 | Mutant of CI019 | Disruption of nifL gene with a spectinomycin resistance expression cassette (SpecR) encoding the streptomycin 3″-O-adenylyltransferase gene aadA inserted. | ΔnifL::SpecR | SEQ ID NO: 57 | |
| CM013 | Mutant of CI006 | Disruption of nifL gene with a kanamycin resistance expression cassette (KanR) encoding the aminoglycoside O-phosphotransferase gene aph1 inserted. | ΔnifL::KanR | SEQ ID NO: 58 | |
| CM011 | Mutant of CI019 | Disruption of nifL gene with a spectinomycin resistance expression cassette (SpecR) encoding the streptomycin 3″-O-adenylyltransferase gene aadA inserted. | ΔnifL::SpecR | SEQ ID NO: 59 | |
| CM011 | Mutant of CI019 | Disruption of nifL gene with a spectinomycin resistance expression cassette (SpecR) encoding the streptomycin 3″-O-adenylyltransferase gene aadA inserted. | ΔnifL::SpecR | SEQ ID NO: 60 | |

### Commodity and Insurance Transactions

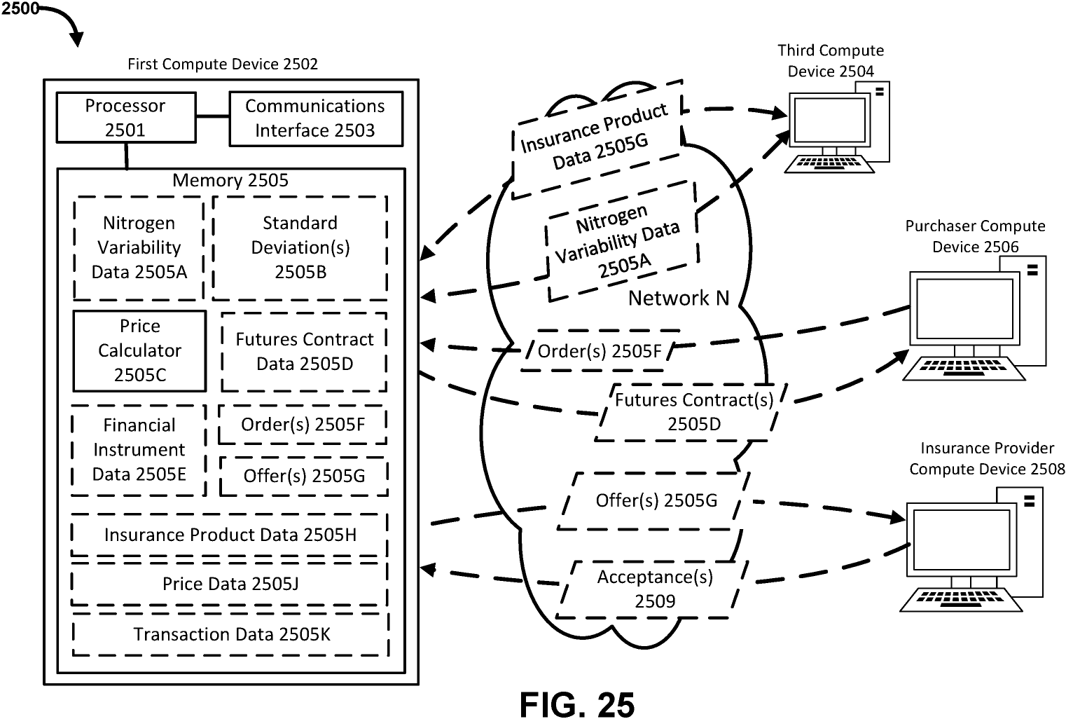

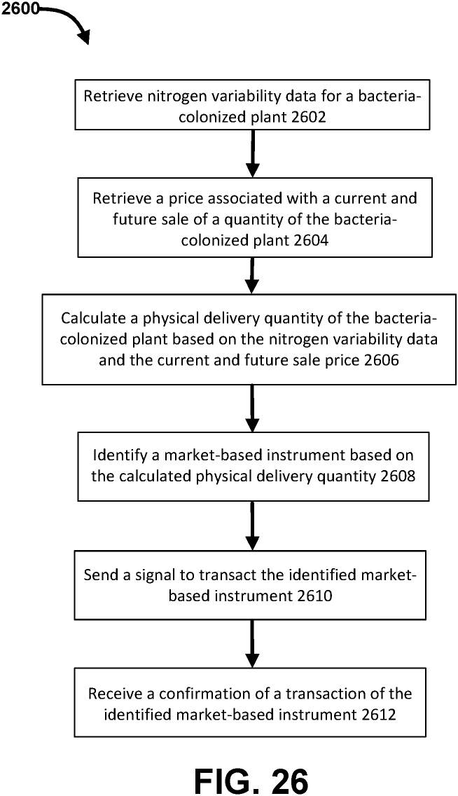

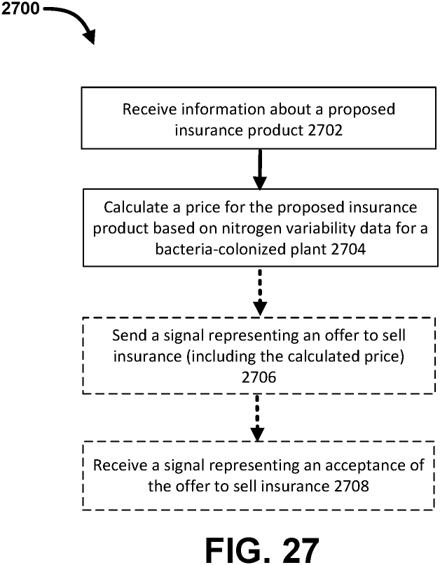

FIG. 25 is a system diagram for the transacting of financial and insurance instruments, according to some embodiments. As shown in FIG. 25, the system 2500 includes a first compute device 2502, a third (e.g., remote) compute device 2504, optionally a purchaser compute device 2506, and optionally an insurance provider compute device 2508. Each of the first compute device 2502, third compute device 2504, purchaser compute device 2506, and insurance provider compute device 2508 can be in wired or wireless communication with another, for example via a telecommunications network (shown as “Network N” in FIG. 25). The first compute device 2502 includes a processor 2501 that is operably coupled to a memory 2505 and to a communications interface (e.g., a wireless antenna) 2503. The memory 2505 can store data and/or processor-executable code containing instructions to perform actions, for example as shown and described herein and with reference to FIGS. 26-27. As shown in FIG. 25, the memory 2505 of the first compute device 2502 can include one or more of: nitrogen variability data 2505A (e.g., whole plant nitrogen variability data), standard deviation(s) 2505B, a price calculator 2505C, futures contract data 2505D, financial instrument data 2505E, order(s) 2505F, offer(s) 2505G, insurance product data 2505H, price data 2505J, and transaction data 2505K. Any of the data stored in the memory 2505 (e.g., the nitrogen variability data 2505A, standard deviation(s) 2505B, futures contract data 2505D, financial instrument data 2505E, order(s) 2505F, offer(s) 2505G, insurance product data 2505H, price data 2505J, and transaction data 2505K) can be generated locally (i.e., at the first compute device 2502 and/or using the processor 2501) and/or can be received at the first compute device 2502 (and using the communications interface 2503 thereof) from one or more remote compute devices. For example, as shown in FIG. 25, the insurance product data 2505G and/or the nitrogen variability data 2505A can be received, via the network N, from the third comptue device 2504. Such data is optionally received at the first compute device 2502 in response to a request to retrieve such data, or a query for such data, sent from the first comptue device 2502 to the remote (e.g., third) compute device. In some implementations, order(s) 2505F can be received at the first compute device 2502 (via the network N and using the communications interface 2503) the purchaser compute device 2506, and in response to receiving and approving the order(s) 2505F, one or more futures contract(s) 2505D can be sent from the first compute device 2502, via the network N, to the purchaser compute device 2506. Alternatively, or in addition, in some implementations, offer(s) 2505G can be sent from the first compute device 2502 (via the network N and using the communications interface 2503) to the insurance provider compute device 2506, and in response to receiving and approving the offer(s) 2505G, a signal representing an acceptance of the offer(s), 509, can be sent from the insurance provider compute device 2506 back to the first comptue device 2502, via the network N.

FIG. 26 is a flow diagram illustrating a method for determining a quantity of a crop plant to sell based on a whole plant nitrogen variability of a bacteria-colonized plant (e.g., a corn plant), according to some embodiments. The method of FIG. 26 can be implemented, for example, using the system 2500 of FIG. 25. As shown in FIG. 26, the method 2600 includes retrieving, at 2602, via a processor and from a database (e.g., including corn nitrogen variability data) operably coupled to the processor, nitrogen variability data for a bacteria-colonized plant. The nitrogen variability data includes a whole plant nitrogen variability that is lower than a whole plant nitrogen variability of a plant that has not been bacterially colonized. The whole plant nitrogen variability of the bacteria-colonized plant can be at least about 15% lower than (e.g., about 17% lower than, or between about 17% and about 20% lower than) a whole plant nitrogen variability of a corresponding or similar plant that has not been bacterially colonized. The processor retrieves, at 2604, from a database operably coupled to the processor, a price associated with a current and future sale of a quantity of the bacteria-colonized plant. At 2606, the processor calculates a physical delivery quantity of the bacteria-colonized plant based on the nitrogen variability data for the bacteria-colonized plant and the current sale price and future sale price. The physical delivery quantity of the bacteria-colonized plant can be or include a predicted physical delivery quantity of the bacteria-colonized plant (e.g., a predicted quantity of bacteria-colonized plants grown on land that has historically exhibited higher whole plant nitrogen variability in plants that were not bacterially colonized).

The method 2600 also includes, at 2608, identifying a market-based instrument based on the calculated physical delivery quantity of the bacteria-colonized plant, and at 2610, sending, via the processor, a signal representing an instruction to transact the identified market-based instrument (e.g., within a secondary market). The market-based instrument can be or include, for example, a forward contract, a futures contract, an options contract, and/or a commodity swap contract. The instruction to transact the identified market-based instrument can include a trading symbol. At 2612, the processor receives, in response to sending the instruction to transact the identified market-based instrument, a signal representing a confirmation of a transaction of the identified market-based instrument. The signal representing the confirmation of the transaction of the identified market-based instrument can be received at the processor via an application programming interface (API).

In some implementations of the method 2600, the calculating the physical delivery quantity is performed prior to a growing season associated with the bacteria-colonized plant. Alternatively or in addition, the transaction of the identified market-based instrument is performed prior to a growing season associated with the bacteria-colonized plant. The method 2600 optionally also includes producing the physical delivery quantity of the bacteria-colonized plant.

In some embodiments, producing the bacteria-colonized plant of method 2600 includes (1) providing to a locus a plurality of non-intergeneric remodeled bacteria that each produce fixed N of at least about 5.49 × 10⁻¹³ mmol of N per CFU per hour, and (2) providing to the locus the pre-colonization plant.

In some embodiments, the bacteria-colonized plant is produced using biological nitrogen fixation and/or an engineered N fixing microbe.

In some embodiments, the bacteria-colonized plant is produced using a microorganism capable of fixing atmospheric nitrogen for associated crops.

FIG. 27 is a flow diagram illustrating a method for pricing and transacting an insurance product, based on nitrogen variability data for a bacteria-colonized plant (e.g., a corn plant), according to some embodiments. The method of FIG. 26 can be implemented, for example, using the system 2500 of FIG. 25. As shown in FIG. 27, the method 2700 includes receiving, at 2702, via a processor, information about a proposed insurance product. At 2704, the processor calculates a price for the proposed insurance product based on a whole plant nitrogen variability for a bacteria-colonized plant. The nitrogen variability data for the bacteria-colonized plant includes a whole plant nitrogen variability that is lower than a whole plant nitrogen variability of a plant that has not been bacterially colonized. Optionally, the method 2700 also includes, at 2706, the processor sending, from a compute device of a seller, a signal representing an offer to sell insurance. The offer to sell insurance includes the calculated price for the proposed insurance product. The signal representing the offer to sell insurance can be sent via an application programming interface (API). The signal representing the offer to sell insurance optionally also includes the whole plant nitrogen variability for the bacteria-colonized plant. The method 2700 can also optionally include receiving, at the processor (at 2708), in response to sending the calculated price for the proposed insurance product, a signal representing an acceptance of the offer to sell insurance. The signal representing acceptance of the offer to sell insurance can be received via an API.

In some embodiments, the calculating the price for the proposed insurance product is performed prior to a growing season associated with the bacteria-colonized plant. Alternatively or in addition, the sending the signal representing the offer to sell insurance is performed prior to a growing season associated with the bacteria-colonized plant.

In some embodiments, the nitrogen variability data is based on a production of the bacteria-colonized plant by a process comprising (1) providing to a locus a plurality of non-intergeneric remodeled bacteria that each produce fixed N of at least about 5.49 × 10⁻¹³ mmol of N per CFU per hour, and (2) providing to the locus a pre-colonization plant.

In some embodiments, the method 2700 also includes producing the bacteria-colonized plant, using a pre-colonization plant, by: (1) providing to a locus a plurality of non-intergeneric remodeled bacteria that each produce fixed N of at least about 5.49 × 10⁻¹³ mmol of N per CFU per hour, and (2) providing to the locus the pre-colonization plant.

In some embodiments, the nitrogen variability data is based on one or more of: producing the bacteria-colonized plant by a process comprising using an engineered N fixing microbe, producing the bacteria-colonized plant by a process comprising using biological nitrogen fixation, or producing the bacteria-colonized plant by a process comprising using a microorganism capable of fixing atmospheric nitrogen for associated crops.

In some embodiments, a method of increasing the value of a commodity (e.g., a crop plant, such as corn) includes decreasing variability in whole plant nitrogen of the commodity by growing the commodity in the presence of a nutrient-providing microorganism. The method optionally also includes determining a plurality of different prices for sale of the commodity, for each of multiple markets in which the commodity can be sold. The variability in whole plant nitrogen of the commodity can comprise, for example, variability in nitrogen variability of the commodity across a farmer’s field. Alternatively or in addition, the variability in whole plant nitrogen of the commodity can be substantially due to variability in response to weather conditions. Decreasing the variability in whole plant nitrogen of the commodity can allow a seller of the commodity to increase sales of the commodity into markets with higher pricing for the commodity, or allow the seller of the commodity to decrease sales of the commodity into markets with lower pricing for the commodity.

In some embodiments, the markets with higher pricing for the commodity comprise markets that occur prior to a production season for the commodity.

In some embodiments, the markets with lower pricing for the commodity comprise markets that occur after a production season for the commodity.

In some embodiments, growing the crop plant in the presence of the nutrient-providing microorganism improves the availability of the provided one or more nutrients to the crop plant. The one or more nutrients can include, for example, nitrogen, and the microorganism can be a nitrogen-fixing bacterium.

In some embodiments, a method of decreasing insurance costs for a commodity (e.g., a crop plant, such as corn) includes decreasing variability in whole plant nitrogen of the commodity by growing the commodity in the presence of a nutrient-providing microorganism. The variability in whole plant nitrogen of the commodity can include, for example, variability in whole plant nitrogen of the commodity across a farmer’s field. Alternatively or in addition, the variability in whole plant nitrogen of the commodity can be substantially due to variability in response to weather conditions. Growing the crop plant in the presence of the nutrient-providing microorganism can improve the availability of the provided one or more nutrients to the crop plant. The one or more nutrients can include, for example, nitrogen, and the microorganism can be a nitrogen-fixing bacterium.

The term “automatically” is used herein to modify actions that occur without direct input or prompting by an external source such as a user. Automatically occurring actions can occur periodically, sporadically, in response to a detected event (e.g., a user logging in), or according to a predetermined schedule.

The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

The term “processor” should be interpreted broadly to encompass a general purpose processor, a central processing unit (CPU), a microprocessor, a digital signal processor (DSP), a controller, a microcontroller, a state machine and so forth. Under some circumstances, a “processor” may refer to an application specific integrated circuit (ASIC), a programmable logic device (PLD), a field programmable gate array (FPGA), etc. The term “processor” may refer to a combination of processing devices, e.g., a combination of a DSP and a microprocessor, a plurality of microprocessors, one or more microprocessors in conjunction with a DSP core or any other such configuration.

The term “memory” should be interpreted broadly to encompass any electronic component capable of storing electronic information. The term memory may refer to various types of processor-readable media such as random access memory (RAM), read-only memory (ROM), non-volatile random access memory (NVRAM), programmable read-only memory (PROM), erasable programmable read only memory (EPROM), electrically erasable PROM (EEPROM), flash memory, magnetic or optical data storage, registers, etc. Memory is said to be in electronic communication with a processor if the processor can read information from and/or write information to the memory. Memory that is integral to a processor is in electronic communication with the processor.

The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may comprise a single computer-readable statement or many computer-readable statements.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

Some embodiments and/or methods described herein can be performed by software (executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including C, C++, Java™, Ruby, Visual Basic™, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

Various concepts may be embodied as one or more methods, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Put differently, it is to be understood that such features may not necessarily be limited to a particular order of execution, but rather, any number of threads, processes, services, servers, and/or the like that may execute serially, asynchronously, concurrently, in parallel, simultaneously, synchronously, and/or the like in a manner consistent with the disclosure. As such, some of these features may be mutually contradictory, in that they cannot be simultaneously present in a single embodiment. Similarly, some features are applicable to one aspect of the innovations, and inapplicable to others.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The indefinite articles “a” and “an,” as used herein in the specification and in the embodiments, unless clearly indicated to the contrary, should be understood to mean “at least one.”

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the embodiments, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the U.S. Pat. Office Manual of Patent Examining Procedures, Section 2111.03.

### Commodity and Insurance Pricing

Applicants have discovered that provision of nutrients to crop plants using associative microbes can unexpectedly decrease whole plant nitrogen variability that can result from heterogeneity in field conditions (e.g., differences in soil types or differences resuting from weather conditions). For example, provision of nitrogen through nitrogen fixing microbes, including remodeled microbes of the disclosure allow a farmer to more accurately predict the whole plant nitrogen variability and yield that will result from a given set of acreage. Beacause the remodeled microbes lead to a significantly lower distribution around whole plant nitrogen variability, i.e. a lower standard deviation, the farmer can more accurately predict what his expected yield results shall be across his land. This is true irrespective of the soil types or weather conditions the farmer may face in a given growing season, as the remodeled microbial product stays with the plant and provides the plant with N throughout the season, even in bad weather or problematic soils. The reduced whole plant nitrogen variability, and the resulting increased predicatability/reliability around yield results, across all of a farmer’s acreage, allows the farmer to be more aggressive in taking out purchase options at the beginning of the season, as the farmer will have confidence that he can meet demand on a given contract date. This results in the farmer not having to go to the “cash market” during season with his product, because they were able to confidently market their crop pre-season, based on the knowledge/data that, by providing nitrogen to the crops through the use of the taught microbial product, they could obtain a tight distribution around whole plant nitrogen variability, i.e. increased predictability of nitrogen levels and less whole plant nitrogen variability across a field. In turn, by using associative microbes for the provision of crop nutrients, such as the use of the remodeled nitrogen fixing microbes disclosed herein, a farmer is expected to obtain greater value from their harvested crop than crop produced using traditionally applied nutrients, e.g., application of traditional nitrogen fertilizer alone, for which whole plant nitrogen variability can be greater.

Another result of the reduced whole plant nitrogen variability seen with utilization of associative microbes, such as the taught microbes, is a decrease in the price of Actual Production History (APH) insurance contracts. These insurance policies insure producers against yield losses due to natural causes (e.g. Acts of God, drought, excessive moisture, hail, wind, frost, insects, disease, etc.). The producer selects the amount of average yield to insure (e.g. 50-75% or perhaps up to 85% percent). The producer also selects the percent of the predicted price to insure, between 55 and 100 percent of the crop price established annually by RMA. If the harvested plus any appraised production is less than the yield insured, the producer is paid an indemnity based on the difference. Indemnities are calculated by multiplying this difference by the insured percentage of the price selected when crop insurance was purchased and by the insured share. Because the remodeled microbes of the disclosure reduce whole plant nitrogen variability, an insurer is able to more accurately and confidently predict that there will not be any delta between the harvested yield and the yield insured at the start of the growing season.

Example microbes of the present disclosure (e.g. WT and remodeled non-intergeneric) are listed in Table 4. If a microbe is engineered, then the corresponding genetic change will be noted in the description of the below table. Further, engineered strains are named with the WT strain ID number (e.g. 137) followed by the engineered variant number with a dash (e.g. 137-1036).

TABLE 4

WT and Remodeled Non-intergeneric Microbes of the Disclosure

| Strain | Strain ID | SEQ ID NO | Genotype | Description |
|---|---|---|---|---|
| CI63; CI063 | 63 | SEQ ID NO 85 | 16S | N/A |
| CI63; CI063 | 63 | SEQ ID NO 86 | nifH | N/A |
| CI63; CI063 | 63 | SEQ ID NO 87 | nifD1 | 1 of 2 unique genes annotated as nifD in 63 genome |
| CI63; CI063 | 63 | SEQ ID NO 88 | nifD2 | 2 of 2 unique genes annotated as nifD in 63 genome |
| CI63; CI063 | 63 | SEQ ID NO 89 | nifK1 | 1 of 2 unique genes annotated as nifK in 63 genome |
| CI63; CI063 | 63 | SEQ ID NO 90 | nifK2 | 2 of 2 unique genes annotated as nifK in 63 genome |
| CI63; CI063 | 63 | SEQ ID NO 91 | nifL | N/A |
| CI63; CI063 | 63 | SEQ ID NO 92 | nifA | N/A |
| CI63; CI063 | 63 | SEQ ID NO 93 | glnE | N/A |
| CI63; CI063 | 63 | SEQ ID NO 94 | amtB | N/A |
| CI63; CI063 | 63 | SEQ ID NO 95 | PinfC | 500bp immediately upstream of the ATG start codon of the infC gene |
| CI137 | 137 | SEQ ID NO 96 | 16S | N/A |
| CI137 | 137 | SEQ ID NO 97 | nifH1 | 1 of 2 unique genes annotated as nifH in 137 genome |
| CI137 | 137 | SEQ ID NO 98 | nifH2 | 2 of 2 unique genes annotated as nifH in 137 genome |
| CI137 | 137 | SEQ ID NO 99 | nifD1 | 1 of 2 unique genes annotated as nifD in 137 genome |
| CI137 | 137 | SEQ ID NO 100 | nifD2 | 2 of 2 unique genes annotated as nifD in 137 genome |
| CI137 | 137 | SEQ ID NO 101 | nifK1 | 1 of 2 unique genes annotated as nifK in 137 genome |
| CI137 | 137 | SEQ ID NO 102 | nifK2 | 2 of 2 unique genes annotated as nifK in 137 genome |
| CI137 | 137 | SEQ ID NO 103 | nifL | N/A |
| CI137 | 137 | SEQ ID NO 104 | nifA | N/A |
| CI137 | 137 | SEQ ID NO 105 | glnE | N/A |
| CI137 | 137 | SEQ ID NO 106 | PinfC | 500bp immediately upstream of the TTG start codon of infC |
| CI137 | 137 | SEQ ID NO 107 | amtB | N/A |
| CI137 | 137 | SEQ ID NO 108 | Prm8.2 | internal promoter located in nlpI gene; 299bp starting at 81bp after the A of the ATG of the nlpI gene |
| CI137 | 137 | SEQ ID NO 109 | Prm6.2 | 300bp upstream of the secE gene starting at 57bp upstream of the A of the ATG of secE |
| CI137 | 137 | SEQ ID NO 110 | Prm1.2 | 400bp immediately upstream of the ATG of cspE gene |
| none | 728 | SEQ ID NO 111 | 16S | N/A |
| none | 728 | SEQ ID NO 112 | nifH | N/A |
| none | 728 | SEQ ID NO 113 | nifD1 | 1 of 2 unique genes annotated as nifD in 728 genome |
| none | 728 | SEQ ID NO 114 | nifD2 | 2 of 2 unique genes annotated as nifD in 728 genome |
| none | 728 | SEQ ID NO 115 | nifK1 | 1 of 2 unique genes annotated as nifK in 728 genome |
| none | 728 | SEQ ID NO 116 | nifK2 | 2 of 2 unique genes annotated as nifK in 728 genome |
| none | 728 | SEQ ID NO 117 | nifL | N/A |
| none | 728 | SEQ ID NO 118 | nifA | N/A |
| none | 728 | SEQ ID NO 119 | glnE | N/A |
| none | 728 | SEQ ID NO 120 | amtB | N/A |
| none | 850 | SEQ ID NO 121 | 16S | N/A |
| none | 852 | SEQ ID NO 122 | 16S | N/A |
| none | 853 | SEQ ID NO 123 | 16S | N/A |
| none | 910 | SEQ ID NO 124 | 16S | N/A |
| none | 910 | SEQ ID NO 125 | nifH | N/A |
| none | 910 | SEQ ID NO 126 | Dinitrogenase iron-molybdentun cofactor CDS | N/A |
| none | 910 | SEQ ID NO 127 | nifD1 | N/A |
| none | 910 | SEQ ID NO 128 | nifD2 | N/A |
| none | 910 | SEQ ID NO 129 | nifK1 | N/A |
| none | 910 | SEQ ID NO 130 | nifK2 | N/A |
| none | 910 | SEQ ID NO 131 | nifL | N/A |
| none | 910 | SEQ ID NO 132 | nifA | N/A |
| none | 910 | SEQ ID NO 133 | glnE | N/A |
| none | 910 | SEQ ID NO 134 | amtB | N/A |
| none | 910 | SEQ ID NO 135 | PinfC | 498bp immediately upstream of the ATG of the infC gene |
| none | 1021 | SEQ ID NO 136 | 16S | N/A |
| none | 1021 | SEQ ID NO 137 | nifH | N/A |
| none | 1021 | SEQ ID NO 138 | nifD1 | 1 of 2 unique genes annotated as nifD in 910 genome |
| none | 1021 | SEQ ID NO 139 | nifD2 | 2 of 2 unique genes annotated as nifD in 910 genome |
| none | 1021 | SEQ ID NO 140 | nifK1 | 1 of 2 unique genes annotated as nifK in 910 genome |
| none | 1021 | SEQ ID NO 141 | nifK2 | 2 of 2 unique genes annotated as nifK in 910 genome |
| none | 1021 | SEQ ID NO 142 | nifL | N/A |
| none | 1021 | SEQ ID NO 143 | nifA | N/A |
| none | 1021 | SEQ ID NO 144 | glnE | N/A |
| none | 1021 | SEQ ID NO 145 | amtB | N/A |
| none | 1021 | SEQ ID NO 146 | PinfC | 500bp immediately upstream of the ATG start codon of the infC gene |
| none | 1021 | SEQ ID NO 147 | Prm1 | 348bp includes the 319bp immediately upstream of the ATG start codon of the Ipp gene and the first 29bp of the Ipp gene |
| none | 1021 | SEQ ID NO 148 | Prm7 | 339bp upstream of the sspA gene, ending at 46bp upstream of the ATG of the sspA gene |
| none | 1113 | SEQ ID NO 149 | 16S | N/A |
| none | 1113 | SEQ ID NO 150 | nifH | N/A |
| none | 1113 | SEQ ID NO 151 | nifD1 | 1 of 2 unique genes annotated as nifD in 1113 genome |
| none | 1113 | SEQ ID NO 152 | nifD2 | 2 of 2 unique genes annotated as nifD in 1113 genome |
| none | 1113 | SEQ ID NO 153 | nifK | N/A |
| none | 1113 | SEQ ID NO 154 | nifL | N/A |
| none | 1113 | SEQ ID NO 155 | nifA partial gene | due to a gap in the sequence assembly, we can only identify a partial gene from the 1113 genome |
| none | 1113 | SEQ ID NO 156 | glnE | N/A |
| none | 1116 | SEQ ID NO 157 | 16S | |
| none | 1116 | SEQ ID NO 158 | nifH | |
| none | 1116 | SEQ ID NO 159 | nifD1 | 1 of 2 unique genes annotated as nifD in 1116 genome |
| none | 1116 | SEQ ID NO 160 | nifD2 | 2 of 2 unique genes annotated as nifD in 1116 genome |
| none | 1116 | SEQ ID NO 161 | nifK1 | 1 of 2 unique genes annotated as nifK in 1116 genome |
| none | 1116 | SEQ ID NO 162 | nifK2 | 2 of 2 unique genes annotated as nifK in 1116 genome |
| none | 1116 | SEQ ID NO 163 | nifL | N/A |
| none | 1116 | SEQ ID NO 164 | nifA | N/A |
| none | 1116 | SEQ ID NO 165 | glnE | N/A |
| none | 1116 | SEQ ID NO 166 | amtB | N/A |
| none | 1293 | SEQ ID NO 167 | 16S | N/A |
| none | 1293 | SEQ ID NO 168 | nifH | N/A |
| none | 1293 | SEQ ID NO 169 | nifD1 | 1 of 2 unique genes annotated as nifD in 1293 genome |
| none | 1293 | SEQ ID NO 170 | nifD2 | 2 of 2 unique genes annotated as nifD in 1293 genome |
| none | 1293 | SEQ ID NO 171 | nifK | 1 of 2 unique genes annotated as nifK in 1293 genome |
| none | 1293 | SEQ ID NO 172 | nifK1 | 2 of 2 unique genes annotated as nifK in 1293 genome |
| none | 1293 | SEQ ID NO 173 | nifA | N/A |
| none | 1293 | SEQ ID NO 174 | glnE | N/A |
| none | 1293 | SEQ ID NO 175 | amtB1 | 1 of 2 unique genes annotated as amtB in 1293 genome |
| none | 1293 | SEQ ID NO 176 | amtB2 | 2 of 2 unique genes annotated as amtB in 1293 genome |
| none | 1021-1612 | SEQ ID NO 177 | ΔnifL::PinfC | starting at 24bp after the A of the ATG start codon, 1375bp of nifL have been deleted and replaced with the 1021 PinfC promoter sequence |
| none | 1021-1612 | SEQ ID NO 178 | ΔnifL::PinfC with 500bp flank | starting at 24bp after the A of the ATG start codon, 1375bp of nifL have been deleted and replaced with the 1021 PinfC promoter sequence; 500bp flanking the nifL gene upstream and downstream are included |
| none | 1021-1612 | SEQ ID NO 179 | glnEΔAR-2 | glnE gene with 1673bp immediately downstream of the ATG start codon deleted, resulting in a truncated glnE protein lacking the adenylyl-removing (AR) domain |
| none | 1021-1612 | SEQ ID NO 180 | glnEΔAR-2 with 500bp flank | glnE gene with 1673bp immediately downstream of the ATG start codon deleted, resulting in a truncated glnE protein lacking the adenylyl-removing (AR) domain; 500bp flanking the glnE gene upstream and downstream are included |
| none | 1021-1615 | SEQ ID NO 181 | ΔnifL::Prm1 | starting at 24bp after the A of the ATG start codon, 1375bp of nifL have been deleted and replaced with the 1021 Prm1 promoter sequence |
| none | 1021-1615 | SEQ ID NO 182 | ΔnifL::Prm1 with 500bp flank | starting at 24bp after the A of the ATG start codon, 1375bp of nifL have been deleted and replaced with the 1021 rm1 promoter sequence; 500bp flanking the nifL gene upstream and downstream are included |
| none | 1021-1615 | SEQ ID NO 183 | glnEΔAR-2 | glnE gene with 1673bp immediately downstream of the ATG start codon deleted, resulting in a truncated glnE protein lacking the adenylyl-removing (AR) domain |
| none | 1021-1615 | SEQ ID NO 184 | glnEΔAR-2 with 500bp flank | glnE gene with 1673bp immediately downstream of the ATG start codon deleted, resulting in a truncated glnE protein lacking the adenylyl-removing (AR) domain; 500bp flanking the glnE gene upstream and downstream are included |
| none | 1021-1619 | SEQ ID NO 185 | ΔnifL::Prm1 | starting at 24bp after the A of the ATG start codon, 1375bp of nifL have been deleted and replaced with the 1021 Prm1 promoter sequence |
| none | 1021-1619 | SEQ ID NO 186 | ΔnifL::Prm1 with 500bp flank | starting at 24bp after the A of the ATG start codon, 1375bp of nifL have been deleted and replaced with the 1021 rm1 promoter sequence; 500bp flanking the nifL gene upstream and downstream are included |
| none | 1021-1623 | SEQ ID NO 187 | glnEΔAR-2 | glnE gene with 1673bp immediately downstream of the ATG start codon deleted, resulting in a truncated glnE protein lacking the adenylyl-removing (AR) domain |
| none | 1021-1623 | SEQ ID NO 188 | glnEAAR-2 with 500bp flank | glnE gene with 1673bp immediately downstream of the ATG start codon deleted, resulting in a truncated glnE protein lacking the adenylyl-removing (AR) domain; 500bp flanking the glnE gene upstream and downstream are included |
| none | 1021-1623 | SEQ ID NO 189 | ΔnifL::Prm7 | starting at 24bp after the A of the ATG start codon, 1375bp of nifL have been deleted and replaced with the 1021 Prm7 promoter sequence |
| none | 1021-1623 | SEQ ID NO 190 | ΔnifL::Prm7 with 500bp flank | starting at 24bp after the A of the ATG start codon, 1375bp of nifL. have been deleted and replaced with the 1021 rm7 promoter sequence; 500bp flanking the nifL gene upstream and downstream are included |
| none | 137-1034 | SEQ ID NO 191 | glnEΔAR-2 | glnE gene with 1290bp immediately downstream of the ATG start codon deleted, resulting in a truncated glnE protein lacking the adenylyl-removing (AR) domain |
| none | 137-1034 | SEQ ID NO 192 | glnEAAR-2 with 500bp flank | glnE gene with 1290bp immediately downstream of the ATG start codon deleted, resulting in a truncated glnE protein lacking the adenylyl-removing (AR) domain; 500bp flanking the glnE gene upstream and downstream are included |
| none | 137-1036 | SEQ ID NO 193 | ΔnifL::PinfC | starting at 24bp after the A of the ATG start codon, 1372bp of nifL have been deleted and replaced with the 137 PinfC promoter sequence |
| none | 137-1036 | SEQ ID NO 194 | ΔnifL::PinfC with 500bp flank | starting at 24bp after the A of the ATG start codon, 1372bp of nifL have been deleted and replaced with the 137 PinfC promoter sequence; 500bp flanking the nifL gene upstream and downstream are included |
| none | 137-1314 | SEQ ID NO 195 | glnEΔAR-2 36bp deletion | glnE gene with 1290bp immediately downstream of the ATG start codon deleted AND 36bp deleted beginning at 1472bp downstream of the start codon, resulting in a truncated glnE protein lacking the adenylyl-removing (AR) domain |
| none | 137-1314 | SEQ ID NO 196 | glnEΔAR-2 36bp deletion | glnE gene with 1290bp immediately downstream of the ATG start codon deleted AND 36bp deleted beginning at 1472bp downstream of the start codon, resulting in a truncated glnE protein lacking the adenylyl-removing (AR) domain; 500bp flanking the nifL gene upstream and downstream are included |
| none | 137-1314 | SEQ ID NO 197 | ΔnifL::Prm8.2 | starting at 24bp after the A of the ATG start codon, 1372bp of nifL have been deleted and replaced with the 137 Prm8.2 promoter sequence |
| none | 137-1314 | SEQ ID NO 198 | ΔnifL::Prm8.2 with 500bp flank | starting at 24bp after the A of the ATG start codon, 1372bp of nifL have been deleted and replaced with the 137 Prm8.2 promoter sequence; 500bp flanking the nifL gene upstream and downstream are included |
| none | 137-1329 | SEQ ID NO 199 | glnEΔAR-2 36bp deletion | glnE gene with 1290bp immediately downstream of the ATG start codon deleted AND 36bp deleted beginning at 1472bp downstream of the start codon, resulting in a truncated glnE protein lacking the adenylyl-removing (AR) domain |
| none | 137-1329 | SEQ ID NO 200 | glnEΔAR-2 36bp deletion | glnE gene with 1290bp immediately downstream of the ATG start codon deleted AND 36bp deleted beginning at 1472bp downstream of the start codon, resulting in a truncated glnE protein lacking the adenylyl-removing (AR) domain; 500bp flanking the nifL gene upstream and downstream are included |
| none | 137-1329 | SEQ ID NO 201 | ΔnifL::Prm6.2 | starting at 24bp after the A of the ATG start codon, 1372bp of nifL have been deleted and replaced with the 137 Prm6.2 promoter sequence |
| none | 137-1329 | SEQ ID NO 202 | ΔnifL::Prm6.2 with 500bp flank | starting at 24bp after the A of the ATG start codon, 1372bp of nifL have been deleted and replaced with the 137 Prm6.2 promoter sequence; 500bp flanking the nifL gene upstream and downstream are included |
| none | 137-1382 | SEQ ID NO 203 | ΔnifL::Prm1.2 | starting at 24bp after the A of the ATG start codon, 1372bp of nifL have been deleted and replaced with the 137 Prm1.2 promoter sequence |
| none | 137-1382 | SEQ ID NO 204 | ΔnifL::Prm1.2 with 500bp flank | starting at 24bp after the A of the ATG start codon, 1372bp of nifL have been deleted and replaced with the 137 Prm1.2 promoter sequence; 500bp flanking the nifL gene upstream and downstream are included |
| none | 137-1382 | SEQ ID NO 205 | glnEΔAR-2 36bp deletion | glnE gene with 1290bp immediately downstream of the ATG start codon deleted AND 36bp deleted beginning at 1472bp downstream of the start codon, resulting in a truncated glnE protein lacking the adenylyl-removing (AR) domain |
| none | 137-1382 | SEQ ID NO 206 | glnEAAR-2 36bp deletion | glnE gene with 1290bp immediately downstream of the ATG start codon deleted AND 36bp deleted beginning at 1472bp downstream of the start codon, resulting in a truncated glnE protein lacking the adenylyl-removing (AR) domain; 500bp flanking the nifL gene upstream and downstream are included |
| none | 137-1586 | SEQ ID NO 207 | ΔnifL::PinfC | starting at 24bp after the A of the ATG start codon, 1372bp of nifL have been deleted and replaced with the 137 PinfC promoter sequence |
| none | 137-1586 | SEQ ID NO 208 | AnifL::PinfC with 500bp flank | starting at 24bp after the A of the ATG start codon, 1372bp of nifL have been deleted and replaced with the 137 PinfC promoter sequence; 500bp flanking the nifL gene upstream and downstream are included |
| none | 137-1586 | SEQ ID NO 209 | glnEΔAR-2 | glnE gene with 1290bp immediately downstream of the ATG start codon deleted, resulting in a truncated glnE protein lacking the adenylyl-removing (AR) domain |
| none | 137-1586 | SEQ ID NO 210 | glnEAAR-2 with 500bp flank | glnE gene with 1290bp immediately downstream of the ATG start codon deleted, resulting in a truncated glnE protein lacking the adenylyl-removing (AR) domain; 500bp flanking the glnE gene upstream and downstream are included |
| none | 19-594 | SEQ ID NO 211 | glnEΔAR-2 | glnE gene with 1650bp immediately downstream of the ATG start codon deleted, resulting in a truncated glnE protein lacking the adenylyl-removing (AR) domain |
| none | 19-594 | SEQ ID NO 212 | glnEΔAR-2 with 500bp flank | glnE gene with 1650bp immediately downstream of the ATG start codon deleted, resulting in a truncated glnE protein lacking the adenylyl-removing (AR) domain; 500bp flanking the glnE gene upstream and downstream are included |
| none | 19-594 | SEQ ID NO 213 | ΔnifL::Prm6.1 | starting at 221bp after the A of the ATG start codon, 845bp of nifL have been deleted and replaced with the CI019 Prm6.1 promoter sequence |
| none | 19-594 | SEQ ID NO 214 | ΔnifL::Prm6.1 with 500bp flank | starting at 221bp after the A of the ATG start codon, 845bp of nifL have been deleted and replaced with the CI019 Prm6.1promoter sequence; 500bp flanking the nifL gene upstream and downstream are included |
| none | 19-714 | SEQ ID NO 215 | ΔnifL::Prm6.1 | starting at 221bp after the A of the ATG start codon, 845bp of nifL have been deleted and replaced with the CI019 Prm6.1 promoter sequence |
| none | 19-714 | SEQ ID NO 216 | ΔnifL::Prm6.1 with 500bp flank | starting at 221bp after the A of the ATG start codon, 845bp of nifL have been deleted and replaced with the CI019 Prm6.1promoter sequence: 500bp flanking the nifL gene upstream and downstream are included |
| none | 19-715 | SEQ ID NO 217 | ΔnifL::Prm7.1 | starting at 221bp after the A of the ATG start codon, 845bp of nifL have been deleted and replaced with the CI019 Prm7.1 promoter sequence |
| none | 19-715 | SEQ ID NO 218 | ΔnifL::Prm7.1 with 500bp flank | starting at 221bp after the A of the ATG start codon, 845bp of nifL have been deleted and replaced with the CI019 Prm76.1promoter sequence; 500bp flanking the nifL gene upstream and downstream are included |
| 19-713 | 19-750 | SEQ ID NO 219 | ΔnifL::Prm1.2 | starting at 221bp after the A of the ATG start codon, 845bp of nifL have been deleted and replaced with the CI019 Prm1.2 promoter sequence |
| 19-713 | 19-750 | SEQ ID NO 220 | ΔnifL::Prm1.2 with 500bp flank | starting at 221bp after the A of the ATG start codon, 845bp of nifL have been deleted and replaced with the CI019 Prm1.2 promoter sequence; 500bp flanking the nifL gene upstream and downstream are included |
| 17-724 | 19-804 | SEQ ID NO 221 | ΔnifL::Prm1.2 | starting at 221bp after the A of the ATG start codon, 845bp of nifL have been deleted and replaced with the CI019 Prm1.2 promoter sequence |
| 17-724 | 19-804 | SEQ ID NO 222 | ΔnifL::Prm1.2 with 500bp flank | starting at 221bp after the A of the ATG start codon, 845bp of nifL have been deleted and replaced with the CI019 Prm1.2 promoter sequence; 500bp flanking the nifL gene upstream and downstream are included |
| 17-724 | 19-804 | SEQ ID NO 223 | glnEΔAR-2 | glnE gene with 1650bp immediately downstream of the ATG start codon deleted, resulting in a truncated glnE protein lacking the adenylyl-removing (AR) domain |
| 17-724 | 19-804 | SEQ ID NO 224 | glnEΔAR-2 with 500bp flank | glnE gene with 1650bp immediately downstream of the ATG start codon deleted, resulting in a truncated glnE protein lacking the adenylyl-removing (AR) domain; 500bp flanking the glnE gene upstream and downstream are included |
| 19-590 | 19-806 | SEQ ID NO 225 | ΔnifL::Prm3.1 | starting at 221bp after the A of the ATG start codon, 845bp of nifL have been deleted and replaced with the CI019 Prm3.1 promoter sequence |
| 19-590 | 19-806 | SEQ ID NO 226 | ΔnifL::Prm3.1 with 500bp flank | starting at 221bp after the A of the ATG start codon, 845bp of nifL have been deleted and replaced with the CI019 Prm3.1 promoter sequence; 500bp flanking the nifL gene upstream and downstream are included |
| 19-590 | 19-806 | SEQ ID NO 227 | glnEΔAR-2 | glnE gene with 1650bp immediately downstream of the ATG start codon deleted, resulting in a truncated glnE protein lacking the adenylyl-removing (AR) domain |
| 19-590 | 19-806 | SEQ ID NO 228 | glnEΔAR-2 with 500bp flank | glnE gene with 1650bp immediately downstream of the ATG start codon deleted, resulting in a truncated glnE protein lacking the adenylyl-removing (AR) domain; 500bp flanking the glnE gene upstream and downstream are included |
| none | 63-1146 | SEQ ID NO 229 | ΔnifL::PinfC | starting at 24bp after the A of the ATG start codon, 1375bp of nifL have been deleted and replaced with the 63 PinfC promoter sequence |
| none | 63-1146 | SEQ ID NO 230 | ΔnifL::PinfC with 500bp flank | starting at 24bp after the A of the ATG start codon, 1375bp of nifL have been deleted and replaced with the 63 PinfC promoter sequence; 500bp flanking the nifL gene upstream and downstream are included |
| CM015; PBC6.15 | 6-397 | SEQ ID NO 231 | ΔnifL::Prm5 | starting at 31bp after the A of the ATG start codon, 1375bp of nifL have been deleted and replaced with the CI006 Prm5 promoter sequence |
| CM015; PBC6.15 | 6-397 | SEQ ID NO 232 | ΔnifL::Prm5 with 500bp flank | starting at 31bp after the A of the ATG start codon, 1375bp of nifL have been deleted and replaced with the CI006 Prm5 promoter sequence; 500bp flanking the nifL gene upstream and downstream are included |
| CM014 | 6-400 | SEQ ID NO 233 | ΔnifL::Prm1 | starting at 31bp after the A of the ATG start codon, 1375bp of nifL have been deleted and replaced with the CI006 Prm1 promoter sequence |
| CM014 | 6-400 | SEQ ID NO 234 | ΔnifL::Prm1 with 500bp flank | starting at 31bp after the A of the ATG start codon, 1375bp of nifL have been deleted and replaced with the CI006 Prm1 promoter sequence; 500bp flanking the nifL gene upstream and downstream are included |
| CM037; PBC6.37 | 6-403 | SEQ ID NO 235 | ΔnifL::Prm 1 | starting at 3 1bp after the A of the ATG start codon, 1375bp of nifL have been deleted and replaced with the CI006 Prm 1 promoter sequence |
| CM037; PBC6.38 | 6-403 | SEQ ID NO 236 | ΔnifL::Prm1 with 500bp flank | starting at 31bp after the A of the ATG start codon, 1375bp of nifL have been deleted and replaced with the CI006 Prm1 promoter sequence; 500bp flanking the nifL gene upstream and downstream are included |
| CM037; PBC6.39 | 6-403 | SEQ ID NO 237 | glnEΔAR-2 | glnE gene with 1644bp immediately downstream of the ATG start codon deleted, resulting in a truncated glnE protein lacking the adenylyl-removing (AR) domain |
| CM037; PBC6.40 | 6-403 | SEQ ID NO 238 | glnEΔAR-2 with 500bp flank | glnE gene with 1644bp immediately downstream of the ATG start codon deleted, resulting in a truncated glnE protein lacking the adenylyl-removing (AR) domain; 500bp flanking the glnE gene upstream and downstream are included |
| CM038; PBC6.38 | 6-404 | SEQ ID NO 239 | glnEΔAR-1 | glnE gene with 1287bp immediately downstream of the ATG start codon deleted, resulting in a truncated glnE protein lacking the adenylyl-removing (AR) domain |
| CM038; PBC6.38 | 6-404 | SEQ ID NO 240 | ΔnifL::Prml | starting at 3 1bp after the A of the ATG start codon, 1375bp of nifL have been deleted and replaced with the CI006 Prm1 promoter sequence |
| CM038; PBC6.38 | 6-404 | SEQ ID NO 241 | ΔnifL::Prm1 with 500bp flank | starting at 31bp after the A of the ATG start codon, 1375bp of nifL have been deleted and replaced with the C1006 Prm1 promoter sequence; 500bp flanking the nifL gene upstream and downstream are included |
| CM038; PBC6.38 | 6-404 | SEQ ID NO 242 | glnEΔAR-1 with 500bp flank | glnE gene with 1287bp immediately downstream of the ATG start codon deleted, resulting in a truncated glnE protein lacking the adenylyl-removing (AR) domain; 500bp flanking the glnE gene upstream and downstream are included |
| CM029; PBC6.29 | 6-412 | SEQ ID NO 243 | glnEΔAR-1 | glnE gene with 1287bp immediately downstream of the ATG start codon deleted, resulting in a truncated glnE protein lacking the adenylyl-removing (AR) domain |
| CM029; PBC6.29 | 6-412 | SEQ ID NO 244 | glnEΔAR-1 with 500bp flank | glnE gene with 1287bp immediately downstream of the ATG start codon deleted, resulting in a truncated glnE protein lacking the adenylyl-removing (AR) domain; 500bp flanking the glnE gene upstream and downstream are included |
| CM029; PBC6.29 | 6-412 | SEQ ID NO 245 | ΔnifL: :Prm5 | starting at 3 1bp after the A of the ATG start codon, 1375bp of nifL have been deleted and replaced with the CI006 Prm5 promoter sequence |
| CM029; PBC6.29 | 6-412 | SEQ ID NO 246 | ΔnifL::Prm5 with 500bp flank | starting at 31bp after the A of the ATG start codon, 1375bp of nifL have been deleted and replaced with the CI006 Prm5 promoter sequence; 500bp flanking the nifL gene upstream and downstream are included |
| CM093; PBC6.93 | 6-848 | SEQ ID NO 247 | ΔnifL::Prm1 | starting at 31bp after the A of the ATG start codon, 1375bp of nifL have been deleted and replaced with the CI006 Prm1 promoter sequence |
| CM093; PBC6.93 | 6-848 | SEQ ID NO 248 | ΔnifL::Prm1 with 500bp flank | starting at 31bp after the A of the ATG start codon, 1375bp of nifL have been deleted and replaced with the CI006 Prm1 promoter sequence; 500bp flanking the nifL gene upstream and downstream are included |
| CM093; PBC6.93 | 6-848 | SEQ ID NO 249 | glnEΔAR-2 | glnE gene with 1644bp immediately downstream of the ATG start codon deleted, resulting in a truncated glnE protein lacking the adenylyl-removing (AR) domain |
| CM093; PBC6.93 | 6-848 | SEQ ID NO 250 | glnEAAR-2 with 500bp flank | glnE gene with 1644bp immediately downstream of the ATG start codon deleted, resulting in a truncated glnE protein lacking the adenylyl-removing (AR) domain; 500bp flanking the glnE gene upstream and downstream are included |
| CM093; PBC6.93 | 6-848 | SEQ ID NO 251 | ΔamtB | First 1088bp of amtB gene and 4bp upstream of start codon deleted; 199bp of gene remaining lacks a start codon; no amtB protein is translated |
| CM093; PBC6.93 | 6-848 | SEQ ID NO 252 | ΔamtB with 500bp flank | First 1088bp of amtB gene and 4bp upstream of start codon deleted; 199bp of gene remaining lacks a start codon; no amtB protein is translated |
| CM094; PBC6.94 | 6-881 | SEQ ID NO 253 | glnEΔAR-1 | glnE gene with 1287bp immediately downstream of the ATG start codon deleted, resulting in a truncated glnE protein lacking the adenylyl-removing (AR) domain |
| CM094; PBC6.94 | 6-881 | SEQ ID NO 254 | glnEΔAR-1 with 500bp flank | glnE gene with 1287bp immediately downstream of the ATG start codon deleted, resulting in a truncated glnE protein lacking the adenylyl-removing (AR) domain; 500bp flanking the glnE gene upstream and downstream are included |
| CM094; PBC6.94 | 6-881 | SEQ ID NO 255 | ΔnifL::Prm 1 | starting at 3 1bp after the A of the ATG start codon, 1375bp of nifL have been deleted and replaced with the CI006 Prm1 promoter sequence |
| CM094; PBC6.94 | 6-881 | SEQ ID NO 256 | ΔnifL::Prm1 with 500bp flank | starting at 31bp after the A of the ATG start codon, 1375bp of nifL have been deleted and replaced with the CI006 Prm1 promoter sequence; 500bp flanking the nifL gene upstream and downstream are included |
| CM094; PBC6.94 | 6-881 | SEQ ID NO 257 | ΔamtB | First 1088bp of amtB gene and 4bp upstream of start codon deleted; 1 99bp of gene remaining lacks a start codon; no amtB protein is translated |
| CM094; PBC6.94 | 6-881 | SEQ ID NO 258 | ΔamtB with 500bp flank | First 1088bp of amtb gene and 4bp upstream of start codon deleted; 199bp of gene remaining lacks a start codon; no amtB protein is translated |
| none | 910-1246 | SEQ ID NO 259 | ΔnifL::PinfC | starting at 20bp after the A of the ATG start codon, 1379bp of nifL have been deleted and replaced with the 910 PinfC promoter sequence |
| none | 910-1246 | SEQ ID NO 260 | ΔnifL::PinfC with 500bp flank | starting at 20bp after the A of the ATG start codon, 1379bp of nifL have been deleted and replaced with the 910 PinfC promoter sequence; 500bp flanking the nifL gene upstream and downstream are included |
| PBC6.1, 6, CI6 | CI006 | SEQ ID NO 261 | 16S-1 | 1 of 3 unique 16S rDNA genes in the CI006 genome |
| PBC6.1, 6, CI6 | CI006 | SEQ ID NO 262 | 16S-2 | 2 of 3 unique 16S rDNA genes in the CI006 genome |
| PBC6.1, 6. CI6 | CI006 | SEQ ID NO 263 | nifH | N/A |
| PBC6.1, 6, CI6 | CI006 | SEQ ID NO 264 | nifD2 | 2 of 2 unique genes annotated as nifD in CI006 genome |
| PBC6.1, 6, CI6 | CI006 | SEQ ID NO 265 | nifK2 | 2 of 2 unique genes annotated as nifK in CI006 genome |
| PBC6.1, 6, C16 | CI006 | SEQ ID NO 266 | nifL | N/A |
| PBC6.1, 6, CI6 | CI006 | SEQ ID NO 267 | nifA | N/A |
| PBC6.1, 6, CI6 | CI006 | SEQ ID NO 268 | glnE | N/A |
| PBC6.1, 6, CI6 | CI006 | SEQ ID NO 269 | 16S-3 | 3 of 3 unique 16S rDNA genes in the CI006 genome |
| PBC6.1, 6, CI6 | CI006 | SEQ ID NO 270 | nifD1 | 1 of 2 unique genes annotated as nifD in CI006 genome |
| PBC6.1, 6, CI6 | CI006 | SEQ ID NO 271 | nifK1 | 1 of 2 unique genes annotated as nifK in CI006 genome |
| PBC6.1, 6, CI6 | CI006 | SEQ ID NO 272 | amtB | N/A |
| PBC6.1, 6, CI6 | CI006 | SEQ ID NO 273 | Prm1 | 348bp includes the 319bp immediately upstream of the ATG start codon of the lpp gene and the first 29bp of the lpp gene |
| PBC6.1, 6, CI6 | CI006 | SEQ ID NO 274 | Prm5 | 313bp starting at 432bp upstream of the ATG start codon of the ompX gene and ending 119bp upstream of the ATG start codon of the ompX gene |
| 19, C119 | CI019 | SEQ ID NO 275 | nifL | N/A |
| 19, CI199 | CI019 | SEQ ID NO 276 | nifA | N/A |
| 19, C119 | CI019 | SEQ ID NO 277 | 16S-1 | 1 of 7 unique 16S rDNA genes in the CI0 19 genome |
| 19, CI19 | CI019 | SEQ ID NO 278 | 16S-2 | 2 of 7 unique 16S rDNA genes in the CI019 genome |
| 19, C119 | CI0119 | SEQ ID NO 279 | 16S-3 | 3 of 7 unique 16S rDNA genes in the CI019 genome |
| 19, CI19 | CI019 | SEQ ID NO 280 | 16S-4 | 4 of 7 unique 16S rDNA genes in the CI019 genome |
| 19, CI19 | CI019 | SEQ ID NO 281 | 16S-5 | 5 of 7 unique 16S rDNA genes in the CI0 19 genome |
| 19, CI19 | CI019 | SEQ ID NO 282 | 16S-6 | 6 of 7 unique 16S rDNA genes in the CI019 genome |
| 19, CI19 | CI019 | SEQ ID NO 283 | 16S-7 | 7 of 7 unique 16S rDNA genes in the CI019 genome |
| 19, CI19 | CI019 | SEQ ID NO 284 | nifH1 | 1 of 2 unique genes annotated as nifH in CI019 genome |
| 19, CI19 | CI019 | SEQ ID NO 285 | nifH2 | 2 of 2 unique genes annotated as nifH in CI019 genome |
| 19, CI19 | CI019 | SEQ ID NO 286 | nifD1 | 1 of 2 unique genes annotated as nifD in CI019 genome |
| 19, CI19 | CI019 | SEQ ID NO 287 | nifD2 | 2 of 2 unique genes annotated as nifD in CI019 genome |
| 19, CI19 | CI019 | SEQ ID NO 288 | nifK1 | 1 of 2 unique genes annotated as nifK in CI019 genome |
| 19, CI19 | CI019 | SEQ ID NO 289 | nifK2 | 2 of 2 unique genes annotated as nifK in CI019 genome |
| 19, CI199 | CI019 | SEQ ID NO 290 | glnE | N/A |
| 19, CI19 | CI019 | SEQ ID NO 291 | Prm4 | 449bp immediately upstream of the ATG of the dscC 2 gene |
| 19, CI19 | CI019 | SEQ ID NO 292 | Prm1.2 | 500bp immediately upstream of the TTG start codon of the infC gene |
| 19, CI19 | CI019 | SEQ ID NO 293 | Prm3.1 | 170 bp immediately upstream of the ATG start codon of the rplN gene |
| 19, Cl20 | CI020 | SEQ ID NO 294 | Prm6.1 | 142bp immediately upstream of the ATG of a highly-expressed hypothetical protein (annotated as PROKKA_00662 in CI019 assembly 82) |
| 19, CI21 | CI021 | SEQ ID NO 295 | Prm7.1 | 293bp immediately upstream of the ATG of the 1pp gene |
| 19-375, 19-417, CM067 | CM67 | SEQ ID NO 296 | glnEΔAR-2 | glnE gene with 1650bp immediately downstream of the ATG start codon deleted, resulting in a truncated glnE protein lacking the adenylyl-removing (AR) domain |
| 19-375, 19-417, CM067 | CM67 | SEQ ID NO 297 | glnEAAR-2 with 500bp flank | glnE gene with 1650bp immediately downstream of the ATG start codon deleted, resulting in a truncated glnE protein lacking the adenylyl-removing (AR) domain; 500bp flanking the glnE gene upstream and downstream are included |
| 19-375, 19-417, CM067 | CM67 | SEQ ID NO 298 | ΔnifL::null-v1 | starting at 221bp after the A of the ATG start codon, 845bp of nifL have been deleted and replaced with the 31bp sequence “GGAGTCTGAACTCATCCTGCGA TGGGGGCTG” |
| 19-375, 19-417, CM067 | CM67 | SEQ ID NO 299 | ΔnifL::null-v1 with 500bp flank | starting at 221bp after the A of the ATG start codon, 845bp of nifL have been deleted and replaced with the 31bp sequence “GGAGTCTGAACTCATCCTGCGA TGGGGGCTG”; 500bp flanking the nifL gene upstream and downstream are included |
| 19-377, CM069 | CM69 | SEQ ID NO 300 | ΔnifL::null-v2 | starting at 221bp after the A of the ATG start codon, 845bp of nifL have been deleted and replaced with the 5bp sequence “TTAAA” |
| 19-377, CM069 | CM69 | SEQ ID NO 301 | ΔnifL::null-v2 with 500bp flank | starting at 221bp after the A of the ATG start codon, 845bp of nifL have been deleted and replaced with the 5bp sequence “TTAAA”; 500bp flanking the nifL gene upstream and downstream are included |
| 19-389, 19-418, CM081 | CM81 | SEQ ID NO 302 | ΔnifL::Prm4 | starting at 221bp after the A of the ATG start codon, 845bp of nifL have been deleted and replaced with the CI19 Prm4 sequence |
| 19-389, 19-418, CM081 | CM81 | SEQ ID NO 303 | ΔnifL::Prm4 with 500bp flank | starting at 221bp after the A of the ATG start codon, 845bp of nifL have been deleted and replaced with the CI19 Prm4 sequence; 500bp flanking the nifL gene upstream and downstream are included |
| none | 137-3890 | SEQ ID NO 458 | ΔnifL-Prm1.2 | starting at 24bp after the A of the ATG start codon, 1372bp of nifL have been deleted and replaced with the 137 Prm1.2 promoter sequence |
| none | 137-3890 | SEQ ID NO 459 | ΔnifL-Prm1.2 with 500bp flank | starting at 24bp after the A of the ATG start codon, 1372bp of nifL have been deleted and replaced with the 137 Prm1.2 promoter sequence; 500bp flanking the nifL gene upstream and downstream are included |
| none | 137-3890 | SEQ ID NO 460 | glnE_KO2 | glnE gene with 1290bp immediately downstream of the ATG start codon deleted, resulting in a truncated glnE protein lacking the adenylyl-removing (AR) domain |
| none | 137-3890 | SEQ ID NO 461 | glnE_KO2 with 500bp flank | glnE gene with 1290bp immediately downstream of the ATG start codon deleted, resulting in a truncated glnE protein lacking the adenylyl-removing (AR) domain; 500bp flanking the glnE gene upstream and downstream are included |
| none | 137-3890 | SEQ ID NO 462 | NtrC_D54A | Deactivation of the phosphorylation site of the DNA-binding transcriptional regulator NrtC by swapping the 54th amino acid from aspartate to alanine (D to A) by changing the GAT codon to GCT. Disables the ability of NtrC to be phosphorylated. |
| none | 137-3890 | SEQ ID NO 463 | NtrC_D54A with flanking sequences | Deactivation of the phosphorylation site of the DNA-binding transcriptional regulator NrtC by swapping the 54th amino acid from aspartate to alanine (D to A) by changing the GAT codon to GCT. Disables the ability of NtrC to be phosphorylated. 693bp upstream and 549bp downstream NtrC sequences flanking NtrCD54A mutation are included. |
| none | 137-3896 | SEQ ID NO 464 | ΔnifL::PinfC | Deletion of the nifL gene from 20bp after the ATG (start) to 87bp before the TGA (stop) of the gene. A 500bp fragment from the region upstream of the infC gene was inserted (PinfC) upstream of nifA replacing the deleted portion. |
| none | 137-3896 | SEQ ID NO 465 | AnifL::PinfC with flanking sequences | Deletion of the nifL gene from 20bp after the ATG (start) to 87bp before the TGA (stop) of the gene. A 500bp fragment from the region upstream of the infC gene was inserted (PinfC) upstream of nifA replacing the deleted portion; 332bp upstream and 324bp downstream flanking the nifL gene are included. |
| none | 137-3896 | SEQ ID NO 466 | glnD_UTase_Deacti vation | Deactivation of the uridylyltransferase (UT) domain of the bifunctional uridylyltransferase/uridylyl-removing enzyme, glnD, by mutating amino acid residues 90 and 91 from GG to DV as well as residue 104 from D to A. |
| none | 137-3896 | SEQ ID NO 467 | glnD_UTase_Deacti vation with flanking sequences | Deactivation of the uridylyltransferase (UT) domain of the bifunctional uridylyltransferase/uridylyl-removing enzyme, glnD, by mutating amino acid residues 90 and 91 from GG to DV as well as residue 104 from D to A; 450bp flanking the mutated sites upstream and downstream are included. |
| none | 137-3896 | SEQ ID NO 468 | NC-nifA_copy::Prml.2 | Insertion of a copy of the nifA gene into a noncoding region of 137. This copy is being driven by a 400bp promoter (Prm1.2) derived from a region upstream of the cspE gene. |
| none | 137-3896 | SEQ ID NO 469 | NC-nifA_copy::Prm1.2 with flanking sequences | Insertion of a copy of the nifA gene into a noncoding region of 137. This copy is being driven by a 400bp promoter (Prm1.2) derived from a region upstream of the cspE gene; 2000bp flanking the insertion site upstream and downstream are included. |

### EXAMPLES

### Example 1: Ecological Sidedressing and Weatherproof Nitrogen

Sustainable production of grains such as corn, wheat, and rice require the application of some source of nitrogen. Growers apply nitrogen that plants can use in a number of forms. In geographies where livestock production is intense, livestock manure can meet a significant portion of the nitrogen needs of a corn crop. Where no organic form of nitrogen is available, commercial nitrogen fertilizers either in the form of a gas held under pressure as a liquid (NH3) a dry formulation such as ammonium nitrate or urea, or in liquid formulations such as combinations of urea and ammonia nitrate (UAN).

The point in time when nitrogen is applied to corn depends upon a number of factors. The first of these may be local or state regulations. Other factors that may affect when a grower chooses to apply nitrogen would be field-working conditions in the fall (still a popular application timing for many geographies) due to uncertainty around cropping plans, Spring weather, and planting conditions and the size of the operation.

Growers may apply nitrogen in the fall, after the previous crop is removed. This application timing, while popular, is under attack by regulatory agencies who are seeking to limit either the number of pounds that can be applied in the Fall or the Fall application entirely. If no Fall application occurs, then growers will usually apply nitrogen prior to planting the corn crop, after crop emergence, or a combination of the two, which is referred to as a split application.

In any of the aforementioned nitrogen delivery regimes, the second application of nitrogen, which normally occurs at the V4-V6 stage, is referred to as a sidedress application. The sidedress application of nitrogen is often applied between the rows.

Due to the instability of nitrogen molecules once they are in the soil, research has demonstrated that if a grower can apply the nitrogen as close to when the corn crop needs the nitrogen, there are significant benefits for the crop as well as for the environment. The nitrogen use efficiency increases, meaning it takes less pounds of nitrogen to produce a bushel.

Sidedressing is not without risks. The ability to get across all of a grower’s acres in a timely manner is not ensured. These risks increase as the size of the operation increases and as potential changes to the climate make the number of days suitable for fieldwork less predictable.

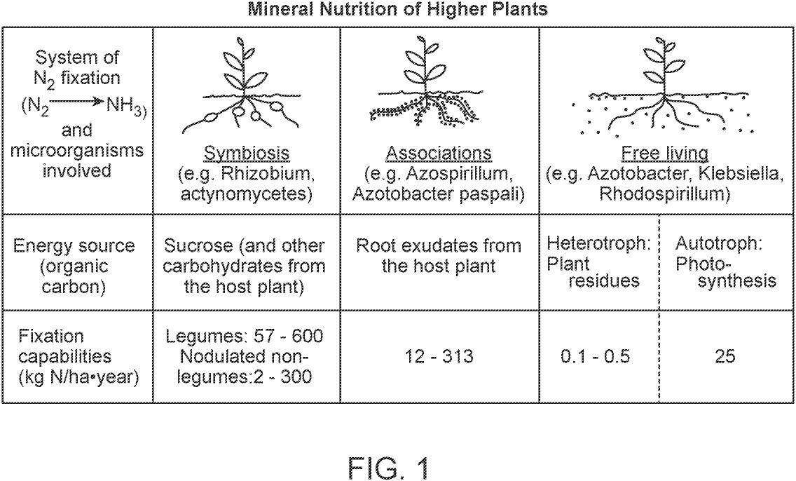

An alternative to the use of commercial fertilizer for legumes (primarily soybeans) has been biological nitrogen fixing (BNF) systems, which exist in nature. These systems fall into one of three types and differ in their use of substrate and efficiency. See FIG. 1.

An example of where the majority of the nitrogen needs of the crop are met through a symbiotic relationship with the plant would be that of soybeans or alfalfa. They are capable of converting almost enough molecular nitrogen (N₂) to meet the nitrogen needs of the crop. In the case of soybeans, many farmers apply Rhizobium at the time of planting, but some Rhizobium are ubiquitous in most soils and populations are able to survive in the soil from year to year.

The ability to produce a microbe that would be able to convert N₂ to NH₃ through root association in cereals such as corn, rice, or wheat would be revolutionary and the equivalent of BNF in soybeans. It could also replace sidedressing since both practices would allow for the timely delivery of nitrogen to the growing plant in season. BNF for cereals would also allow growers to reduce the risks associated with sidedressing. These risks include reduced yields due to untimely applications, variable in-season cost of nitrogen, the cost of application, and consistency of nitrogen availability in years when environmental conditions are conducive to loss through de-nitrification or leaching. BNF for cereals would also create value through ease of use and reducing passes over the field for specific nitrogen applications.

As can be seen from the below Table 5, Fall and Spring nitrogen application strategies always use sidedress. The split application also features sidedressing. The state of the art is such that sidedressing is an energy intensive mechanical process that is applied by a tractor that compacts the soil. Often at stage V4-V6, additional nitrogen is applied as sidedressing.

The disclosed remodeled nitrogen fixing bacteria are able to eliminate the practice of sidedressing, as these bacteria live in intimate association with the plant’s root system and “spoonfeed” the plant nitrogen.

TABLE 5

Comparison of Current Nitrogen Application Timing Practices and Proposed Microbial Introduction Practices

| | | |
|---|---|---|
| Nitrogen Application Timing Practices | Proposed Microbial Introduction Practices | Benefits of the Proposed Microbial Introduction Over Previous Nitrogen Application Timing |
| Fall application - 100% of crop needs | At planting either as seed treatment or in furrow application | Potential to reduce rates applied in the fall |
| | | No need to apply supplemental applications in crop if spring weather conditions are conducive to nitrogen loss More consistent yields across the geography due to supplemental nitrogen being available in soil types where conditions for nitrogen loss are higher than in other parts of the field |
| Early spring applications -100% of crop needs | At planting either as a seed treatment or in furrow application | No need to apply supplemental applications in crop if weather conditions are conducive to nitrogen loss after application More consistent yields across the geography due to supplemental nitrogen being available in soil types where conditions for nitrogen loss are higher than in other parts of the field |
| Planned Split applications 150 1b followed by 30 lbs | At planting either as a seed treatment or in furrow application | Reduces the needs for the second application Ensures that split application is applied to all acres Ensures that the application is applied in a timely manner to prevent yield loss Ensures that the application is done in a timely manner as to prevent damage to the crop through the pruning of roots More consistent yields across the geography due to |
| | | supplemental nitrogen being available in soil types where conditions for nitrogen loss are higher than in other parts of the field |

Thus, as can be seen in Table 5, the present disclosure provides an alternative to traditional synthetic fertilizer sidedressing, by allowing a farmer to utilize an “ecological sidedressing” comprised of non-intergeneric remodeled bacteria that are capable of fixing atmospheric nitrogen and delivering such to the corn plant throughout the corn’s growth cycle.

### Example 2: Remodeling Microbial Systems for Temporally and Spatially Targeted Dynamic Nitrogen Delivery

The microbes of the disclosure are engineered with one or more of the following features, in order to develop non-intergeneric remodeled microbes that are capable of colonizing corn and supplying fixed nitrogen to the corn, at physiologically relevant periods of the corn’s life cycle.

These genetic modifications provide the building blocks of a Guided Microbial Remodeling (GMR) campaign, which will be elaborated upon below.

Feature: Nitrogenase Expression - nifL deletion and promoter insertion upstream of nifA.

NifA activates the nif gene complex and drives nitrogen fixation when there is insufficient fixed nitrogen available to the microbe. NifL inhibits NifAwhen there is sufficient fixed N available to the microbe. The nifL and nifA genes are present in an operon and are driven by the same promoter upstream of nifL, which is activated in conditions of nitrogen insufficiency and repressed in conditions of nitrogen sufficiency (FIG. 1, Dixon and Kahn 2004). In this feature, we have deleted most of the nifL coding sequence and replaced it with a constitutive promoter naturally present elsewhere in the genome of the wild-type strain which we have observed is highly expressed in nitrogen-replete conditions. This allows NifA to be both expressed and active in nitrogen-replete conditions, such as a fertilized field.

Feature: Nitrogenase Expression - Promoter swap of the rpoN gene to increase availability of sigma factor 54

Sigma factors are required for initiation of transcription of prokaryotic genes, and sometimes specific sigma factors initiate the transcription of a set of genes in a common regulatory network. Sigma 54 (σ⁵⁴), encoded by the gene rpoN, is responsible for transcription of many genes involved in nitrogen metabolism, including the nif cluster and nitrogen assimilation genes (Klipp et al. 2005, Genetics and Regulation of Nitrogen Fixation in Free-Living Bacteria, Kluwer Academic Publishers (Vol. 2). doi.org/10.1007/1-4020-2179-8). In strains where nifA is controlled by a strong promoter active in nitrogen replete conditions, the availability of σ⁵⁴ to initiate transcription of the nif genes may become limiting. In this feature, the promoter of the rpoN gene has been disrupted by deleting the intergenic sequence immediately upstream of the gene. The deleted sequence was replaced by a different promoter naturally present elsewhere in the genome of the wild-type strain, which we have observed is highly expressed in nitrogen-replete conditions. This results in increased expression of σ⁵⁴ which relieves any limitation on transcription initiation in strains highly expressing nifA.

### Feature: Nitrogen Assimilation - Deletion of the Adenylyl-Removing Domain of GlnE

Fixed nitrogen is primarily assimilated by the microbe by the glutamine synthetase/glutamine oxoglutarate aminotransferase (GS-GOGAT) pathway. The resulting glutamine and glutamate pools in the cell control nitrogen metabolism, with glutamate serving as the main nitrogen pool for biosynthesis and glutamine serving as the signaling molecule for nitrogen status. The glnE gene encodes an enzyme, known as glutamine synthetase adenylyl transferase or glutamine-ammonia-ligase adenylyl transferase, that regulates the activity of glutamine synthetase (GS), in response to intracellular levels of glutamine. The GlnE protein consists of two domains with independent and distinct enzymatic activities: an adenylyltransferase (ATase) domain, which covalently modifies the GS protein with an adenylyl group, thus reducing GS activity; and an adenylyl-removing (AR) domain, which removes the adenylyl group from GS, thus increasing its activity. Clancy et al. (2007) showed that truncation of the Escherichia coli K12 GlnE protein to remove the AR domain lead to expression of a protein that retains ATase activity. In this feature, we have deleted the N-terminal AR domain of GlnE, resulting in a strain lacking the AR activity, but functionally expressing the ATase domain. This leads to constitutively adenylated GS with attenuated activity, causing a reduction in assimilation of ammonium and excretion of ammonium out of the cell.

### Feature: Nitrogen Assimilation - Decrease Transcription and/or Translation Rates of Gene Encoding GS

The glnA gene, which encodes the GS enzyme, is controlled by a promoter which is activated under nitrogen depletion, and repressed under nitrogen replete conditions (Van Heeswijk et al. 2013). In this feature, the amount of GS enzyme in the cell has been decreased in at least one of two ways (or a combination of the following two ways into one cell). First, the “A” of the ATG start codon of the glnA gene, which encodes glutamine synthetase (GS), has been changed to “G”. The rest of the glnA gene and GS protein sequence remains unaltered. The resulting GTG start codon is hypothesized to result in a decreased translation initiation rate of the glnA transcript, leading to a decrease in the intracellular level of GS. Second, the promoter upstream of the glnA gene has been disrupted by deleting the intergenic sequence immediately upstream of the gene. The deleted sequence was replaced by the promoter of the glnD, glnE or glnB genes, which are expressed constitutively at a very low level regardless of nitrogen status (Van Heeswijk et al 2013). This leads to a decrease in glnA transcription levels s and therefore a decrease in GS levels in the cell. As aforementioned, the previous two scenarios (alteration of start codon and promoter disruption) can be combined into a host. The decreased GS activity in the cell leads to a decrease in the bacterial assimilation of the ammonium produced by nitrogen fixation, resulting in excretion of ammonium outside of the bacterial cell, making nitrogen more available for plant uptake (Ortiz-Marquez, J. C. F., Do Nascimento, M., & Curatti, L. (2014) “Metabolic engineering of ammonium release for nitrogen-fixing multispecies microbial cell-factories,” Metabolic Engineering, 23, 1-11. doi.org/10.1016/j.ymben.2014.03.002).

### Feature: Nitrogen Assimilation - Promoter Swap of the glsA2 Gene to Increase Glutaminase Activity

Glutaminase enzymes catalyze the release of ammonium from glutamine and may play an important role in controlling the intracellular glutamine pool (Van Heeswijk et al. 2012). In this feature, the glsA2 gene encoding glutaminase has been upregulated by deleting a sequence immediately upstream of the gene and replacing it with different promoter naturally present elsewhere in the genome which is highly expressed in nitrogen-replete conditions. This results in increased expression of glutaminase enzyme in the cell, leading to release of ammonium from the glutamine pool and therefore increased excretion of ammonium out of the cell.

### Feature: Ammonium Excretion - Amtb Deletion

The amtB gene encodes a transport protein that functions to import ammonium from the extracellular space into the cell interior. It is believed that in nitrogen-fixing bacteria, the AmtB protein functions to ensure that any ammonium that passively diffuses out of the cell during nitrogen fixation is imported back into the cell, thus preventing loss of fixed nitrogen (Zhang et al. 2012). In this feature, the amtB coding sequence has been deleted, leading to net diffusion of ammonium out of the cell and thus an increase in ammonium excretion (Barney et al. 2015). The amtB promoter has been left intact.

### Feature: Robustness and Colonization - Promoter Swap of BcsII and BcsIII Operons to Increase Bacterial Cellulose Production

Bacterial cellulose biosynthesis is an important factor for both attachment to the root and biofilm formation on root surfaces (Rodriguez-Navarro et al. 2007). The bcsII and bcsIII operons each encode a set of genes involved in bacterial cellulose biosynthesis (Ji et al. 2016). In this feature, the native promoter of the bcsII operon has been disrupted by deleting the intergenic region upstream of the first gene in the operon and replacing it with a different promoter naturally present elsewhere in the genome of the wild-type strain which we have observed is highly expressed in nitrogen-replete conditions. This results in increased expression of the bcsII operon in a fertilized-field environment, which leads to an increase in bacterial cellulose production and thus attachment to corn roots.

### Feature: Promoter Swap of PehA Operon to Increase Polygalacturonase Production

Polygalacturonases are implicated as important factors for colonization of plant roots by non-nodule-forming bacteria (Compant, S., Clément, C., & Sessitsch, A. (2010), “Plant growth-promoting bacteria in the rhizo- and endosphere of plants: Their role, colonization, mechanisms involved and prospects for utilization,” Soil Biology and Biochemistry, 42(5), 669-678. doi.org/10.1016/j.soilbio.2009.11.024.)

The pehA gene encodes a polygalacturonase in an operon with two uncharacterized protein coding regions, with the pehA at the downstream end of the operon. In this feature, the promoter of the pehA operon has been disrupted by deleting a sequence immediately upstream of the first gene in the operon. The deleted sequence was replaced by a different promoter naturally present elsewhere in the genome of the wild-type strain, which we have observed is highly expressed in nitrogen-replete conditions. This results in increased expression of the PehA polygalacturonase protein in a fertilized-field environment, which leads to enhanced colonization of corn roots by the microbe.

### Feature: Robustness and Colonization - Promoter Swap of the fhaB Gene to Increase Expression of Adhesins

Bacterial surface adhesins, such as agglutinins, have been implicated in attachment, colony and biofilm formation on plant roots (Danhorn, T., & Fuqua, C. (2007), “Biofilm formation by plant-associated bacteria. Annual Review of Microbiology, 61, 401-422. doi.org/10.1146/annurev.micro.61.080706.093316).

The fhaB gene encodes a filamentous hemagglutinin protein. In this feature, the promoter of the fhaB gene has been disrupted by deleting the intergenic sequence immediately upstream of the gene. The deleted sequence was replaced by a different promoter naturally present elsewhere in the genome of the wild-type strain, which we have observed is highly expressed in nitrogen-replete conditions. This results in increased expression of the hemagglutinin protein, leading to increased root attachment and colonization.

### Feature: Robustness and Colonization - Promoter Swap of the DctA Gene to Increase Expression of Organic Acid Transporters

For successful colonization of the rhizosphere, a bacterium must have the ability to utilize carbon sources found in root exudates, such as organic acids. The gene dctA encodes an organic acid transporter that has been shown to be necessary for effective colonization in rhizosphere bacteria and repressed in response to exogenous nitrogen (Nam, H. S., Anderson, A. J, Yang, K. Y., Cho, B. H., & Kim, Y. C. (2006), “The dctA gene of Pseudomonas chlororaphis O6 is under RpoN control and is required for effective root colonization and induction of systemic resistance,” FEMS Microbiology Letters, 256(1), 98-104. doi.org/10.1111/1.1574-6968.2006.00092.x). In this feature, the promoter of the dctA gene has been disrupted by deleting the intergenic sequence immediately upstream of the gene. The deleted sequence was replaced by a different promoter naturally present elsewhere in the genome of the wild-type strain, which we have observed is highly expressed in the rhizosphere in nitrogen-replete conditions. This results in increased expression of the DctA transporter, enhanced utilization of root exudate carbon and thus improved robustness in fertilized-field conditions.

### Feature: Robustness and Colonization - Promoter Swap of the PhoB Gene to Promote Biofilm Formation

In rhizosphere bacteria, the PhoR-PhoB two-component system mediates a response to phosphorous limitation and has been linked to colony and biofilm formation on plant roots (Danhorn and Fuqua 2007). In this feature, the promoter of the phoB1 gene has been disrupted by deleting the intergenic sequence immediately upstream of the gene. The deleted sequence was replaced by a different promoter naturally present elsewhere in the genome of the wild-type strain, which we have observed is highly expressed in the rhizosphere in nitrogen-replete conditions. This results in increased expression of the PhoB component of the PhoR-PhoB system, leading to enhanced colony and biofilm formation on roots.

### Feature: Correlated Metabolic and Regulatory Networks - Altering Nitrogen Signaling to Influence Stress Response

GlnD is the central nitrogen-sensing enzyme in the cell. The GlnD protein consists of three domains: a uridylyl-transferase (UTase) domain, and (UR) uridylyl-removing domain, and a glutamine-binding ACT domain. In nitrogen-excess conditions, intracellular glutamine binds to the ACT domain of GlnD, causing the UTase domain to uridylylate the PII proteins GlnB and GlnK, causing a regulatory cascade upregulating genes involved in nitrogen fixation and assimilation. In nitrogen starvation conditions, glutamine is not available to bind to the ACT domain of GlnD, which causes the UR domain to de-uridylylate GlnK and GlnB, which causes repression of genes involved in nitrogen assimilation and repression. These PII regulatory cascades regulate several pathways, including nitrogen starvation stress responses, nitrogen assimilation and nitrogen fixation in diazotrophs (Dixon and Kahn 2004; van Heeswijk et al. 2013). In this feature, either the UTase domain, the UR domain, the ACT domain, or the entire gene encoding the GlnD protein has been modified in order to alter the transduction of nitrogen starvation signals which cause stress responses.

### Feature: Correlated Metabolic and Regulatory Networks - Deletion of the GlgA Glycogen Synthase Gene

Because nitrogen fixation is such an energy-intensive process, it is believed to be limited by the availability of ATP in the cell. It has therefore been hypothesized that diverting carbon away from energy storage pathways and towards oxidative phosphorylation could enhance nitrogen fixation in diazotrophs (Glick 2012). One study suggested that deletion of glgA gene, which encodes glycogen synthase, led to enhanced nitrogen fixation in legume-Rhizobia symbiosis (Marroquí et al. 2001). In this feature, the entire glgA. gene has been deleted in order to abolish glycogen synthesis. The deletion of the glgA gene leads to increased levels of nitrogen fixation in both nitrogen-starvation and nitrogen-replete conditions.

### GMR Campaign Utilizing Genetic Features

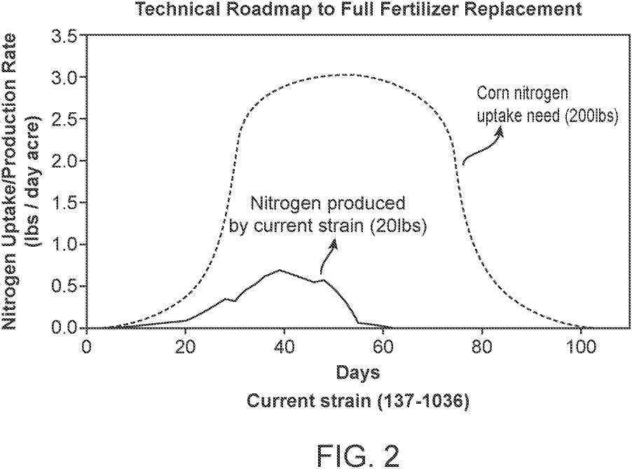

The microbes of the disclosure have been engineered to contain one or more of the aforementioned features. The overall goal of the GMR campaigns is to develop microbes that are capable of supplying all of the nitrogen needs of a corn plant throughout the entirety of a growing season. In FIG. 2, the inventors have calculated that in order for a nitrogen fixing microbe to supply a corn plant with all of its nitrogen needs over a growing season, and thus completely replace synthetic fertilizer, then the microbes (in the aggregate) need to produce about 200 pounds of nitrogen per acre. FIG. 2 also illustrates that strain PBC 137-1036 (i.e. the remodeled Klebsiella variicola) supplies about 20 pounds of nitrogen per acre.

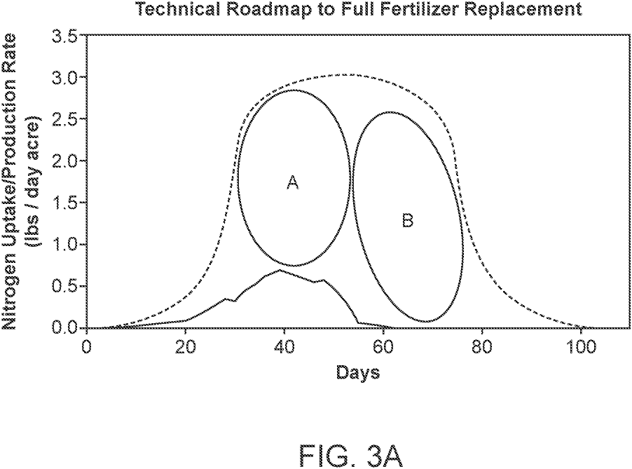

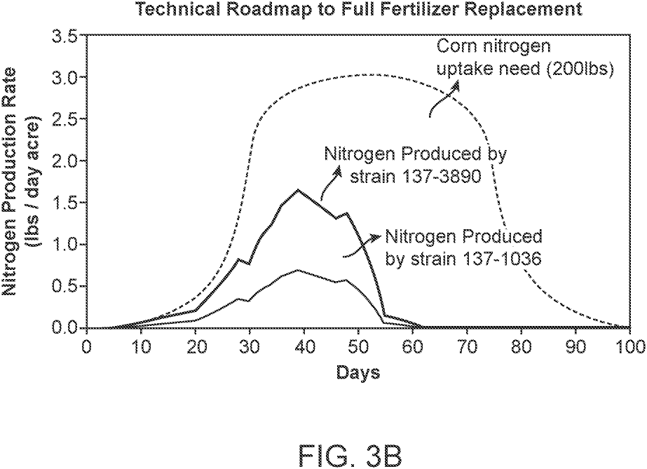

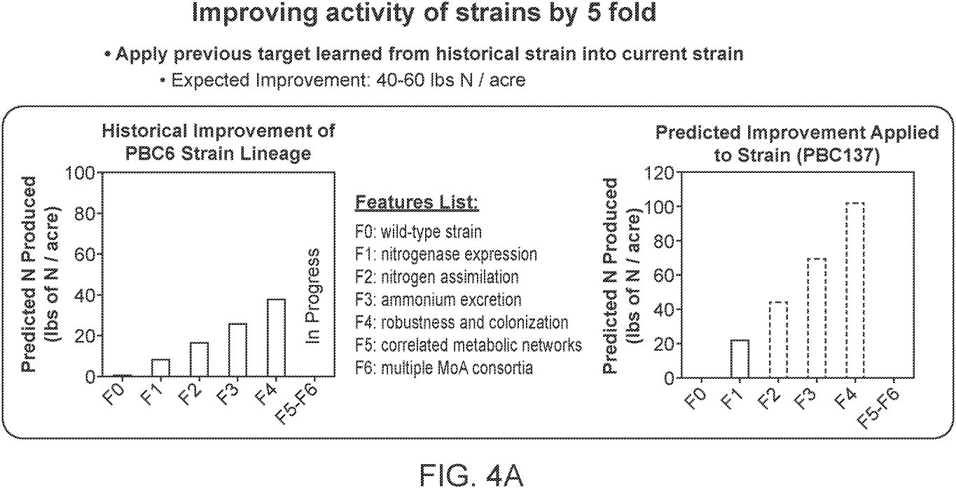

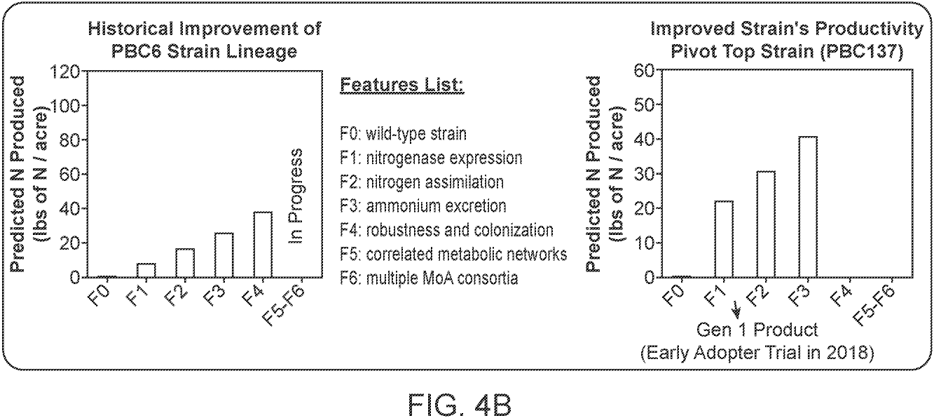

FIG. 3B shows the nitrogen produced by PBC 137-3890, a remodeled strain of Klebsiella variicola. FIG. 3A provides a scenario whereby fertilizer could be replaced by the remodeled microbes of the disclosure. As aforementioned in FIG. 2, the large dashed line is the nitrogen required by the corn (about 200 pounds per acre). The solid line, as already discussed, is the current nitrogen amount that can be supplied by the remodeled 137-1036 strain (about 20 pounds per acre). In the gray-shaded oval “A” scenario of FIG. 3A, the inventors expect to increase the activity of the 137-1036 strain by 5 fold (see FIGS. 4A-4B for GMR campaign strategy to achieve such). In the gray-shaded oval “B” scenario of FIG. 3A, the inventors expect to utilize a remodeled microbe with a particular colonization profile that is complementary to that of the 137-1036 strain, and which will supply nitrogen to the plant at later stages of the growth cycle. Since the filing of the provisional application, the inventors have been successful in improving the nitrogen production activity of the 137-1036 strain through the GMR campaign. Specifically, FIG. 3B shows the nitrogen production by the strain 137-3890, which is a further remodeled strain of 137-1036 obtained by employing the GMR campaign described in the application. As shown in FIG. 3B, the nitrogen production activity of 137-3890 is substantially improved compared to 137-1036.

FIG. 4B shows the predicted N produced (lbs of N per acre) after the features F2 and F3 were incorporated in the PBC137 (Klebsiella variicola).

In FIG. 4A, left panel, the discussed features (i.e. non-intergeneric genetic modifications) are illustrated with respect to a historical GMR campaign for PBC6.1 (Kosakonia sacchari). As can be seen in FIG. 4A, left panel, the predicted N produced (lbs of N per acre) increased with each additional feature engineered into the microbial strain.

In addition to the historical GMR campaign for PBC6.1 depicted in FIG. 4A, left panel, one can also see the GMR campaign being executed for the PBC137 (Klebsiella variicola), FIG. 4A, right panel. At the time the provisional application was filed, the nitrogenase expression feature (F1) was engineered into the host strain and features 2-6 were being executed. The expected contribution of each of these features to N produced (lbs of N per acre) is depicted in the dashed bar graphs in FIG. 4A. As can be seen in FIG. 3A, the gray-shaded oval scenario “A”, once the GMR campaign is completed in PBC137, it is anticipated that the non-intergeneric remodeled strain (in the aggregate, considering all microbes/colonized plants in an acre) will be capable of supplying nearly all of the nitrogen needs of a corn plant throughout the plant’s early growth cycle. Further, FIG. 5A depicts the same expectation, and maps the expected gains in nitrogen production to the applicable feature set at the time the provisional application was filed. Since the filing of the provisional application, the inventors have been working on engineering features F2-F6 into the host strain. At the time of filing the present application, the features F2 (nitrogen assimilation) and F3 (ammonium excretion) have been engineered into the PBC137 host strain. FIG. 4B, right panel, depicts the N produced by the remodeled strains upon incorporation of the features F1-F3. As can be seen from the right panel of FIG. 4B, the N produced (lbs of N per acre) increased with each additional feature engineered into the microbial strain. FIG. 5B depicts N produced as mmol of N/CFU per hour by the remodeled strains of PBC137 once the features F1 (nitrogenase expression), F2 (nitrogen assimilation), and F3 (ammonium excretion) were incorporated.

The mutations made to the PBC137 WT strain to incorporate the features F1-F3 are summarized in Table 6 below.

TABLE 6

List of isolated and derivative PBC137 strains

| | | | |
|---|---|---|---|
| Strain ID | Genotype | Mutation | Mutation Description |
| 137 | WT | WT | Wild type Klebsiella variicola strain. |
| 137-1036 | ΔnifL::PinfC | ΔnifL::PinfC | Deletion of the nifL gene from 20bp after the ATG (start) to 87bp before the TGA (stop) of the gene. A 500bp fragment of the region upstream of the infC gene containing the promoter of the infC gene was inserted (PinfC) upstream of nifA replacing the deleted portion. |
| 137-3896 | ΔnifL::PinfC, ΔglnD_UTase deactivation, NC_nifA_copy ::Prm1.2 | ΔnifL::PinfC | Deletion of the nifL gene from 20bp after the ATG (start) to 87bp before the TGA (stop) of the gene. A 500bp fragment of the region upstream of the infC gene containing the promoter of the infC gene was inserted (PinfC) upstream of nifA replacing the deleted portion. |
| | | ΔglnD_UTase_ Deactivation | Deactivation of the uridylyltransferase (UT) domain of the bifunctional uridylyltransferase/uridylyl-removing enzyme, glnD, by mutating amino acid residues 90 and 91 from GG to DV as well as residue 104 from D to A. |
| | | NC-nifA_copy::Prm 1.2 | Insertion of a copy of the nifA gene into a noncoding region of 137. This copy is being driven by a 400bp promoter (Prm1.2) derived from a region upstream of the cspE gene. |
| 137-3890 | ΔnifL::Prm1.2, ΔglnE_{AR}-KO2, NtrC_D54A | ΔnifL::Prm1.2 | Deletion of the nifL gene from 20bp after the ATG (start) to 87bp before the TGA (stop) of the gene. A 400bp fragment from the region upstream of the cspE gene containing the promoter of the cspE gene was inserted (Prm1.2) upstream of nifA replacing the deleted portion. |
| | | ΔglnE_{AR}-KO2 | Deletion of 1647bp after the start codon of the glnE gene. |
| | | NtrC_D54A | Deactivation of the phosphorylation site of the DNA-binding transcriptional regulator NrtC by swapping the 54^{th} amino acid from aspartate to alanine (D to A). Disables the ability of NtrC to be phosphorylated. |

### Case I: Current Gen1 Microbe Providing 17 Lbs of N From Strain 137-1036

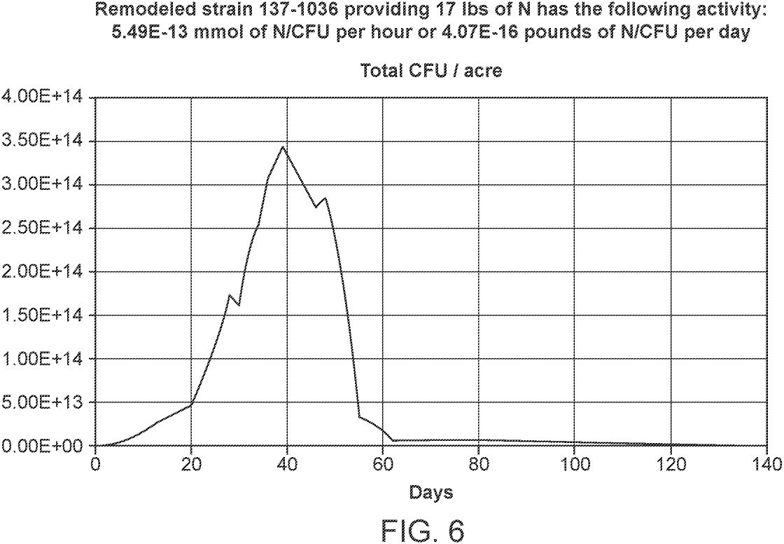

FIG. 6 depicts the colonization days 1-130 and the total CFU per acre of the non-intergeneric remodeled microbe of 137-1036, which was discussed previously. As mentioned, this microbe produces about 20 pounds of nitrogen per acre (in the aggregate) (17 pounds). The remodeled 137-1036 microbe has the following activity: 5.49E-13 mmol of N/CFU per hour or 4.07E-16 pounds of N/CFU per day.

### Case II: Current Gen1 Microbe Strain 137-1036 After Activity Improved 5-Fold to Provide First Half of N Requirement

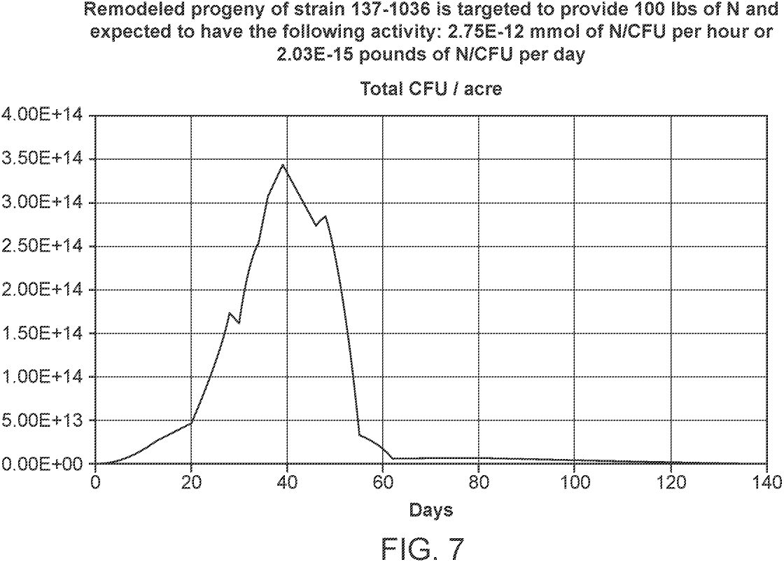

FIG. 7 depicts the colonization days 1-130 and the total CFU per acre of the proposed non-intergeneric remodeled microbe (progeny of 137-1036, see FIGS. 4A-4B and FIGS. 5A-5B for proposed genetic alteration features), which was discussed previously. As mentioned, this microbe is expected to produce about 100 pounds of nitrogen per acre (in the aggregate) (scenario “A”). The remodeled 137-1036 progeny microbe is targeted to have the following activity: 2.75E-12 mmol of N/CFU per hour or 2.03E-15 pounds of N/CFU per day. As noted above, since the filing of the provisional application, the features F2 and F3 have been incorporated and the activity of the remodeled strain 137-3890 with features F1—F3 is 4.03E-13 mmol of N/CFU per hour.

### Case III: Microbe With Later Stage Colonization With 5x Improved Activity

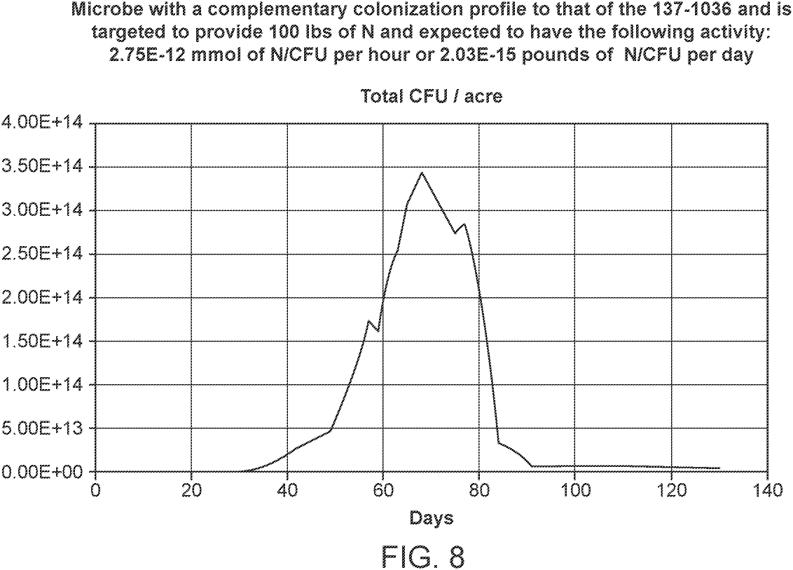

FIG. 8 depicts the colonization days 1-130 and the total CFU per acre of a proposed non-intergeneric remodeled microbe that has a complimentary colonization profile to the 137-1036 microbe. As mentioned, this microbe is expected to produce about 100 pounds of nitrogen per acre (in the aggregate) (scenario “B” in FIG. 3A), and should start colonizing at about the same time that the 137-1036 microbe begins to decline. The microbe is targeted to have the following activity: 2.75E-12 mmol of N/CFU per hour or 2.03E-15 pounds of N/CFU per day.

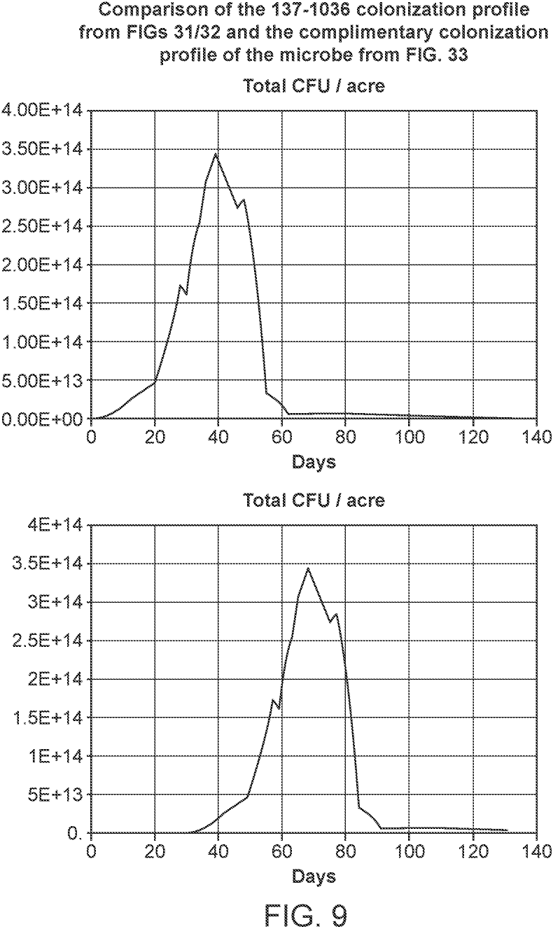

FIG. 9 provides the colonization profile of the 137-1036 in the top panel and the colonization profile of the microbe with a later stage/complimentary colonization dynamic in the bottom panel.

### Case IV: Combine Microbe From Case II and III Into a Consortia, or Find and Remodel a Single Microbe That Has the Depicted Colonization Profile and Stated Activity

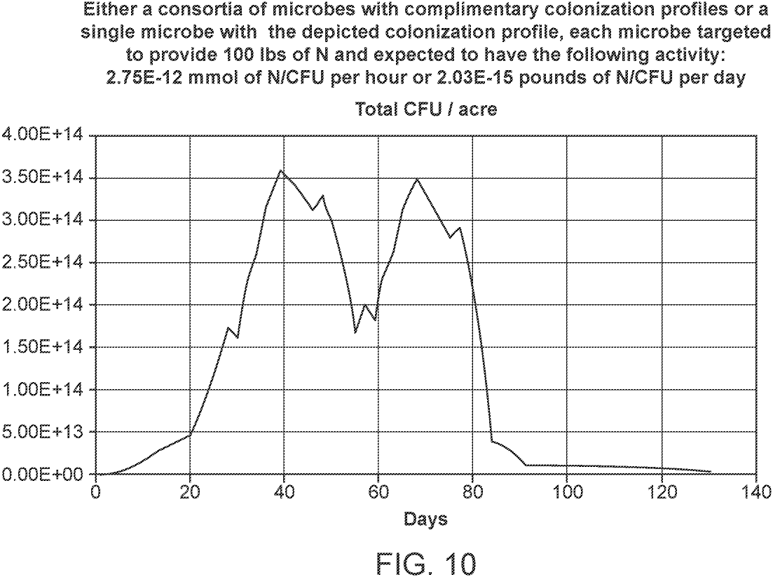

FIG. 10 depicts two scenarios: (1) the colonization days 1-130 and the total CFU per acre of a proposed consortia of non-intergeneric remodeled microbes that have a colonization profile as depicted in Case II and Case III explained above, or (2) the colonization days 1-130 and the total CFU per acre of a proposed single non-intergeneric remodeled microbe that has the depicted colonization profile. The microbe (whether two microbes in a consortia, or single microbe) is targeted to have the following activity: 2.75E-12 mmol of N/CFU per hour or 2.03E-15 pounds of N/CFU per day.

### Example 3: GMR Campaigns Utilizing Microbes With Distinct Spatial Colonization Patterns in the Corn Root Zone

As aforementioned in Example 2, the present disclosure provides a GMR campaign, which seeks to provide a farmer with a complete replacement for traditional synthetic fertilizer delivery. The “ecological sidedressing” discussed above in Example 1, which eliminates the need for a farmer to supply an in-season nitrogen application, is one step toward the ultimate goal of supplying a BNF product for cereal crops.

In order to remodel a microbe to be a successful BNF product for a cereal crop, it is paramount that the microbe colonizes a corn plant at a physiologically relevant time period of the corn’s growth cycle, as well as colonizing said corn plant to a sufficient degree.

The inventors have surprisingly discovered a functional genus of microbes, which have a desirable spatial colonization pattern, which make this group of microbes particularly useful for GMR campaigns.

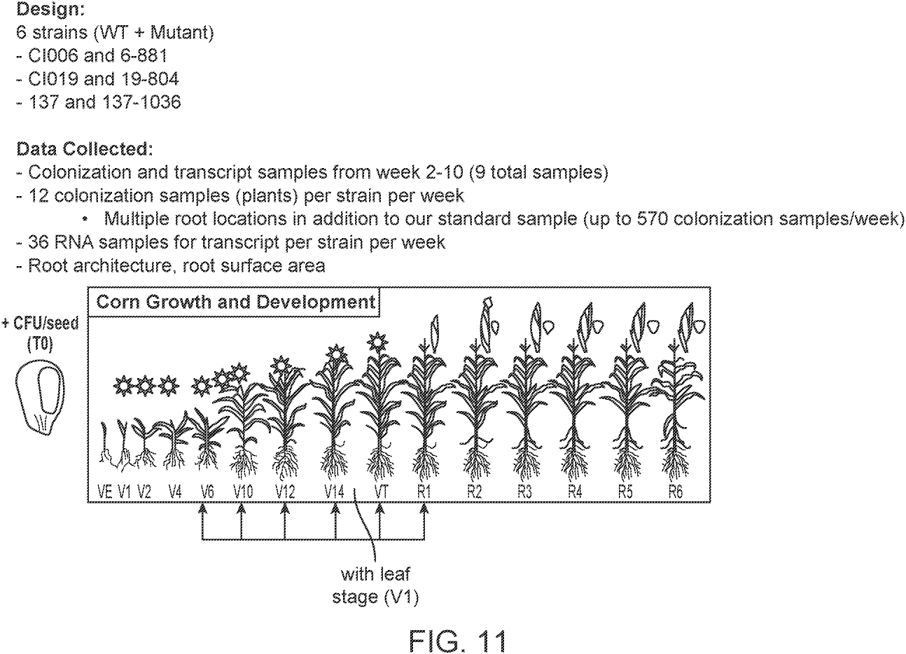

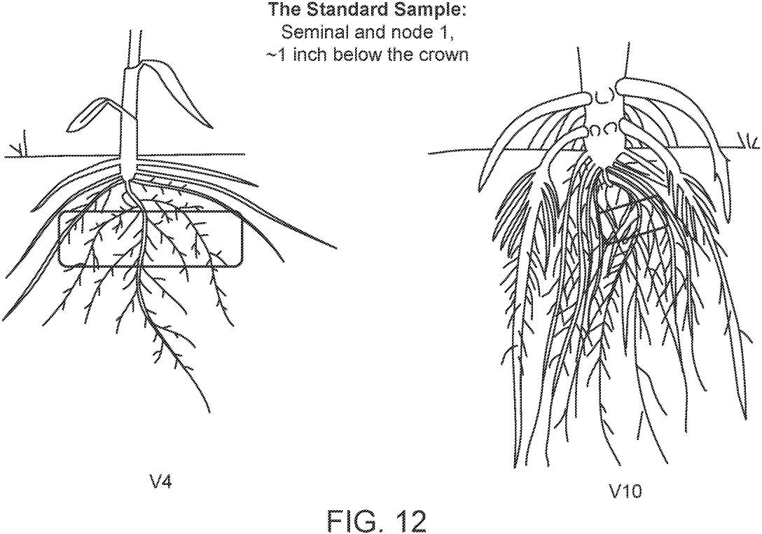

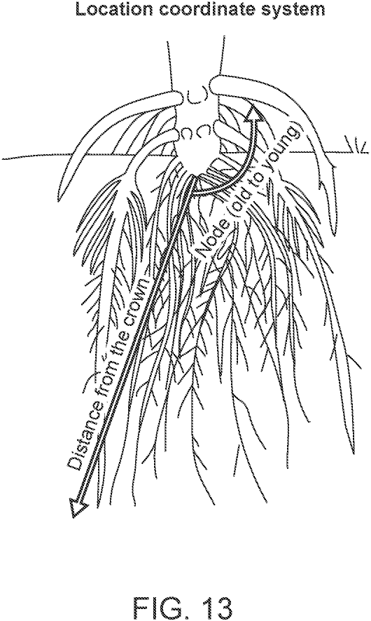

FIG. 11 sets forth the general experimental design utilized in this study, which entailed collecting colonization and transcript samples from corn over the course of 10 weeks. These samples allowed for the calculation of colonization ability of the microbes, as well as activity of the microbes. FIG. 12 and FIG. 13 provide a visual representation of aspects of the sampling scheme utilized in the experiment, which allows for differentiation of colonization patterns between a “standard” seminal node root sample and a more “peripheral” root sample.

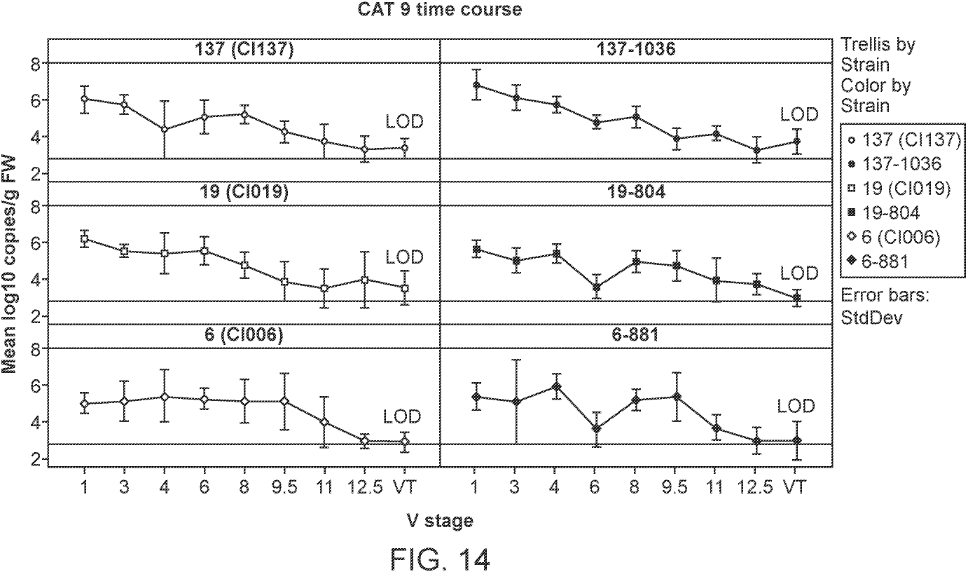

As can be seen in FIG. 14, the WT 137 (Klebsiella variicola), 019 (Rahnella aquatilis), and 006 (Kosakonia sacchari), all have a similar colonization pattern, which demonstrates a dropoff in colonization toward the later weeks. This pattern is mirrored in the remodeled forms of each strain, which are depicted in the right hand side of the graphic

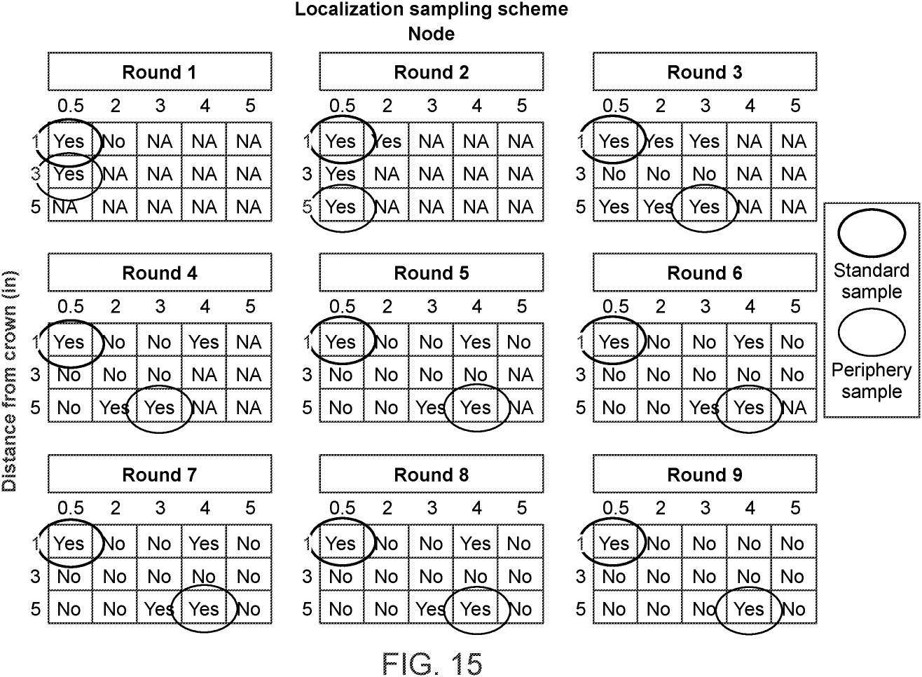

FIG. 15 depicts the experimental scheme utilized to sample the corn roots. The plots: each square is a time point, the Y axis is the distance, and the X axis is the node. The standard sample was always collected along with the leading edge of growth. The periphery and intermediate samples changed week to week, but an attempt at consistency was made.

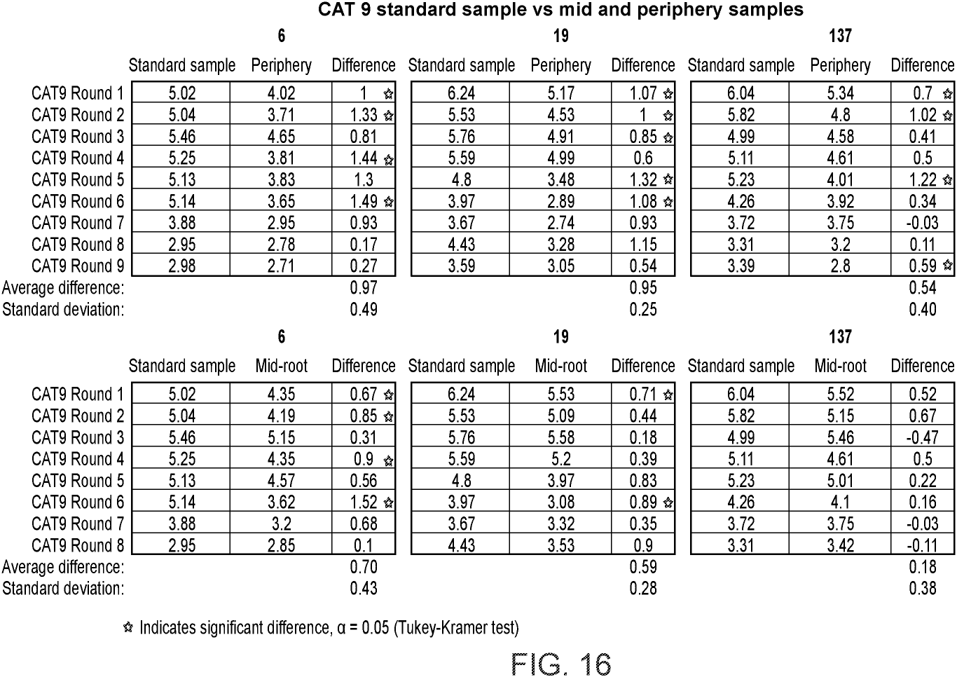

FIG. 16 depicts the overall results from the experiment, which utilized and averaged all the data taken in the sampling scheme of FIG. 15. As can be seen from FIG. 16, strain 137 maintains higher colonization in peripheral roots than strain 6 or strain 19. The ‘standard sample’ was most representative for this strain when compared to samples from other root locations.

### Example 4: Higher Corn Planting Density Enabled by Remodeled Microbes

Corn yields have increased significantly since the 1930s largely due to genetic improvement and better crop management. Grain yield is the product of the number of plants per acre, kernels per plant, and weight per kernel. Of the three components that make up grain yield, the number of plants per acre is the factor that the farmer has the most direct control over. Kernel number and kernel weight can be managed indirectly through proper fertility, weed, pest and disease management to optimize plant health, and weather also plays a major role. Currently the average U.S. corn planting density is just under 32,000 plants per acre and has increased 400 plants per acre per year since the 1960s.

However, ever-increasing planting populations are resulting in smaller and less expansive root systems available to acquire nutrients. Placing nutrients directly in the root zone at the right time using the correct source and rate increases the probability that roots will take up and utilize those nutrients.

Integrating this understanding of seeding rates, row spacing, and product placement with advanced fertility management practices such as applying the right source, right rate, right timing, and right place for nutrient management is critical to maximize grain yield and input efficiency at higher planting densities.

The microbes of the disclosure enable more densely planted corn crops, as the microbes live in intimate association with the plant (i.e. root surface) and provide the plant with a constant source of readily useable fixed atmospheric nitrogen.

The disclosure’s teachings of a BNF source for cereal crops will provide farmers with a tool that enables more densely planted acreage, as all the plants in the field will have a ready source of nitrogen delivered to their root systems throughout the growing season. This type of nitrogen delivery will not only remove the need for an in-season “sidedressing” application of nitrogen, but will also enable the farmer to realize a higher yield per acre due to the increased planting density per acre.

### Example 5: Reduced Infield Variability of Corn Crop Enabled by Remodeled Microbes

The present inventors have further determined that the microbes of the disclosure are able to improve yield stability and predictability through a more consistent and uniform delivery of nitrogen. The microbes of the disclosure enable reduced infield variability of a corn crop exposed to said microbes, which translates into improved yield stability and predictability for the farmer.

### Experimental Protocol for NDVI Field Trial

NDVI measurements were taken through satellite imaging about 1.5 months after corn planting to monitor the Normalized Difference Vegetation Index measurement. NDVI is calculated from the visible and near-infrared light reflected by vegetation. The remodeled microbe 137-1036 was applied to treat the corn, i.e. the remodeled Klebsiella variicola.

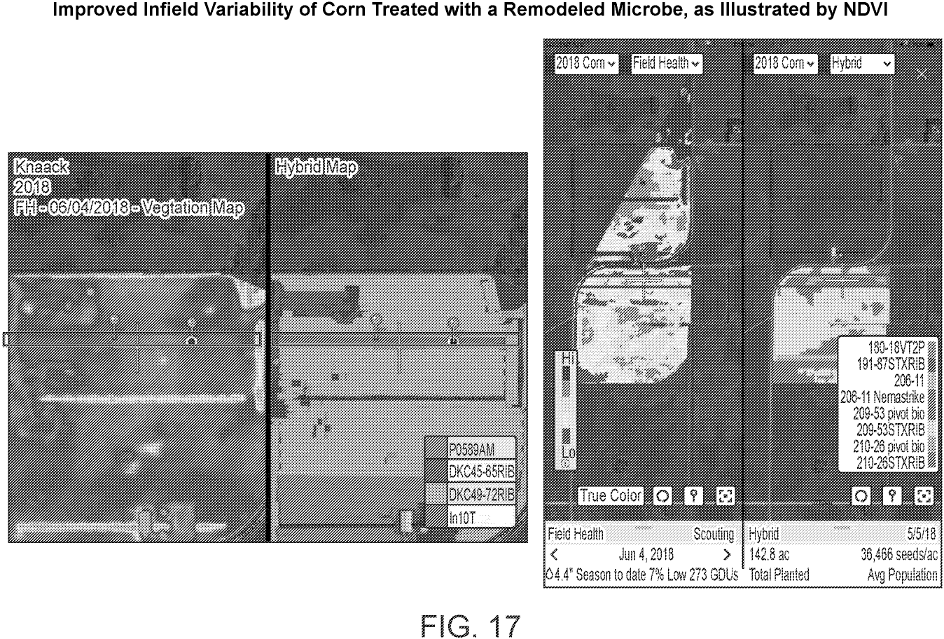

With respect to FIG. 17 that illustrates the results of the field experiment, healthy vegetation absorbs most of the visible light that hits it, and reflects a large portion of the near-infrared light. Unhealthy or sparse vegetation reflects more visible light and less near-infrared light.

In the two plots that are shown in FIG. 17, the microbes of the disclosure (137-1036) were applied to the field area plots demarcated with the “pins” (left panel) and the “cross markers” right panel. The treated area has also been illustrated with a square border. In both cases (left and right panels of FIG. 17), a more consistent NDVI measurement across the whole treated area was observed, compared to areas not treated with the 137-1036 microbe.

Data on mean yield of corn from a field trial showing reduced infield variability for the field treated with the remodeled strain of the present disclosure (137-1036 strain) compared to untreated field is shown in Table 7 below.

TABLE 7

| Average sidedress reduction lbs N/ac | Average PBM mean yield | Average check mean yield bpa | Average PBM sd yield | Average check sd yield bpa | [symbol] |
|---|---|---|---|---|---|
| 35 | 227.8 | 228.4 | 16.5 | 19.9 | [symbol] |

[symbol]

indicates text missing or illegible when filed

The data in Table 7 is an average from 5 different locations comparing untreated field (check) and ProveN (137-1036 strain) treated field (PBM). The untreated/check fields were not treated with the microbes of the present disclosure and had exogenous N applied. The PBM fields were treated with the microbes of the present disclosure, but did not have sidedress applied. As shown in Table 7, the PBM field needed 35 lbs less side-dressing (first column); at the same time, the mean yield from the PBM field and untreated field was similar. The standard deviation for the mean yield obtained from the PBM field is considerably less than that of the check (16.5 vs 19.9 bushels per acre (bpa)). The lesser standard deviation for the PBM-treated field indicates more uniform vegetation and reduced heterogeneity, compared to the control field which is consistent with the NDVI data shown in FIG. 17.

### Example 6: Nitrogen Delivery by Sustainable Nitrogen Producing Microbes Across Challenging Soil Types in Corn Fields

The present inventors determined that over the course of evaluating the performance of the presently disclosed nitrogen producing microbes across a variety of soil types and conditions, the microbes consistently colonized corn roots and supplied N to corn plants, even in challenging soil types where traditional N fertilizer was not very effective. The present study evaluated 47 different soil types in variable weather conditions across 13 states in the U.S., which revealed the microbes thrived in all of the evaluated soil types and weather conditions. In this study, the soil with a high sand content was considered a “challenging” or “problematic” soil type as growers can lose nitrogen in these type of coils quickly; whereas, the soil with a low sand content was considered a “typical” or “non-problematic” soil type. The % sand content of 47 evaluated soil types was measured; it was observed that 5 of them had a very high sand content. Specifically, 5 of the 47 evaluated soil types had an average sand content of about 50.90% and were considered a “challenging” or “problematic” soil type and the remaining soil types with an average of about 26.64% sand content were considered a “typical” or “non-problematic” soil type. The individual sand content of the 5 challenging soil types is listed in Table 8.

Growers typically lose nitrogen in heavy rains and/or challenging soil types. The microbes exhibited strong performance in a variety of challenging soil types, as well as soil exposed to heavy rains.

The data from field trials showing improvement in corn yield for challenging soil types treated with the remodeled microbes of the present disclosure compared to the same soil type not treated with the remodel microbes is summarized in Table 8 below. The column “Pivot Yield” in Table 8 shows the yield from the challenging soil type fields treated with the remodeled strains of the present disclosure. For challenging soil types, the remodeled microbes conferred a ~17 bushel per acre average advantage against fields in comparable conditions using only chemical nitrogen fertilizer. This superior improvement in yield in challenging soil types and soil exposed to heavy rains is surprising and unexpected, because under typical soil and weather conditions, the application of the microbes exhibited a ~7.7 bushel per acre advantage compared to fields without the microbes.

Utilizing the present microbes reduced the need for chemical fertilizer and delivers a return on investment to the growers who use the microbes, while decreasing the complexity and risk typically associated with chemical fertilizer use

As illustrated in Example 5 relating to reduced infield variability, as measured by NDVI, the current data of Example 6, demonstrating improved performance across a wide range of soil types, further illustrates that the microbes taught herein are able to lend yield predictability and reduce yield heterogeneity across a farmer’s field.

The ability for a farmer to realize relatively homogeneous yield gains across their growing acreage, even in acres normally susceptible to low yields, is a dramatic step forward in the art. Farmers will now be able to more reliably predict yields and realize value on acreage that traditionally would be low performing.

TABLE 8

| field.id | Soil Type Names | Texture Class | Organic Matter | Cation Exchange Coefficient | pH | % Sand | % Silt | % Clay | Saturated Hydrolic Coefficient | Erodability Factor | Drainage Class | Water Storage | Pivot Yield (bu/acre) | Untreated Yield (bu/acre) | Difference in Yield |
|---|---|---|---|---|---|---|---|---|---|---|---|---|---|---|---|
| 18PB12J1 | Kandota sandy loam, 2 to 6 percent slopes | Sandy loam | 0.486576 | 8.91133 | 6.582414 | 57.75961 | 17.0822 7 | 15.15813 | 10.00583 | 0.214759 | Well drained | 24.21 | 184.6102 | 171.8642 | 12.74599 |
| 18PB12K1 | Nicollet loam, 1 to 3 percent slopes | Loam | 1.332813 | 14.82493 | 7.404525 | 43.412 | 37.5132 5 | 19.07475 | 9.077525 | 0.342763 | Somew hat poorly drained | 28.04 | 219.4818 | 207.4651 | 12.01668 |
| 18PB1A1 | Hamerly-Tonka-Parnell complex, 0 to 3 percent slopes | Loam | 1.832989 | 21.28688 | 7.626457 | 31.62567 | 39.3931 3 | 28.9762 | 6.175194 | 0.301585 | Somew hat poorly drained | 24.64 | 257.7206 | 229.7333 | 27.98731 |
| 18PB12H1 | Fieldon-Canisteo loams | Loam | 2.730757 | 13.67336 | 7.11 | 53.23678 | 21.0803 3 | 15.68289 | 28.68618 | 0.202908 | Poorly drained | 21.26 | 214.953 | 195.3212 | 19.63179 |
| 18PB1E1 | Tracy sandy loam, 0 to 2 percent slopes | Sandy loam | 0.701942 | 6.156816 | 5.358657 | 68.46489 | 19.4701 2 | 12.06499 | 42.98403 | 0.1522 | Well drained | 21.03 | 195.4602 | 180.0739 | 15.38628 |

### Example 7: Improving Activity of Microbial Strains

In this example, several non-transgenic derivative strains of Klebsiella veiriicolct Wild type (WT) strain, CI137, were generated. First, the WT strain, CI137, was isolated from a rhizosphere, characterized, and domesticated.

Then, the nitrogen fixation trait of CI137 was rationally improved without the use of transgenes. To test whether the nitrogen fixation trait of the WT strain can be improved, various genes involved in nitrogen fixation as described throughout this application were targeted to engineer non-intergeneric mutations, the engineered/remodeled microbes were analyzed for nitrogen fixation, and the engineering and the analytics steps were iterated to test whether further improvements can be made in the nitrogen fixation ability. Using this iterative approach, beneficial mutations were stacked to increase the nitrogen fixation ability.

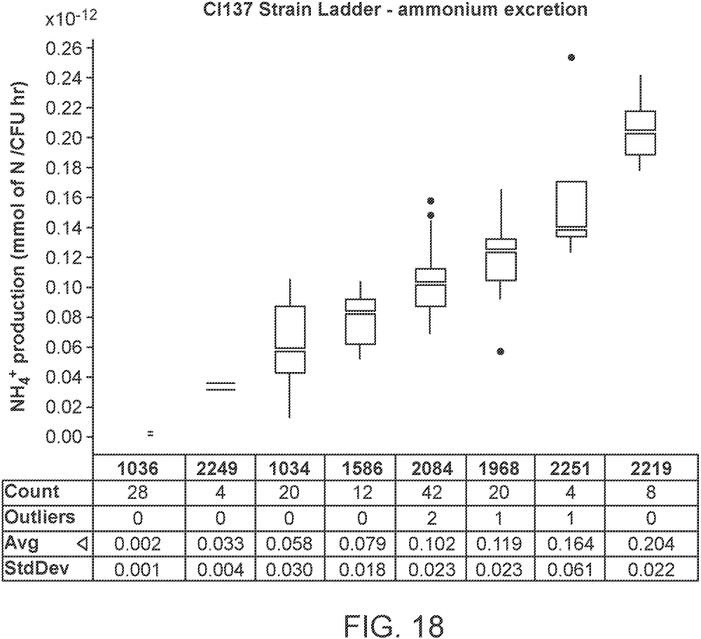

Non-intergeneric mutations made through this iterative remodeling process to generate remodeled CI137 strains that showed improvement in nitrogen fixation are summarized in Table 9 below. The stepwise improvement in the nitrogen fixation trait of the remodeled strains is shown in FIG. 18.

TABLE 9

137 Strain and Mutation Description

| | | | | | | |
|---|---|---|---|---|---|---|
| Strain | Strain ID | SEQ ID NO | Genotype | Mutation | Mutation Description | Associated Novel Junction If Applicable |
| | 137-1036 | | ΔnifL::PinfC | ΔnifL::PinfC | Deletion of the nifL gene from 20bp after the ATG (start) to 87bp before the TGA (stop) of the gene. A 500bp fragment of the region upstream of the infC gene was inserted (PinfC) upstream of nifA replacing the deleted portion. | |
| | 137-1034 | | ΔglnE_{AR}-KO2 | ΔglnE_{AR}-KO2 | Deletion of 1647bp after the start codon of the glnE gene. | |
| | 137-2249 | | ΔnifL::PinfC | ΔnifL::PinfC | Deletion of the nifL gene from 20bp after the ATG (start) to 87bp before the TGA (stop) of the gene. A 500bp fragment of the region upstream of the infC gene was inserted (PinfC) upstream of nifA replacing the deleted portion. | |
| | | | glnE_{AR}-DxD | glnE_{AR}-DxD | Modification of the “GAT” found 513bp after the start codon of glnE to a “GCG” codon. | |
| | 137-1968 | | ΔnifL::Prm8. 2 | ΔnifL::Prm8.2 | Deletion of the nifL gene from 20bp after the ATG (start) to 87bp before the TGA (stop) of the gene. A 299bp fragment (Prm8.2), found 77bp after the start codon of nlpI to 376bp after the start codon of nlpI was inserted upstream of nifA replacing the deleted portion. | |
| | | | ΔglnE_{AR}- KO2 | ΔglnE_{AR}-KO2 | Deletion of 1647bp after the start codon of the glnE gene. | |
| | 137-1586 | | ΔnifL::PinfC | ΔnifL::PinfC | Deletion of the nifL gene from 20bp after the ATG (start) to 87bp before the TGA (stop) of the gene. A 500bp fragment of the region upstream of the infC gene was inserted (PinfC) upstream of nifA replacing the deleted portion. | |
| | | | ΔglnE_{AR}- KO2 | ΔglnE_{AR}-KO2 | Deletion of 1647bp after the start codon of the glnE gene. | |
| | 137-2084 | | ΔnifL::Prm1. 2 | ΔnifL::Prm1.2 | Deletion of the nifL gene from 20bp after the ATG (start) to 87bp before the TGA (stop) of the gene. A 400bp fragment from the region upstream of the cspE gene was inserted (Prm1.2) upstream of nifA replacing the deleted portion. | |
| | | | ΔglnE_{AR}- KO2 | ΔglnE_{AR}-KO2 | Deletion of 1647bp after the start codon of the glnE gene. | |
| | 137-2251 | | ΔnifL::Prm1. 2 | ΔnifL::Prm1.2 | Deletion of the nifL gene from 20bp after the ATG (start) to 87bp before the TGA (stop) of the gene. A 400bp fragment from the region upstream of the cspE gene was inserted (Prm1.2) upstream of nifA replacing the deleted portion. | |
| | | | ΔglnE_{AR}-KO2 | ΔglnE_{AR}-KO2 | Deletion of 1647bp after the start codon of the glnE gene. | |
| | | | rpoN-Prm8.2 | rpoN-Prm8.2 | Deletion of the 47bo between ibtB2 and rpoN and insertion of a fragment (Prm8.2), found 77bp after the start codon of nlpI to 376bp after the start codon of nlpI, directly upstream of rpoN. | |
| | 137-2219 | | ΔnifL::Prm1. 2 | ΔnifL::Prm1.2 | Deletion of the nifL gene from 20bp after the ATG (start) to 87bp before the TGA (stop) of the gene. A 400bp fragment from the region upstream of the cspE gene was inserted (Prm1.2) upstream of nifA replacing the deleted portion. | |
| | | | ΔglnE_{AR}- KO2 | ΔglnE_{AR}-KO2 | Deletion of 1647bp after the start codon of the glnE gene. | |
| | | | ΔglnD_{ACT½} | ΔglnD_{ACT½} | Deletion of the 546bp before the stop codon of the glnD gene. | |

The feature sets indicated in Table 10 correspond to the Features List in FIGS. 4A-4B, which recite F0, F1, F2, F3, F4, F5, and F6. The features amount to targeted improvements in strains to facilitate reduced exogenous nitrogen use in fields or complete replacement of exogenous nitrogen use in fields. The improvement in nitrogen fixation exhibited by the strains listed in Table 10 is shown in FIG. 18.

TABLE 10

Ammonium Excretion in Modified Cells

| Strain ID | Genotype | Feature Sets |
|---|---|---|
| 137-1036 | ΔniflL::PinfC | F1 |
| 137-2249 | ΔnifL::PinfC, glnE_{AR}-DxD | F1, F2 |
| 137-1034 | ΔglnE_{AR}-KO2 | F2 |
| 137-1586 | ΔnifL::PinfC, ΔglnE_{AR}-KO2 | F1, F2 |
| 137-2084 | ΔnifL::Prm1.2, ΔglnE_{AR}-KO2 | F1, F2 |
| 137-1968 | ΔnifL::Prm8.2, ΔglnE_{AR}-KO2 | F1, F2 |
| 137-2251 | ΔnifL::Prm1.2, rpoN-Prm8.2 | F1, F4 |
| 137-2219 | ΔnifL::Prm1.2, ΔglnE_{AR}-KO2, ΔglnD_{ACT½} | F1, F2, F3 |

### Example 8: Improved Consistency of Crop Yield Through Biological Nitrogen Fixation

As with Example 5 “Reduced Infield Variability of Corn Crop Enabled by Remodeled Microbes,” the present example provides extensive data, across a range of study sites and field conditions, demonstrating improved consistency of corn yield and reduced infield variability across a farmer’s acreage.

Nitrogen is an important nutrient for cereal crops. Nitrogen is usually provided in the form of fertilizer that is applied across a field in which crops are planted. Because applied fertilizer can be lost to the environment (e.g., due to weather effects), this can lead to inconsistencies in crop yield. The current example demonstrates the ability of remodeled microbes of the disclosure to increase consistency of crop yield by providing nitrogen through biological nitrogen fixation to a host plant across a wide variety of environmental conditions. The microbes of the disclosure allow the farmer to reliably predict yield for a crop, even in the face of challenging soil and weather conditions. Thus, improved consistency of crop yield is expected to provide significant benefits to farmers who can now more reliably obtain yield from their fields regardless of external field (e.g., weather or soil) conditions.

### Field Trial Procedures

Performance of a remodeled microbe of the disclosure, i.e. 137-1036, was evaluated in multiple farmer fields in 2019. Corn yields with the grower standard nitrogen fertilization practice were compared to corn yields with 137-1036 added to the system as an in-furrow application at planting. Farmers were instructed to split fields in half with a 137-1036 treated area on one side of the field and the Grower Standard Practice (GSP) on the other. Trial participants provided digital as-planted (planter monitor) and harvest (combine yield monitor) maps identifying the two treatment zones. ArcGIS software was used to analyze data and compare yield differences between the zones.

### Data Analysis

Uniform crop development is an important factor in maximizing yields and an important driver of within field yield variance. Corn is more responsive to nitrogen than other nutrients. Consequently, differences in nitrogen availability within fields contributes greatly to yield variance. The product containing the remodeled microbe 137-1036 can serve as a baseline nitrogen source that doesn’t leach and delivers nitrogen to the corn plant in a more consistent and reliable manor compared to traditional synthetic nitrogen sources.

Yield data from harvest combine monitors on 34 farms collected during the 2019 harvest season were used to examine changes in yield variability between field areas treated with 137-1036 and untreated control areas by analyzing the yield homogeneity of variance and standard deviation.

Combine data was put through an initial QC check having been standardized to a common format. Of the 34 farms, data from 3 sites were discarded due to serious defects in field conditions, on-farm management, or data collection issues that rendered the comparison between 137-1036 and untreated control as unrepresentative.

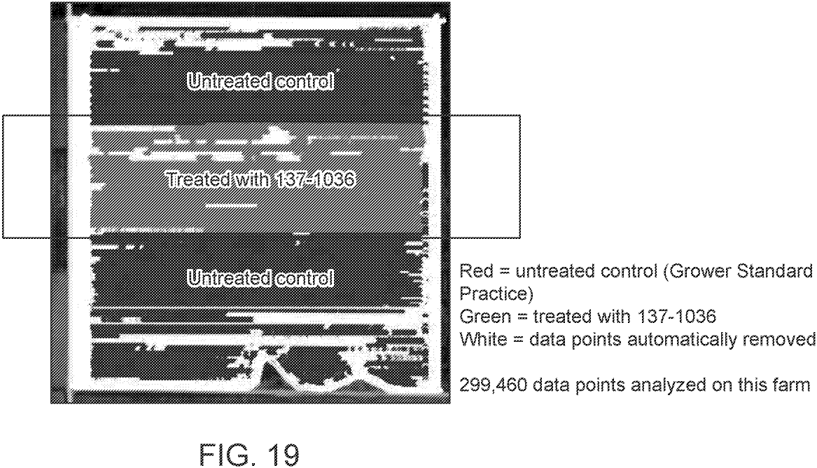

Additional QA/QC procedures were applied to combine data ensuring representative comparisons from both 137-1036 and untreated field regions. Header rows, which are typically lower in yield, more prone to damage and have a varying incident solar radiation profile, were removed from field data sets. This can be seen in FIG. 19 where the perimeter of the field is white. Furthermore, the most reliable data from combine harvest monitors occurs in areas where the combine is moving at a steady velocity. Thus, data points were removed with automated filters where the combine was accelerating or where the combine had to slow down to pass obstacles like field drains or terraces.

FIG. 19 depicts the data points analyzed from an example field. The remaining 3.7 million data points from 31 farms were used for the analysis presented here. Table 11 summarizes the characteristics of this large dataset.

TABLE 11

Summary of data analyzed by treatment area

| | untreated control | 137-1036 treated | Total |
|---|---|---|---|
| Total yield data points | 1,873,663 | 1,842,127 | 3,715,790 |
| Total area (acres) | 2,152 | 1,631 | 3,783 |
| Total harvest volume (bushels) | 428,573 | 341,949 | 770,522 |
| Average data points/farm | 60,441 | 59,423 | 119,864 |
| Average bushels/acre | 199.1 | 209.7 | 203.7 |
| Average acres/farm | 69.4 | 52.6 | 122.0 |

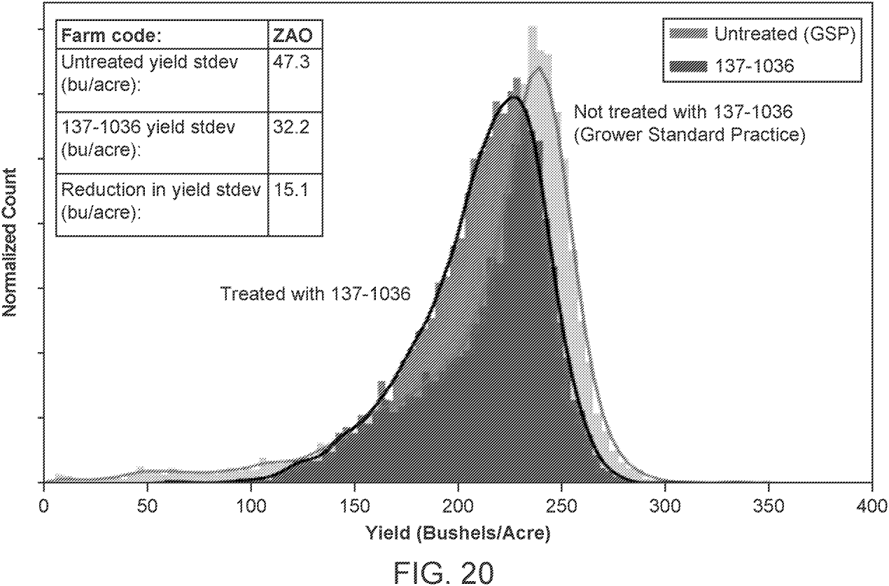

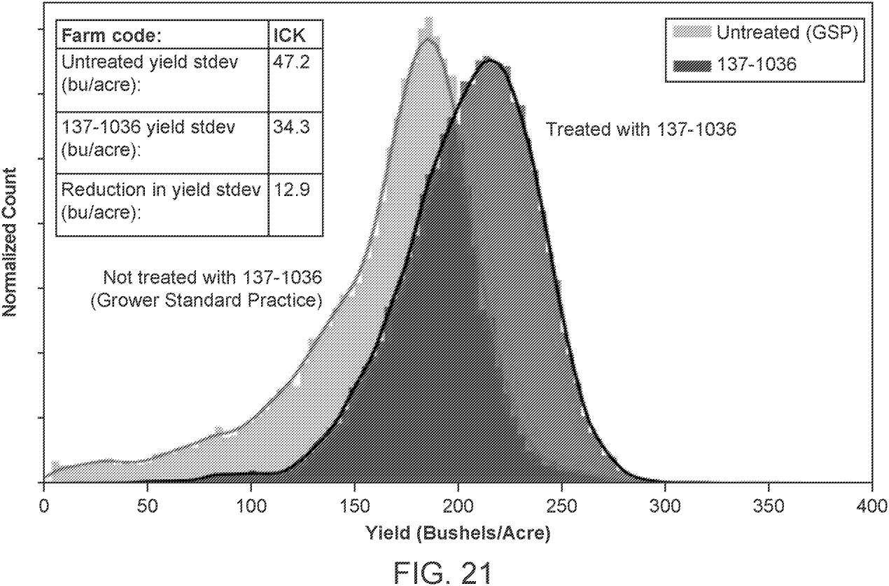

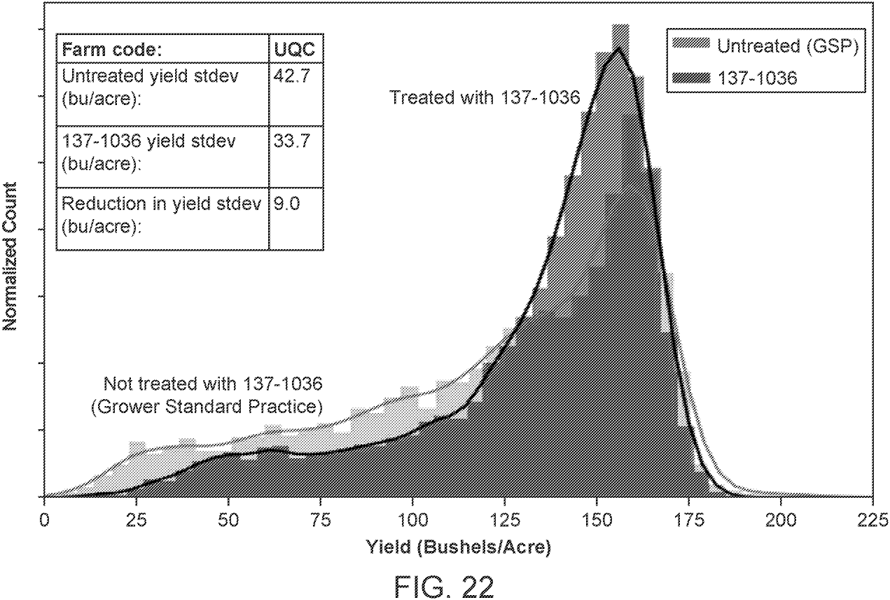

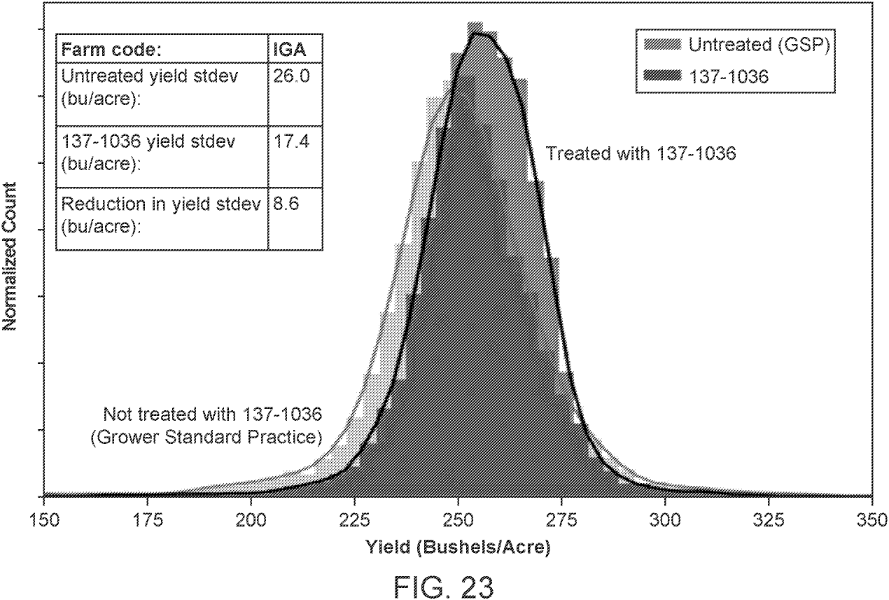

On each individual farm, the difference in standard deviations for yield (bushels/acre) between 137-1036 treated and the untreated control was calculated. FIGS. 20-24 show examples of the distribution of yield data for a single farm. For example in FIG. 20, the width of the 137-1036 treated distribution is visibly smaller than the untreated distribution, and it has a standard deviation 15.1 bushels/acre less than the untreated control.

We found a reduction of yield variability (an improvement in consistency) in 64% of farms. The median reduction was 1.65 bushels/acre, and the mean reduction was 2.22 bushels/acre.

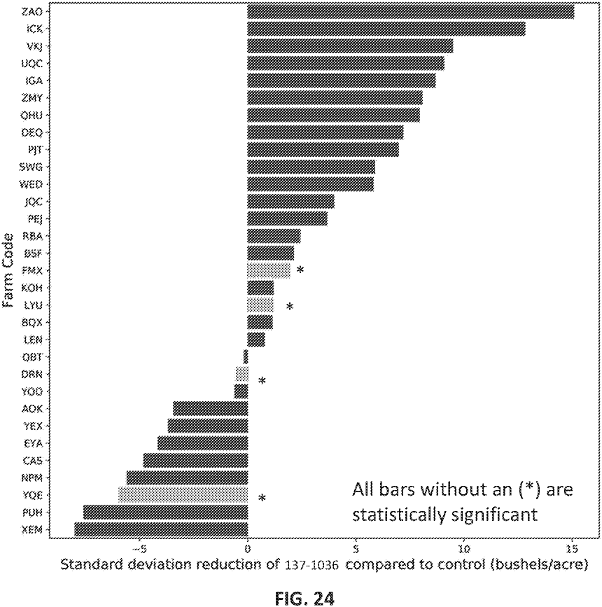

Across the 31 farms, 20 showed a reduction in standard deviation of yield for 1367-1036 treated compared to the untreated control, a 64% win rate. FIG. 24 summarizes these differences by farm showing the control standard deviation in yield minus the 137-1036 treated standard deviations in yield. On 87% of farms the difference in yield variance from field regions treated with 137-1036 compared to untreated regions was significant. Only four of the bars in FIG. 24 are colored grey, indicating differences between 137-1036 treated and untreated yield variance that were not significant (denoted by an *). Significance was determined by application of Levene’s test for equal variances at the 95% significance level.

TABLE 12

Further Descriptions of Strains from the Disclosure

| Strain ID | Lineage | Mutagenic DNA Description | Genotype | Accession Number |
|---|---|---|---|---|
| CI006 | CI006 | Wildtype parent Kosakonia saccari | WT | 201701001 |
| CI137 | CI137 | Wildtype parent Klebsiella variicola | WT | 201708001 |
| CI910 | CI910 | Wildtype parent Metakosakonia intestini | WT | PTA-126585 |
| CI8 | CIB | Wildtype parent Paraburkholderia tropica | WT | PTA-126582 |
| CI41 | CI41 | Wildtype parent Paenibacillus polymyxa | WT | PTA-126581 |
| CI3069 | CI3069 | Wildtype parent Herbaspirillum aquaticum | WT | PTA-126583 |
| 6-403 | Mutant of CI006 | Disruption of nifL gene with a fragment of the region upstream of the 1pp gene inserted (Prm1) upstream of nifA. Deletion of the 1647bp after the start codon of the glnE gene containing the adenylyl-removing domain of glutamate-ammonia-ligase adenylyltransferase (ΔglnE-AR KO2). | ΔnifL::Prm1, ΔglnE-AR_KO2 | 201708004 |
| 6-2425 | Mutant of CI006 | Disruption of nifL gene with a fragment of the region upstream of the 1pp gene inserted (Prm1) upstream of nifA. Deletion of the 987bp after the start codon of the glnD gene containing the uridylyltransferase (UT) domain of the bifunctional uridylyltransferase/uridylyl-removing enzyme (ΔglnD-UT truncation) | ΔnifT::Prm1, ΔglnD_UT_truncation | PTA-126575 |
| 6-2634 | Mutant of CI006 | Disruption of nifL gene with a fragment of the region upstream of the 1pp gene inserted (Prm1) upstream of nifA. Deactivation of the uridylyltransferase (UT) domain of the bifunctional uridylyltransferase/uridylyl-removing enzyme, glnD, by | ΔnifL::Prm1, ΔglnD_UT_deactivation | PTA-126576 |
| | | mutating amino acid residues 90 and 91 from GG to DV as well as residue 104 from D to A. | | |
| 137-1034 | Mutant of CI137 | Deletion of the 1647bp after the start codon of the glnE gene containing the adenylyl-removing domain of glutamate-ammonia-ligase adenylyltransferase (ΔglnE-AR KO2). | ΔglnE-AR_KO2 | 201712001 |
| 137-2084 | Mutant of CI137 | Disruption of nifL gene with a fragment of the region upstream of the cspE gene inserted (Prm1.2) upstream of nifA. Deletion of the 1647bp after the start codon of the glnE gene containing the adenylyl-removing domain of glutamate-ammonia-ligase adenylyltransferase (ΔglnE-AR KO2). | ΔnifL::Prm1.2 ΔglnE-AR_KO2 | |
| 137-1968 | | Deletion of the native dctA1 promoter and insertion of a fragment (Prm8.2), found 77bp after the start codon of nlpI to 376bp after the start codon of nlpI, directly upstream of dctA1. Deletion of the 1647bp after the start codon of the glnE gene containing the adenylyl-removing domain of glutamate-ammonia-ligase adenylyltransferase (ΔglnE-AR_KO2). | ΔnifL::P8.2, ΔglnE-AR_KO2 | PTA-126577 |
| 137-2219 | Mutant of CI137 | Disruption of nifL gene with a fragment of the region upstream of the cspE gene inserted (Prm1.2) upstream of nifA. Deletion of the 1647bp after the start codon of the glnE gene containing the adenylyl-removing domain of glutamate-ammonia-ligase adenylyltransferase (ΔglnE-AR KO2). Deletion of the | ΔnifL::Prm1.2 ΔglnE-AR_KO2, ΔglnD_ACT12_truncation | PTA-126578 |
| | | 546bp before the stop codon of the glnD gene containing the ACT½ domain of the bifunctional uridylyltransferase/uridylyl-removing enzyme (ΔglnD-ACT12_truncation) | | |
| 137-2237 | Mutant of CI137 | Disruption of nifL gene with a fragment of the region upstream of the cspE gene inserted (Prm1.2) upstream of nifA. Deletion of the 1647bp after the start codon of the glnE gene containing the adenylyl-removing domain of glutamate-ammonia-ligase adenylyltransferase (ΔglnE-AR_KO2). Deletion of the native glsA2 promoter and insertion of a fragment (Prm1.2) directly upstream of the glsA2 CDS. | ΔnifL::Prm1.2, ΔglnE-AR_KO2, glsA2::Prm1.2 | PTA-126579 |
| 137-2285 | Mutant of CI137 | Disruption of nifL gene with a fragment of the region upstream of the cspE gene inserted (Prm1.2) upstream of nifA. Deletion of the 1647bp after the start codon of the glnE gene containing the adenylyl-removing domain of glutamate-ammonia-ligase adenylyltransferase (ΔglnE-AR_KO2). Deletion of the native rpoN promoter and insertion of a fragment (Prm1.2) directly upstream of the rpoN CDS. | ΔnifL::Prm1.2, ΔglnE-AR_KO2, rpoN::Prm1.2 | PTA-126580 |
| 910-3994 | Mutant of CI910 | Disruption of nifL gene with a fragment of the region upstream of the rmF gene inserted (Prm2.1) upstream of nifA. Deletion of the 1647bp after the start codon of the glnE gene containing the adenylyl-removing domain of glutamate-ammonia-ligase | ΔnifL::Prm2.1, ΔglnE-AR_KO2, glsA2::Prm1.1 | PTA-126588 |
| | | adenylyltransferase (ΔglnE-AR_KO2). Deletion of the native glsA2 promoter and insertion of a fragment upstream of the csrA gene (Prm1.1) directly upstream of the glsA2 CDS. | | |
| 910-3963 | Mutant of CI910 | Disruption of nifL gene with a fragment of the region upstream of the rmF gene inserted (Prm2.1) upstream of nifA. Deletion of the 1647bp after the start codon of the glnE gene containing the adenylyl-removing domain of glutamate-ammonia-ligase adenylyltransferase (ΔglnE-AR_KO2). Deletion of the 987bp after the start codon of the glnD gene containing the uridylyltransferase (UT) domain of the bifunctional uridylyltransferase/uridylyl-removing enzyme (AglnD-UT truncation) | ΔnifL::Prm2.1, ΔglnE-AR_KO2, ΔglnD_UT_truncation | PTA-126586 |
| 910-3655 | Mutant of CI910 | Disruption of nifL gene with a fragment of the region upstream of the rmF gene inserted (Prm2.1) upstream of nifA. Deletion of the 1647bp after the start codon of the glnE gene containing the adenylyl-removing domain of glutamate-ammonia-ligase adenylyltransferase (ΔglnE-AR_KO2). | ΔnifL::Prm2.1, ΔglnE-AR_KO2 | PTA-126584 |
| 910-3961 | Mutant of CI910 | Disruption of nifL gene with a fragment of the region upstream of the rmF gene inserted (Prm2.1) upstream of nifA. Deletion of the 987bp after the start codon of the glnD gene containing the uridylyltransferase (UT) domain of the bifunctional uridylyltransferase/uridylyl-removing enzyme (ΔglnD-UT truncation). | ΔnifL::Prm2.1, ΔglnD_UT_truncation | PTA-126587 |

### Example 9 - Improved Plant Nitrogen Consistency

Two experiments were performed in Illinois in 2019 that show preliminary evidence that KV137 reduces variance in plant nitrogen. While the results were not statistically significant, the reductions were substantial. Significantly expanded measurement being conducted in 2020 will allow a statistically significant conclusion to be drawn.

In Experiment 1, there were three locations were used with two nitrogen rates: 0 lbs applied and full fertilizer based on local soil test. Whole plant nitrogen was measured at V6 and at maturity (R6). Yield and grain nitrogen content were measured at maturity. Individual plots were quality checked based on agronomic issues and discarded or included in the analysis accordingly. In-furrow applications of Proven resulted in positive yield responses at five of the six yield environments. For more information, please see the Experiment 1 Details section below.

Each location and nitrogen rate was analyzed separately for the difference in variance between the treatment and control. For the four traits measured, the reduction in variance ranged between 17% and 21% even though the average change in value was less than 3% for all traits. See Table 13.

TABLE 13

| | |
|---|---|
| Trait at Growth Stage | Change in Variance |
| Plant N mass at V6: | 18% decrease |
| Grain mass at R6: | 17% decrease |
| Grain N masss R6 | 17% decrease |
| Total N mass at R6 | 21% decrease |

These variances are very consistent. This suggests that the early reduction in nitrogen variance at V6 may result in the low variance in yield and end of season nitrogen at R6.

In the Experiment 2, one location was used at 5 fertilizer rates (0, 40, 80, 120, 200 lbs/acre). 8 replicates were used with 1 untreated plot and 1 plot with ProveN per replicate. Whole plant nitrogen was measured at the V8 and R1 (flowering) growth stages. Yield and grain protein % were measured at maturity. Using a mixed-effect model with replicate as a random effect and treatment as a fixed effect, a significant increase in plant nitrogen was measured at V8 due to KV137 (ProveN). See Table 14. No significant effect was observed in whole plant nitrogen at R1, yield at R6, or grain protein content at R6 due to ProveN.

TABLE 14

Test of fixed effects for partitioned and total plant measures of dry biomass, N concentration, and N content at the V8 growth stage as influenced by Proven application and N fertilizer rate for corn grown at Champaign, IL in 2019.

| Source of Variation | Leaf Biomass | Stalk Biomass | Total Biomass | Leaf N Content | Stalk N Content | Total N Content | Leaf N Conc | Stalk N Conc |
|---|---|---|---|---|---|---|---|---|
| P > F | | | | | | | | |
| N Rate (N) | 0.0002 | 0.0020 | 0.0003 | <0.0001 | <0.0001 | <0.0001 | <0.0001 | <0.0001 |
| Proven (P) | 0.0798 | 0.1344 | 0.0889 | 0.0735 | 0.0601 | 0.0364 | 0.9837 | 0.7148 |
| N x P | 0.7554 | 0.8546 | 0.8020 | 0.3814 | 0.7599 | 0.5130 | 0.5819 | 0.8414 |

At the V8 growth stage nitrogen variance was reduced 21%. This variance change was not observed later in the growing season at R1, though a modest but not significant improvement in yield was observed at harvest. See Table 15. Early performance on the metric of plant nitrogen improvement and variance, while evidence of biological nitrogen fixation, do not always lead to end-of-season yield improvements.

TABLE 15

| | |
|---|---|
| Trait at Growth Stage | Change in Variance |
| Plant N mass at V8 | 21% decrease |
| Plant N mass at R1 | 31% increase |
| Grain N mass at R6 | 10% decrease |
| Grain mass at R6 | 2% decrease |

### Experiment 1 Details

### Experimental Design

The fields were arranged as split-block designs, with fertility plans as main plots, and biological applications as sub-plots. Each location contained six replications. Plots consisted of four 30-inch wide, 37.5 feet long rows with a 2.5 foot walk alley between each range of plots. The fertilized blocks received N, P, and K pre-plant broadcast-applied using a Gandy Drop Spreader (Gandy, Owatonna, Minnesota) and incorporated with a standard harrow. Nitrogen was provided as dry urea at a rate of 160 lbs N acre-1. The phosphorus source was MicroEssentialsSZ (MESZ, 12-40-0-10S-1Z), applied at 75 lbs P2O5 acre-1 (additional 22.5 lbs N acre-1). Aspire (0-0-58-0.5B), the potassium source, was applied at 60 lbs K2O acre-1. A corn hybrid responsive to management (DK64-8-1) was planted at 34,000 plants per acre at all sites, 2nd June 2019 (Champaign), 6th June 2019 (Ewing), and 8th June 2019 (Yorkville).

### Treatment Applications

In-furrow treatments were blended with water for a total application volume of 8 gallons acre-1 and implemented with a planter-attached liquid starter applicator system (Surefire Ag Systems, Atwood, Kansas). Fertilizer and in-furrow treatments were applied 2nd June 2019 (Champaign), 6th June 2019 (Ewing), and 8th June 2019 (Yorkville).

### Measured Parameters

Soil samples (0 - 6″ depth) were taken from the plot areas prior to planting to assess fertility levels at each site (Table 14). Above ground plant samples were collected at both the V6 (vegetative 6-leaf) and R6 (physiological maturity) growth stages. The V6 sampling was done by excising six plants at the soil surface from plot rows one and four (three plants from each row) on 2nd July 2019 (Champaign), 4th July 2019 (Ewing), and 9th July 2019 (Yorkville). Samples were then dried to 0% moisture in a forced air oven at 75° C. and weighed for shoot biomass acre-1. Total above-ground plant biomass acre-1 was calculated based on the target planting stand of 34,000 plants acre-1. Once weighed, samples were ground to pass through a 2 mm screen using a Wiley Mill (Thomas Scientific, Swedesboro, New Jersey) and analyzed for nutrient concentrations (N, P, K, Ca, Mg, S, Zn, Mn, Cu, and B). Nutrient analysis was conducted by A & L Great Lakes Laboratories (Fort Wayne, Indiana). Total above-ground nutrient uptake at the V6 growth stage was calculated based on nutrient concentrations and total plant biomass acre-1.

### Experiment 2 Details

### Research Approach

The experiment was implemented during the 2019 growing season at the Crop Sciences Research and Education Center of the University of Illinois at Tirbana-Champaign. This location has been maintained weed- and disease-free, is a level and well-drained Drummer silty clay loam, and is well-suited to provide evenly distributed soil fertility, pH, soil organic matter, and water availability. Experimental units were plots eight rows wide and 37.5 feet in length with 30-inch row spacing. Rows 2, 3, 6, and 7 were used for sampling, while the middle rows (4 and 5) were used for yield. Plots were planted on 2 Jun. 2019 in Champaign, IL. Soybean was the previous crop and conventional tillage was used. A corn hybrid previously shown to be responsive to nitrogen (N), Golden Harvest G12W66, was grown at a population of approximately 36,000 plants/acre to assess the role of a N-fixing bacteria (Proven) applied in-furrow at planting. Plots were arranged using a random complete block design with eight replications, adding up to a total of 80 plots.

### Treatment Applications

The treatments were designed to determine the effectiveness of Proven applications combined with differing rates of nitrogen from zero, or limiting, to typically sufficient. Proven was provided in-furrow at planting to half of the plots at a rate of 2 L/acre, with the other half of the plots left untreated. Urea applied at pre-plant was broadcasted and incorporated into the soil at rates of either 0, 40, 80, 120, or 200 lbs N/acre.

### Parameters Measured

Soil samples (0″-12″ deep) were obtained from plot areas prior to planting and analyzed (A & L Great Lakes Laboratories, Fort Wayne, IN) to determine fertility levels (Table 13).

The center two rows of each plot were mechanically harvested for determination of grain yield and harvest moisture, and the yield subsequently standardized to bushels/acre at 15.5% moisture. The harvest date for this trial was 23 Oct. 2019. Subsamples of the harvested grain were evaluated for yield components (individual kernel number and kernel weight) and for grain quality (protein, oil, and starch concentrations) by NIT (Infratec 1241, Foss North America, Eden Prairie, MN). Kernel weight and grain quality components are presented at 0% moisture.

At the V8 (eighth leaf fully collared) and R1 (beginning of reproduction) growth stages, six plants were sampled (three plants from row 3 and three from row 6). Each corn plant was cut at the base to evaluate the total aboveground biomass. These plants were partitioned into leaves, stalks, and reproductive tissue (reproductive tissue only at R1) and each part was dried, weighed, and ground. The tassel, earshoots, and husks at the R1 growth stage were combined as reproductive tissue. All ground tissue samples were analyzed for N concentration using a combustion technique. The weights of each plant fraction were then utilized to calculate N content within each plant part, as well as total plant N accumulation.

### Example 10 - Improved Plant Nitrogen Consistency

In a series of experiments, KV137 was demonstrated to impart a statistically significant reduction in the variance of whole plant nitrogen compared to an untreated control (“UTC”). The data was taken from 13 separate experiments, each with analogous replicated experimental designs. Experiments were conducted across 10 universities, and included comparing KV137 to a UTC at 4 different nitrogen fertilization rates, shown in Table 16, below. Specifically, the experimental plots were sited in strips with either a full recommended nitrogen application rate (“Full N,” e.g., ~200 lbs N / acre), or with nitrogen rates reduced by either 12.5 lbs N / acre, 25 lbs N / acre, or 50 lbs N / acre. Within nitrogen treatments, UTC and KV137 plots were randomly planted together as replicate blocks. The number of replicates per site and the applicable / associated N rate are presented in Table 16.

At each site, whole plant nitrogen content and whole plant dry weight were sampled at the R1 growth stage, as well as plant population density. These measurements were combined to calculate plant nitrogen weight per acre as follows:

$$\begin{matrix}\text{Plant} \text{N} \left(\text{lb} / \text{acre}\right) = \text{N} \text{content} \left(\%\right) / 100 \text{*} \text{Dry} \text{Weight} \left(\text{lbs} / \text{plant}\right) \text{*} \\ \text{Population} \text{Density} \left(\text{plants} / \text{acre}\right)\end{matrix}$$

TABLE 16

Locations for nitrogen titration trials, with the number of replicate blocks per trial listed by nitrogen rate. Each replicate block contained a control (UTC) plot and a plot with KV137.

| Institution | Location | N (lb / ac) added at Full N | Blocks Full N | Blocks Reduced by 12.5 lbs N / acre | Blocks Reduced by 25 lbs N / acre | Blocks Reduced by 50 lbs N / acre |
|---|---|---|---|---|---|---|
| Arkansas | AR-PTRS | 210 | 6 | 6 | 6 | 6 |
| Clemson | SC-Clemson | 197.5 | 4 | 4 | 4 | 4 |
| Iowa State | IA-Ames | 180 | 3 | 0 | 0 | 0 |
| KSU | KS-Manhattan | 150 | 6 | 6 | 6 | 6 |
| Minnesota | MN-Becker | 225 | 6 | 6 | 6 | 6 |
| Minnesota | MN-Waseca | 150 | 6 | 6 | 6 | 6 |
| NCSU | NC-Plymouth | 180 | 6 | 6 | 6 | 6 |
| Nebraska | NE-Clay | 210 | 6 | 6 | 6 | 6 |
| Purdue | IN-Purdue | 175 | 7 | 7 | 6 | 0 |
| UGA | GA-Sintim | 270 | 4 | 4 | 4 | 4 |
| UI | IL-Champaign | 180 | 7 | 7 | 7 | 7 |
| UI | IL-Nashville | 180 | 6 | 6 | 6 | 6 |
| UI | IL-York Ville | 180 | 9 | 9 | 9 | 9 |

For each location and nitrogen rate, the average difference in variance, standard deviation, and mean N/acre between KV137 and UTC for each nitrogen treatment were calculated (Table 17). A mixed effects model was fit to differences in standard deviation (units of lb / ac), using location as a random effect. Least-squares means estimates of the effect of KV137 on variance at different nitrogen rates are presented in Table 18.

TABLE 17

Changes in variance, standard deviation, and mean value of plant N / acre for KV137 versus the UTC, at varying nitrogen rates.

| Nitrogen Rate | Total # Reps | # Sites | Change in Variance | Change in Std. Dev. | Change in Mean Value |
|---|---|---|---|---|---|
| Full | 76 | 13 | -510 (319.6) | -7.7 (4.8) | 2.8 (4.7) |
| Reduced by 12.5 LBS | 73 | 12 | 106.1 (271.4) | 3.6 (4.2) | 0.9 (5.3) |
| Reduced by 25 LBS | 73 | 12 | -404.7 (211) | -6.2 (2.7) | -7.2 (6.1) |
| Reduced by 50 LBS | 66 | 11 | -246 (221.2) | -3.3 (3.9) | 2.3 (4.4) |

Changes were calculated by subtracting the UTC value from the value for KV137. Thus, a negative value indicates a reduction due to KV137. Standard errors are shown in parentheses.

TABLE 18

Least-Squares (“LS”) Means for a mixed effect model fitted to the difference in standard deviation of whole plant N between KV137 and UTC (calculated as Std. Dev. KV137 - Std. Dev. UTC).

| Nitrogen | LS Means Estimate | Standard Error (SE) | Degrees of freedom (df) | t.ratio | p.value |
|---|---|---|---|---|---|
| Full | -7.75 | 4.13 | 30.9 | -1.86 | 0.07 |
| Reduced by 12.5 LBS | 3.62 | 3.87 | 30.9 | 0.93 | 0.35 |
| Reduced by 25 LBS | -6.19 | 3.87 | 30.9 | -1.60 | 0.11 |
| Reduced by 50 LBS | -3.27 | 3.87 | 30.9 | -0.85 | 0.40 |

Nitrogen variability was significantly reduced in the full N treatment.

For almost all nitrogen rates, the modified strain KV137 reduced the within-site variance and standard deviation of whole plant N at the R1 growth stage, while maintaining elevated average whole plant N compared to the UTC (Table 17). This effect was most pronounced in full N treatments, where the reduction in variance due to KV137 was significant at the 0.10 level.

### Example 11 - Replicated Field Trial Results

A separate set of field trial experiments was conducted using a replicated complete block design with a single nitrogen rate per site. This design included multiple candidate strains alongside KV-137, as well as two non-treated control plots per block and one positive control plot per block (+ 50 lb N /acre). This design was replicated across 25 sites, with each site having 4-6 replicate blocks (specifically, 21 sites with 4 replicates, 4 sites with 6 replicates). Individual plots were reviewed for agronomic issues and included or discarded accordingly. This data was analyzed separately from the nitrogen titration trials above, in view of the difference in experimental design.

Among-plot standard deviation of whole plant nitrogen (lb / acre) at the VT growth stage was calculated for each site and treatment. Standard deviations were analyzed using a mixed effects regression model with site as a random effect and treatment as fixed effect. In the mixed effect model, average standard deviation of KV-137 was slightly larger than that of the control, but this difference was not statistically different from 0 (0.78, p = 0.82, df = 159, t = 0.23).

### Numbered Embodiments of the Disclosure

Notwithstanding the appended claims, the disclosure sets forth the following numbered embodiments:

1. A method for reducing variation in whole plant nitrogen, the method comprising:
providing to a locus a plurality of crop plants and a plurality of remodeled nitrogen fixing microbes that colonize the rhizosphere of said plurality of crop plants and supply the plants with fixed N,
wherein the variation in whole plant nitrogen of the plurality of crop plants colonized by said nitrogen fixing microbes, at a given growth stage and as measured across the locus, is lower than a variation in whole plant nitrogen of a control plurality of crop plants, when the control plurality of crop plants is provided to the locus.

2. The method of embodiment 1, wherein the crop plant is a cereal.

3. The method of any one of the above embodiments, wherein the crop plant is corn, rice, wheat, barley, sorghum, millet, oat, rye, or triticale.

4. The method of any one of the above embodiments, wherein the standard deviation of mean yield for the plurality of crop plants colonized by the remodeled nitrogen fixing microbes is at least about 15 bushels per acre less than the standard deviation of the control plurality of crop plants, said control plurality of crop plants not being colonized by said nitrogen fixing microbes.

5. The method of any one of the above embodiments, wherein the mean yield between the plurality of crop plants colonized by the remodeled nitrogen fixing microbes is within 1-10% of the mean yield of the control plurality of crop plants, said control plurality of crop plants not being colonized by said nitrogen fixing microbes.

6. The method of any one of the above embodiments, wherein the locus comprises agriculturally challenging soil.

7. The method of any one of the above embodiments, wherein the locus comprises soil which is agriculturally challenging as a result of one or more of the following: high sand content; high water content; unfavorable pH; poor drainage; and underperformance, as measured by mean yield of a crop in said underperforming soil compared to mean yield of a crop in a control soil.

8. The method of any one of the above embodiments, wherein the locus comprises an agriculturally challenging soil that comprises at least about 30%, at least about 40%, or at least about 50% sand.

9. The method of any one of the above embodiments, wherein the locus comprises an agriculturally challenging soil that comprises less than about 30% silt.

10. The method of any one of the above embodiments, wherein the locus comprises an agriculturally challenging soil that comprises less than about 20% clay.

11. The method of any one of the above embodiments, wherein the locus comprises an agriculturally challenging soil that comprises a pH of about 5 to about 8.

12. The method of any one of the above embodiments, wherein the locus comprises an agriculturally challenging soil that comprises a pH of about 6.8.

13. The method of any one of the above embodiments, wherein the locus comprises an agriculturally challenging soil that comprises an organic matter content of about 0.40 to about 2.8.

14. The method of any one of the above embodiments, wherein the locus comprises an agriculturally challenging soil that is a sandy loam or loam soil.

15. The method of any one of the above embodiments, wherein the mean yield measured across the locus, in bushels per acre, is higher for the plurality of crop plants colonized by said nitrogen fixing microbes, as compared to a control plurality of crop plants, when the control plurality of crop plants is provided to the locus.

16. The method of any one of the above embodiments, wherein the remodeled nitrogen fixing microbes produce in the aggregate at least about 15 pounds of fixed N per acre over the course of at least about 10 days to about 60 days.

17. The method of any one of the above embodiments, wherein exogenous nitrogen is not applied as a sidedressing to said crop plants.

18. The method of any one of the above embodiments, wherein the remodeled nitrogen fixing microbes each produce fixed N of at least about 2.75 × 10⁻¹² mmol of N per CFU per hour.

19. The method of any one of the above embodiments, wherein the remodeled nitrogen fixing microbes each produce fixed N of at least about 4.03 × 10⁻¹³ mmol of N per CFU per hour.

20. The method of any one of the above embodiments, wherein the remodeled nitrogen fixing microbes colonize the root surface of the plurality of crop plants at a total aggregate CFU per acre concentration of about 5 × 10¹³ for at least about 20 days, 30 days, or 60 days.

21. The method of any one of the above embodiments, wherein the remodeled nitrogen fixing microbes produce 1% or more of the fixed nitrogen in an individual plant of said plurality exposed thereto.

22. The method of any one of the above embodiments, wherein the remodeled nitrogen fixing microbes are capable of fixing atmospheric nitrogen in the presence of exogenous nitrogen.

23. The method of any one of the above embodiments, wherein each member of the plurality of remodeled nitrogen fixing microbes comprises at least one genetic variation introduced into at least one gene, or non-coding polynucleotide, of the nitrogen fixation or assimilation genetic regulatory network.

24. The method of any one of the above embodiments, wherein each member of the plurality of remodeled nitrogen fixing microbes comprises an introduced control sequence operably linked to at least one gene of the nitrogen fixation or assimilation genetic regulatory network.

25. The method of any one of the above embodiments, wherein each member of the plurality of remodeled nitrogen fixing microbes comprises a heterologous promoter operably linked to at least one gene of the nitrogen fixation or assimilation genetic regulatory network.

26. The method of any one of the above embodiments, wherein each member of the plurality of remodeled nitrogen fixing microbes comprises at least one genetic variation introduced into a member selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nijN, nifU, nifS, nifV, nifW, nifZ, nifM, nifF, nifB, nifQ, a gene associated with biosynthesis of a nitrogenase enzyme, and combinations thereof.

27. The method of of any one of the above embodiments, wherein each member of the plurality of remodeled nitrogen fixing microbes comprises at least one genetic variation introduced into at least one gene, or non-coding polynucleotide, of the nitrogen fixation or assimilation genetic regulatory network that results in one or more of: increased expression or activity of NifA or glutaminase; decreased expression or activity of NifL, NtrB, glutamine synthetase, GlnB, GlnK, DraT, AmtB; decreased adenylyl-removing activity of GlnE; or decreased uridylyl-removing activity of GlnD.

28. The method of any one of the above embodiments, wherein each member of the plurality of remodeled nitrogen fixing microbes comprises a mutated nifL gene that comprises a heterologous promoter in said nifL gene.

29. The method of any one of the above embodiments, wherein each member of the plurality of remodeled nitrogen fixing microbes comprises a mutated glnE gene that results in a truncated GlnE protein lacking an adenylyl-removing (AR) domain.

30. The method of any one of the above embodiments, wherein each member of the plurality of remodeled nitrogen fixing microbes comprises a mutated amtB gene that results in the lack of expression of said amtB gene.

31. The method of any one of the above embodiments, wherein each member of the plurality of remodeled nitrogen fixing microbes comprises at least one genetic variation introduced into genes involved in a pathway selected from the group consisting of: exopolysaccharide production, endo-polygalaturonase production, trehalose production, and glutamine conversion.

32. The method of any one of the above embodiments, wherein each member of the plurality of remodeled nitrogen fixing microbes comprises at least one genetic variation introduced into genes selected from the group consisting of: bcsii, bcsiii, yjbE, fhaB, pehA, otsB, treZ, glsA2, and combinations thereof.

33. The method of any one of the above embodiments, wherein the plurality of remodeled nitrogen fixing microbes comprise at least two different species of bacteria.

34. The method of any one of the above embodiments, wherein the plurality of remodeled nitrogen fixing microbes comprise at least two different strains of the same species of bacteria.

35. The method of any one of the above embodiments, wherein the plurality of remodeled nitrogen fixing microbes comprise bacteria selected from: Paenibacillus polymyxa, Paraburkholderia tropica, Herbaspirillum aquaticum, Metakosakonia intestini, Rahnella aquatilis, Klebsiella variicola, Achromobacter spiritinus, Achromobacter marplatensis, Microbacterium murale, Kluyvera intermedia, Kosakonia pseudosacchari, Enterobacter sp., Azospirillum lipoferum, Kosakonia sacchari, and combinations thereof.

36. The method of any one of the above embodiments, wherein the plurality of remodeled nitrogen fixing microbes are epiphytic or rhizospheric.

37. The method of any one of the above embodiments, wherein the plurality of remodeled nitrogen fixing microbes are selected from: bacteria deposited as ATCC PTA-126575, bacteria deposited as ATCC PTA-126576, bacteria deposited as ATCC PTA-126577, bacteria deposited as ATCC PTA-126578, bacteria deposited as ATCC PTA-126579, bacteria deposited as ATCC PTA-126580, bacteria deposited as ATCC PTA-126584, bacteria deposited as ATCC PTA-126586, bacteria deposited as ATCC PTA-126587, bacteria deposited as ATCC PTA-126588, bacteria deposited as NCMA 201701002, bacteria deposited as NCMA 201708004, bacteria deposited as NCMA 201708003, bacteria deposited as NCMA 201708002, bacteria deposited as NCMA 201712001, bacteria deposited as NCMA 201712002, and combinations thereof.

38. The method of any one of the above embodiments, wherein the plurality of remodeled nitrogen fixing microbes comprise bacteria comprising a nucleic acid sequence that shares at least about 90%, 95%, 97%, or 99% sequence identity to a nucleic acid sequence selected from SEQ ID NOs: 177-260, 296-303, and 458-469.

39. The method of any one of the above embodiments, wherein the plurality of remodeled nitrogen fixing microbes comprise bacteria comprising a nucleic acid sequence selected from SEQ ID NOs: 177-260, 296-303, and 458-469.

40. The method of any one of the above embodiments, wherein the remodeled nitrogen fixing microbes from the plurality of remodeled nitrogen fixing microbes are one of transgenic and non-intergeneric.

41. The method of any one of the above embodiments, wherein the variation in the whole plant nitrogen of the plurality of crop plants colonized by the nitrogen fixing microbes, at the given growth stage and as measured across the locus, has a value that is dependent upon an associated nitrogen fertilization rate.

42. The method of embodiment 41, wherein the nitrogen fertilization rate is one of (i) at least about 200 pounds of N per acre or (ii) a standard practice number of pounds of N per acre (e.g., 80, 90, 100, 110, 120, 130, 140, 150, 160, 170, 180, 190, 200, 210, 220, 230, 240, 250, 260, 270, 280, 290, or 300 pounds of N per acre), and results in a reduction in standard deviation of at least about 7 pounds of N per acre.

43. The method of embodiment 41, wherein the nitrogen fertilization rate is one of (i) at least about 150 pounds of N per acre or (ii) about 50 pounds less than a standard practice number of pounds of N per acre, and results in a reduction in standard deviation of at least about 3 pounds of N per acre.

44. The method of embodiment 41, wherein the nitrogen fertilization rate is one of (i) at least about 175 pounds of N per acre or (ii) about 25 pounds less than a standard practice number of pounds of N per acre, and results in a reduction in standard deviation of at least about 6 pounds of N per acre.

45. A plurality of crop plants having reducing variation in whole plant nitrogen, in an agricultural locus, relative to a control set of crop plants, comprising:
a plurality of crop plants in association with a plurality of remodeled nitrogen fixing microbes, whereby the plurality of crop plants receive at least 1% of their in planta fixed N from the remodeled microbes,
wherein the variation in whole plant nitrogen of the plurality of crop plants in association with said nitrogen fixing microbes, at a given growth stage and as measured across the locus, is lower than a variation in whole plant nitrogen of a control plurality of crop plants, when the control plurality of crop plants is provided to the locus.

46. The plurality of crop plants of embodiment 45, wherein the crop plants are cereal plants.

47. The plurality of crop plants of any one of the above embodiments, wherein the crop plants are corn, rice, wheat, barley, sorghum, millet, oat, rye, or triticale plants.

48. The plurality of crop plants of any one of the above embodiments, wherein the standard deviation of mean yield for the plurality of crop plants in association with the remodeled nitrogen fixing microbes is at least about 15 bushels per acre less than the standard deviation of the control plurality of crop plants, said control plurality of crop plants not being in association with the nitrogen fixing microbes.

49. The plurality of crop plants of any one of the above embodiments, wherein the mean yield between the plurality of crop plants in association with the remodeled nitrogen fixing microbes is within 1-10% of the mean yield of the control plurality of crop plants, said control plurality of crop plants not being in association with the nitrogen fixing microbes.

50. The plurality of crop plants of any one of the above embodiments, wherein the locus comprises agriculturally challenging soil.

51. The plurality of crop plants of any one of the above embodiments, wherein the locus comprises agriculturally challenging soil which is agriculturally challenging due to one or more of: high sand content; high water content; unfavorable pH; poor drainage; underperformance relative to a control soil, as measured by mean yield of a crop in said underperforming soil compared to mean yield of a crop in a control soil.

52. The plurality of crop plants of any one of the above embodiments, wherein the locus comprises an agriculturally challenging soil that comprises at least about 30%, at least about 40%, or at least about 50% sand.

53. The plurality of crop plants of any one of the above embodiments, wherein the locus comprises an agriculturally challenging soil that comprises less than about 30% silt.

54. The plurality of crop plants of any one of the above embodiments, wherein the locus comprises an agriculturally challenging soil that comprises less than about 20% clay.

55. The plurality of crop plants of any one of the above embodiments, wherein the locus comprises an agriculturally challenging soil that comprises a pH of about 5 to about 8.

56. The plurality of crop plants of any one of the above embodiments, wherein the locus comprises an agriculturally challenging soil that comprises a pH of about 6.8.

57. The plurality of crop plants of any one of the above embodiments, wherein the locus comprises an agriculturally challenging soil that comprises an organic matter content of about 0.40 to about 2.8.

58. The plurality of crop plants of any one of the above embodiments, wherein the locus comprises an agriculturally challenging soil that is a sandy loam or loam soil.

59. The plurality of crop plants of any one of the above embodiments, wherein the mean yield measured across the locus, in bushels per acre, is higher for the plurality of crop plants in association with the nitrogen fixing microbes, as compared to a control plurality of crop plants, when the control plurality of crop plants is provided to the locus.

60. The plurality of crop plants of any one of the above embodiments, wherein the remodeled nitrogen fixing microbes produce in the aggregate at least about 15 pounds of fixed N per acre over the course of at least about 10 days to about 60 days.

61. The plurality of crop plants of any one of the above embodiments, wherein exogenous nitrogen is not applied as a sidedressing to said crop plants.

62. The plurality of crop plants of any one of the above embodiments, wherein the remodeled nitrogen fixing microbes each produce fixed N of at least about 2.75 × 10⁻¹² mmol of N per CFU per hour.

63. The plurality of crop plants of any one of the above embodiments, wherein the remodeled nitrogen fixing microbes each produce fixed N of at least about 4.03 × 10⁻¹³ mmol of N per CFU per hour.

64. The plurality of crop plants of any one of the above embodiments, wherein the remodeled nitrogen fixing microbes colonize the root surface of the plurality of crop plants at a total aggregate CFU per acre concentration of about 5 × 10¹³ for at least about 20 days, 30 days, or 60 days.

65. The plurality of crop plants of any one of the above embodiments, wherein the remodeled nitrogen fixing microbes are capable of fixing atmospheric nitrogen in the presence of exogenous nitrogen.

66. The plurality of crop plants of any one of the above embodiments, wherein each member of the plurality of remodeled nitrogen fixing microbes comprises at least one genetic variation introduced into at least one gene, or non-coding polynucleotide, of the nitrogen fixation or assimilation genetic regulatory network.

67. The plurality of crop plants of any one of the above embodiments, wherein each member of the plurality of remodeled nitrogen fixing microbes comprises an introduced control sequence operably linked to at least one gene of the nitrogen fixation or assimilation genetic regulatory network.

68. The plurality of crop plants of any one of the above embodiments, wherein each member of the plurality of remodeled nitrogen fixing microbes comprises a heterologous promoter operably linked to at least one gene of the nitrogen fixation or assimilation genetic regulatory network.

69. The plurality of crop plants of any one of the above embodiments, wherein each member of the plurality of remodeled nitrogen fixing microbes comprises at least one genetic variation introduced into a member selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nifN, nifU, nifS, nifV, nifW, nifZ, nifM, nifF, nifB, nifQ, a gene associated with biosynthesis of a nitrogenase enzyme, and combinations thereof.

70. The plurality of crop plants of any one of the above embodiments, wherein each member of the plurality of remodeled nitrogen fixing microbes comprises at least one genetic variation introduced into at least one gene, or non-coding polynucleotide, of the nitrogen fixation or assimilation genetic regulatory network that results in one or more of: increased expression or activity of NifA or glutaminase; decreased expression or activity of NifL, NtrB, glutamine synthetase, GlnB, GlnK, DraT, AmtB; decreased adenylyl-removing activity of GlnE; or decreased uridylyl-removing activity of GlnD.

71. The plurality of crop plants of any one of the above embodiments, wherein each member of the plurality of remodeled nitrogen fixing microbes comprises a mutated nifL gene that comprises a heterologous promoter in said nifL gene.

72. The plurality of crop plants of any one of the above embodiments, wherein each member of the plurality of remodeled nitrogen fixing microbes comprises a mutated glnE gene that results in a truncated GlnE protein lacking an adenylyl-removing (AR) domain.

73. The plurality of crop plants of any one of the above embodiments, wherein each member of the plurality of remodeled nitrogen fixing microbes comprises a mutated amtB gene that results in the lack of expression of said amtB gene.

74. The plurality of crop plants of any one of the above embodiments, wherein each member of the plurality of remodeled nitrogen fixing microbes comprises at least one genetic variation introduced into genes involved in a pathway selected from the group consisting of: exopolysaccharide production, endo-polygalaturonase production, trehalose production, and glutamine conversion.

75. The plurality of crop plants of any one of the above embodiments, wherein each member of the plurality of remodeled nitrogen fixing microbes comprises at least one genetic variation introduced into genes selected from the group consisting of: bcsii, bcsiii, yjbE, fhaB, pehA, otsB, treZ, glsA2, and combinations thereof.

76. The plurality of crop plants of any one of the above embodiments, wherein the plurality of remodeled nitrogen fixing microbes comprise at least two different species of bacteria.

77. The plurality of crop plants of any one of the above embodiments, wherein the plurality of remodeled nitrogen fixing microbes comprise at least two different strains of the same species of bacteria.

78. The plurality of crop plants of any one of the above embodiments, wherein the plurality of remodeled nitrogen fixing microbes comprise bacteria selected from: Paenibacillus polymyxa, Paraburkholderia tropica, Herbaspirillum aquaticum, Metakosakonia intestini, Rahnella aquatilis, Klebsiella variicola, Achromobacter spiritinus, Achromobacter marplatensis, Microbacterium murale, Kluyvera intermedia, Kosakonia pseudosacchari, Enterobacter sp., Azospirillum lipoferum, Kosakonia sacchari, and combinations thereof.

79. The plurality of crop plants of any one of the above embodiments, wherein the plurality of remodeled nitrogen fixing microbes are epiphytic or rhizospheric.

80. The plurality of crop plants of any one of the above embodiments, wherein the plurality of remodeled nitrogen fixing microbes are selected from: bacteria deposited as ATCC PTA-126575, bacteria deposited as ATCC PTA-126576, bacteria deposited as ATCC PTA-126577, bacteria deposited as ATCC PTA-126578, bacteria deposited as ATCC PTA-126579, bacteria deposited as ATCC PTA-126580, bacteria deposited as ATCC PTA-126584, bacteria deposited as ATCC PTA-126586, bacteria deposited as ATCC PTA-126587, bacteria deposited as ATCC PTA-126588, bacteria deposited as NCMA 201701002, bacteria deposited as NCMA 201708004, bacteria deposited as NCMA 201708003, bacteria deposited as NCMA 201708002, bacteria deposited as NCMA 201712001, bacteria deposited as NCMA 201712002, and combinations thereof.

81. The plurality of crop plants of any one of the above embodiments, wherein the plurality of remodeled nitrogen fixing microbes comprise bacteria comprising a nucleic acid sequence that shares at least about 90%, 95%, 97%, or 99% sequence identity to a nucleic acid sequence selected from SEQ ID NOs: 177-260, 296-303, and 458-469.

82. The plurality of crop plants of any one of the above embodiments, wherein the plurality of remodeled nitrogen fixing microbes comprise bacteria comprising a nucleic acid sequence selected from SEQ ID NOs: 177-260, 296-303, and 458-469.

83. A processor-implemented method for determining a quantity of a crop plant to sell based on whole plant nitrogen variability data for a bacteria-colonized plant, the method comprising:
retrieving, via a processor and from a database operably coupled to the processor, whole plant nitrogen variability data for a bacteria-colonized plant, the whole plant nitrogen variability data including a whole plant nitrogen variabilitythat is lower than a whole plant nitrogen variability of a plant that has not been bacterially colonized;
retrieving, via a processor and from a database operably coupled to the processor, a price associated with a current and future sale of a quantity of the crop plant;
calculating, via the processor, a physical delivery quantity of the bacteria-colonized plant based on the whole plant nitrogen variability data for the bacteria-colonized plant and the current and future sale price;
identifying a market-based instrument based on the calculated physical delivery quantity of the bacteria-colonized plant;
sending, via the processor, a signal representing an instruction to transact the identified market-based instrument; and
receiving, at the processor and in response to sending the instruction to transact the identified market-based instrument, a signal representing a confirmation of a transaction of the identified market-based instrument.

84. The processor-implemented method of embodiment 83, wherein the calculating the physical delivery quantity is performed prior to a growing season associated with the bacteria-colonized plant.

85. The processor-implemented method of any one of the above embodiments, wherein the transaction of the identified market-based instrument is performed prior to a growing season associated with the bacteria-colonized plant.

86. The processor-implemented method of any one of the above embodiments, wherein the market-based instrument is a forward contract.

87. The processor-implemented method of any one of the above embodiments, wherein the market-based instrument is a futures contract.

88. The processor-implemented method of any one of the above embodiments, wherein the market-based instrument is an options contract.

89. The processor-implemented method of any one of the above embodiments, wherein the market-based instrument is a commodity swap contract.

90. The processor-implemented method of any one of the above embodiments, wherein the instruction to transact the identified market-based instrument comprises a trading symbol.

91. The processor-implemented method of any one of the above embodiments, wherein the transaction of the identified market-based instrument occurs within a secondary market.

92. The processor-implemented method of any one of the above embodiments, further comprising: producing the physical delivery quantity of the bacteria-colonized plant.

93. The processor-implemented method of embodiment 92, wherein producing the bacteria-colonized plant comprises:
a. providing to a locus a plurality of non-intergeneric remodeled bacteria that each produce fixed N of at least about 5.49 × 10⁻¹³ mmol of N per CFU per hour; and
b. providing to the locus the pre-colonization plant.

94. The processor-implemented method of any one of the above embodiments, wherein the bacteria-colonized plant is a corn plant.

95. The processor-implemented method of any one of the above embodiments, wherein the bacteria-colonized plant is produced using an engineered N fixing microbe.

96. The processor-implemented method of any one of the above embodiments, wherein the bacteria-colonized plant is produced using biological nitrogen fixation.

97. The processor-implemented method of any one of the above embodiments, wherein the bacteria-colonized plant is produced using a microorganism capable of fixing atmospheric nitrogen for associated crops.

98. The processor-implemented method of any one of the above embodiments, wherein the signal representing the confirmation of the transaction of the identified market-based instrument is received at the processor via an application programming interface (API).

99. The processor-implemented method of any one of the above embodiments, wherein the database includes corn yield data.

100. The processor-implemented method of any one of the above embodiments, wherein the standard deviation associated with the yield value is measured in bushels per acre.

101. The processor-implemented method of any one of the above embodiments, wherein the standard deviation associated with the yield value is less than 19 bushels per acre.

102. The processor-implemented method of any one of the above embodiments, wherein the yield value for the bacteria-colonized plant is within 1-10% of the yield value of the plant that has not been bacterially colonized.

103. The processor-implemented method of any one of the above embodiments, wherein the physical delivery quantity of the bacteria-colonized plant is a predicted physical delivery quantity of the bacteria-colonized plant.

104. The processor-implemented method of embodiment 103, wherein the predicted physical delivery quantity of the bacteria-colonized plant includes a predicted quantity of bacteria-colonized plants grown on land that has historically produced a lower yield of the plant that has not been bacterially colonized.

105. A processor-implemented method for pricing and transacting an insurance product, the method comprising:
receiving, via a processor, information about a proposed insurance product; and
calculating, via the processor, a price for the proposed insurance product based on nitrogen variability data for a bacteria-colonized plant, the nitrogen variability data including a nitrogen variability that is lower than a nitrogen variability of a plant that has not been bacterially colonized.

106. The processor-implemented method of embodiment 105, further comprising:
sending, via the processor and from a compute device of a seller, a signal representing an offer to sell insurance, the offer to sell insurance including the calculated price for the proposed insurance product; and
receiving, at the processor and in response to sending the price for the proposed insurance product, a signal representing an acceptance of the offer to sell insurance.

107. The processor-implemented method of any one of the above embodiments, wherein the calculating the price for the proposed insurance product is performed prior to a growing season associated with the bacteria-colonized plant.

108. The processor-implemented method of any one of the above embodiments, wherein the sending the signal representing the offer to sell insurance is performed prior to a growing season associated with the bacteria-colonized plant.

109. The processor-implemented method of any one of the above embodiments, wherein the yield value is based on a production of the bacteria-colonized plant by a process comprising:
a. providing to a locus a plurality of non-intergeneric remodeled bacteria that each produce fixed N of at least about 5.49 × 10⁻¹³ mmol of N per CFU per hour; and
b. providing to the locus a pre-colonization plant.

110. The processor-implemented method of any one of the above embodiments, further comprising producing the bacteria-colonized plant, using a pre-colonization plant, by:
a. providing to a locus a plurality of non-intergeneric remodeled bacteria that each produce fixed N of at least about 5.49 × 10⁻¹³ mmol of N per CFU per hour; and
b. providing to the locus the pre-colonization plant.

111. The processor-implemented method of any one of the above embodiments, wherein the bacteria-colonized plant is a corn plant.

112. The processor-implemented method of any one of the above embodiments, wherein the yield value is based on producing the bacteria-colonized plant by a process comprising using an engineered N fixing microbe.

113. The processor-implemented method of any one of the above embodiments, wherein the yield value is based on producing the bacteria-colonized plant by a process comprising using biological nitrogen fixation.

114. The processor-implemented method of any one of the above embodiments, wherein the yield value is based on producing the bacteria-colonized plant by a process comprising using a microorganism capable of fixing atmospheric nitrogen for associated crops.

115. The processor-implemented method of any one of the above embodiments, wherein the signal representing the offer to sell insurance is sent via an application programming interface (API).

116. The processor-implemented method of any one of the above embodiments, wherein the signal representing acceptance of the offer to sell insurance is received via an API.

117. The processor-implemented method of any one of the above embodiments, wherein the signal representing the offer to sell insurance further comprises the yield value for the bacteria-colonized plant.

118. A method of increasing the value of a commodity, the method comprising:

decreasing nitrogen variability of the commodity by growing the commodity in the presence of a nutrient-providing microorganism.

119. The method of embodiment 118, further comprising:

determining a plurality of different prices for sale of the commodity, for each of multiple markets in which the commodity can be sold.

120. The method of any one of the above embodiments, wherein decreasing the variability in yield of the commodity allows a seller of the commodity to increase sales of the commodity into markets with higher pricing for the commodity, or allows the seller of the commodity to decrease sales of the commodity into markets with lower pricing for the commodity.

121. The method of any one of the above embodiments, wherein the markets with higher pricing for the commodity comprise markets that occur prior to a production season for the commodity.

122. The method of any one of the above embodiments, wherein the markets with lower pricing for the commodity comprise markets that occur after a production season for the commodity.

123. The method of any one of the above embodiments, wherein the commodity is a crop plant.

124. The method of any one of the above embodiments, wherein growing the crop plant in the presence of the nutrient-providing microorganism improves the availability of the provided nutrient to the crop plant.

125. The method of any one of the above embodiments, wherein the crop plant is corn.

126. The method of any one of the above embodiments, wherein the one or more nutrients includes nitrogen, and the microorganism is a nitrogen-fixing bacterium.

127. The method of any one of the above embodiments, wherein the variability in yield of the commodity comprises variability in yield of the commodity across a farmer’s field.

128. The method of any one of the above embodiments, wherein the variability in yield of the commodity is substantially due to variability in response to weather conditions.

129. A method of decreasing insurance costs for a commodity, the method comprising:

decreasing nitrogen variability of the commodity by growing the commodity in the presence of a nutrient-providing microorganism.

130. The method of any one of the above embodiments, wherein the commodity is a crop plant.

131. The method of any one of the above embodiments, wherein growing the crop plant in the presence of the nutrient-providing microorganism improves the availability of the provided nutrient to the crop plant.

132. The method of any one of the above embodiments, wherein the crop plant is corn.

133. The method of any one of the above embodiments, wherein the one or more nutrients includes nitrogen, and the microorganism is a nitrogen-fixing bacterium.

134. The method of any one of the above embodiments, wherein the variability in yield of the commodity includes variability in yield of the commodity across a farmer’s field.

135. The method of any one of the above embodiments, wherein the variability in yield of the commodity is substantially due to variability in response to weather conditions.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following Claims define the scope of the disclosure and that methods and structures within the scope of these claims and their equivalents be covered thereby.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world. Further, U.S. Pat. No. 9,975,817, issued on May 22, 2018, and entitled: Methods and Compositions for Improving Plant Traits, is hereby incorporated by reference. Further, PCT/US2018/013671, filed Jan. 12, 2018, published as WO2018/132774A1 on Jul. 19, 2018, and entitled: Methods and Compositions for Improving Plant Traits, is hereby incorporated by reference. Further, PCT/US2019/041429, filed Jul. 11, 2019, and entitled: Temporally and Spatially Targeted Dynamic Nitrogen Delivery by Remodeled Microbes, is hereby incorporated by reference. Further, PCT/US2020/016471, filed Feb. 4, 2020 and titled improved Consistency of Crop Yield Through Biological Nitrogen Fixation, is hereby incorporated by reference.

What is claimed is:

1. A method for reducing variation in whole plant nitrogen, the method comprising:
providing to a locus a plurality of crop plants and a plurality of nitrogen fixing microbes that colonize the rhizosphere of said plurality of crop plants and supply the plants with fixed N,
wherein the variation in whole plant nitrogen of the plurality of crop plants colonized by said nitrogen fixing microbes, at a given growth stage and as measured across the locus, is lower than a variation in whole plant nitrogen of a control plurality of crop plants, when the control plurality of crop plants is provided to the locus.

2. The method of claim 1, wherein the plurality of nitrogen fixing microbes includes at least one of a wild type microbe, an engineered microbe, a transgenic microbe, an intragenic microbe, a remodeled microbe, and a non-intergeneric remodeled microbe.

3. The method of claim 1, wherein the given growth stage is a vegetative growth stage between V1 and V9, inclusive.

4. (canceled)

5. The method of claim 1, wherein the given growth stage is a reproductive growth stage between R1 and R6, inclusive.

6. (canceled)

7. The method of claim 1, wherein the nitrogen fixing microbes are provided via in-furrow treatment or as an on seed treatment.

8. The method of claim 1, wherein the variation in whole plant nitrogen of the plurality of crop plants colonized by the nitrogen fixing microbes is at least about 15% lower than the variation in whole plant nitrogen of the control plurality of crop plants.

9. The method of claim 1, wherein the crop plant is a cereal.

10. The method of claim 1, wherein the locus comprises agriculturally challenging soil.

11. The method of claim 1, wherein the nitrogen fixing microbes:
produce in the aggregate at least about 15 pounds of fixed N per acre over the course of at least about 10 days to about 60 days; or
each produce fixed N of at least about 2.75 × 10⁻¹² mmol of N per CFU per hour.

12. (canceled)

13. The method of claim 1, wherein the nitrogen fixing microbes are capable of fixing atmospheric nitrogen in the presence of exogenous nitrogen.

14. The method of claim 1, wherein each member of the plurality of nitrogen fixing microbes comprises at least one genetic variation introduced into at least one gene, or non-coding polynucleotide, of the nitrogen fixation or assimilation genetic regulatory network.

15. (canceled)

16. (canceled)

17. The method of claim 1, wherein the variation in the whole plant nitrogen of the plurality of crop plants colonized by the nitrogen fixing microbes, at the given growth stage and as measured across the locus, has a value that is dependent upon an associated nitrogen fertilization rate, and wherein the nitrogen fertilization rate is one of:
a) at least about 200 pounds of N per acre or a standard practice number of pounds of N per acre, and results in a reduction in standard deviation of at least about 7 pounds of N per acre;
b) at least about 150 pounds of N per acre or about 50 pounds less than a standard practice number of pounds of N per acre, and results in a reduction in standard deviation of at least about 3 pounds of N per acre; or
c) at least about 175 pounds of N per acre or about 25 pounds less than a standard practice number of pounds of N per acre, and results in a reduction in standard deviation of at least about 6 pounds of N per acre.

18-20. (canceled)

21. A plurality of crop plants having reduced variation in whole plant nitrogen, in an agricultural locus, relative to a control set of crop plants, comprising:
a plurality of crop plants at a given growth stage, in association with a plurality of nitrogen fixing microbes, whereby the plurality of crop plants receive at least 1% of their in planta fixed N from the microbes,
wherein the variation in whole plant nitrogen measured across the locus is lower for the plurality of crop plants in association with said nitrogen fixing microbes, as compared to a control plurality of crop plants, when the control plurality of crop plants at the given growth stage is provided to the locus.

22. The plurality of crop plants of claim 21, wherein the plurality of nitrogen fixing microbes includes at least one of a wild type microbe, an engineered microbe, a transgenic microbe, an intragenic microbe, a remodeled microbe, and a non-intergeneric remodeled microbe.

23. The plurality of crop plants of claim 21, wherein the given growth stage is a vegetative growth stage between V1 and V9, inclusive.

24. (canceled)

25. The plurality of crop plants of claim 21, wherein the given growth stage is a reproductive growth stage between R1 and R6, inclusive.

26-27. (canceled)

28. The plurality of crop plants of claim 21, wherein the variation in whole plant nitrogen of the plurality of crop plants in association with the nitrogen fixing microbes is at least about 15% lower than that of the control plurality of crop plants.

29. The plurality of crop plants of claim 21, wherein the crop plants are cereal plants.

30. The plurality of crop plants of claim 21, wherein the locus comprises agriculturally challenging soil.

31. The plurality of crop plants of claim 21, wherein the nitrogen fixing microbes:
produce in the aggregate at least about 15 pounds of fixed N per acre over the course of at least about 10 days to about 60 days; or
each produce fixed N of at least about 2.75 × 10⁻¹² mmol of N per CFU per hour.

32. (canceled)

33. The plurality of crop plants of claim 21, wherein the nitrogen fixing microbes are capable of fixing atmospheric nitrogen in the presence of exogenous nitrogen.

34. The plurality of crop plants of claim 21, wherein each member of the plurality of nitrogen fixing microbes comprises at least one genetic variation introduced into at least one gene, or non-coding polynucleotide, of the nitrogen fixation or assimilation genetic regulatory network.

35-36. (canceled)

37. A processor-implemented method for determining a quantity of a crop plant to sell based on whole plant nitrogen variability data for a bacteria-colonized plant, the method comprising:
retrieving, via a processor and from a database operably coupled to the processor, whole plant nitrogen variability data for a bacteria-colonized plant, the whole plant nitrogen variability data including a whole plant nitrogen variability that is lower than a whole plant nitrogen variability of a plant that has not been bacterially colonized;
retrieving, via a processor and from a database operably coupled to the processor, a price associated with a current and future sale of a quantity of the crop plant;
calculating, via the processor, a physical delivery quantity of the bacteria-colonized plant based on the whole plant nitrogen variability data for the bacteria-colonized plant and the current and future sale price;
identifying a market-based instrument based on the calculated physical delivery quantity of the bacteria-colonized plant;
sending, via the processor, a signal representing an instruction to transact the identified market-based instrument; and
receiving, at the processor and in response to sending the instruction to transact the identified market-based instrument, a signal representing a confirmation of a transaction of the identified market-based instrument.

38. The processor-implemented method of claim 37, further comprising: producing the physical delivery quantity of the bacteria-colonized plant.

39. The processor-implemented method of claim 38, wherein producing the physical delivery quantity of the bacteria-colonized plant comprises:
a. providing to a locus a plurality of non-intergeneric remodeled bacteria that each produce fixed N of at least about 5.49 × 10⁻¹³ mmol of N per CFU per hour; and
b. providing to the locus the pre-colonization plant.

40. A processor-implemented method for pricing and transacting an insurance product, the method comprising:
receiving, via a processor, information about a proposed insurance product; and
calculating, via the processor, a price for the proposed insurance product based on nitrogen variability data for a bacteria-colonized plant, the nitrogen variability data including a nitrogen variability that is lower than a nitrogen variability of a plant that has not been bacterially colonized.

41. The processor-implemented method of claim 40, further comprising:
sending, via the processor and from a compute device of a seller, a signal representing an offer to sell insurance, the offer to sell insurance including the calculated price for the proposed insurance product; and
receiving, at the processor and in response to sending the price for the proposed insurance product, a signal representing an acceptance of the offer to sell insurance.

42. The processor-implemented method of claim 40, wherein the nitrogen variability data is based on a production of the bacteria-colonized plant by a process comprising:
a. providing to a locus a plurality of non-intergeneric remodeled bacteria that each produce fixed N of at least about 5.49 × 10⁻¹³ mmol of N per CFU per hour; and
b. providing to the locus a pre-colonization plant.

43. A method of increasing the value of a commodity, the method comprising:
decreasing nitrogen variability of the commodity by growing the commodity in the presence of a nutrient-providing microorganism.

44. The method of claim 43, wherein the commodity is a crop plant, and growing the crop plant in the presence of the nutrient-providing microorganism improves the availability of the provided nutrient to the crop plant.

45. A method of decreasing insurance costs for a commodity, the method comprising:
decreasing nitrogen variability of the commodity by growing the commodity in the presence of a nutrient-providing microorganism.

46. The method of claim 45, wherein the commodity is a crop plant, and growing the crop plant in the presence of the nutrient-providing microorganism improves the availability of the provided nutrient to the crop plant.
